(19) United States
(12) Patent Application Publication
(10) Pub. No.: US 2005/0182060 A1
(43) Pub. Date: Aug. 18, 2005

(54) 2-Substituted and 4-substituted aryl nitrone compounds

(76) Inventors: Michael G. Kelly, Thousand Oaks, CA (US); John Kincaid, Foster City, CA (US); Satyanarayana Janagani, Santa Clara, CA (US)

Correspondence Address: JONES DAY, 222 EAST 41ST ST, NEW YORK, NY, 10017, US

(21) Appl. No.: 11/056,443

(22) Filed: Feb. 10, 2005

Related U.S. Application Data
(60) Provisional application No. 60/544,764, filed on Feb. 13, 2004.
(60) Provisional application No. 60/544,765, filed on Feb. 13, 2004.
(60) Provisional application No. 60/544,766, filed on Feb. 13, 2004.
(60) Provisional application No. 60/545,616, filed on Feb. 17, 2004.
(60) Provisional application No. 60/562,509, filed on Apr. 14, 2004.

(51) Int. Cl. A61K 31/675; C07D 471/02; A61K 31/655; A61K 31/503; A61K 31/498; A61K 31/405; C07D 487/02

(52) U.S. Cl. 514/248; 514/249; 514/300; 514/419; 544/236; 544/330; 546/113; 548/503; 514/151; 514/81

(57) ABSTRACT

The present invention provides aryl nitrones, compositions comprising the same and methods of their use for the treatment or prevention of oxidative, ischemic, ischemia/reperfusion-related and chemokine mediated conditions.

This application claims the benefit of priority under 35 U.S.C. § 119(e) of U.S. provisional application Nos. 60/544,764, 60/544,765, 60/544,766, 60/545,616 and 60/562,509, the contents of which are hereby incorporated by reference in their entireties.

### 1. FIELD OF THE INVENTION

The present invention provides orally active nitrone compounds useful for the treatment and the prevention of free radical mediated conditions, ischemic conditions and ischemia/reperfusion related conditions, and chemokine mediated conditions.

### 2. BACKGROUND OF THE INVENTION

Numerous conditions that afflict human subjects are mediated by oxidative and/or free radical mechanisms. Such conditions include, but are not limited to, neurological, neurodegenerative, inflammatory, autoimmune and pain conditions. Prominent examples include stroke, arteriosclerosis and other cardiovascular diseases, myocardial infarction and dysfunction, multiple sclerosis, head trauma and traumatic brain injury, nerve injury and neuropathies, pain (acute and chronic or neuropathic), arthritis and other autoimmune disorders, and asthma and allergic reactions. There is an ongoing need for the development of compounds, pharmaceutical compositions and methods of treatment for these conditions.

Nitrones constitute a class of compounds that are believed to have antioxidant properties due to their ability to form stable adducts (i.e., spin traps) with free radicals. Since oxidative species and/or free radicals can cause oxidative damage to cellular constituents (e.g., proteins and lipids), which can lead to pathological consequences, it has been reported that the antioxidant properties of nitrones at least partly underlie their therapeutic potential. Therefore, diseases which have been reported to be susceptible to antioxidant therapy or which involve the generation of free radicals may be susceptible to nitrone treatment based on the antioxidant activity of nitrones.

Aromatic nitrone compounds such as C-(phenyl)-N-(tert-butyl)nitrone (PBN) and derivatives thereof have been reported as possible therapeutics for the treatment of a wide variety of disease conditions arising from or characterized by oxidative damage or oxidative stress. Nitrone compounds exhibiting improved antioxidant activity compared to PBN can have better therapeutic potential than PBN. Aromatic nitrone breakdown, metabolism or degradation products such as N-alkyl hydroxylamines, N-alkyl hydronitroxides or nitric oxide may also contribute to the antioxidant properties of the aromatic nitrones, and contribute to their interruption of the inflammatory signaling pathways. One nitrone, C-(2,4-disulfo-phenyl)-N-(tert-butyl)nitrone, disodium salt (Cerovive®) is currently being evaluated in phase III clinical trials for the treatment of acute ischemic stroke. See U.S. Pat. No. 5,475,032.

A need exists for new classes of aromatic nitrone derivatives that have improved properties such as low toxicity, increased solubility, improved cellular and blood-brain-barrier permeability, and improved oral bioavailability.

### 3. SUMMARY OF THE INVENTION

The present invention provides 2-substituted and 4-substituted aryl nitrones that display surprisingly high oral bioavailability and surprisingly low toxicity. The aryl nitrones of the invention, as described in the examples below, can show high oral bioavailability and high in vivo half life. With such outstanding bioavailability, the compounds of the present invention are useful as oral therapeutics for the treatment and prevention of diseases, such as oxidative, ischemic, ischemia/reperfusion-related and chemokine mediated diseases, in a subject.

In a first aspect, the present invention provides 2-substituted aryl nitrones that, in certain embodiments, show high oral bioavailability. The compounds comprise an aryl group or a heteroaryl group bonded to the carbon atom of a nitrone group. The nitrone carbon can be further bonded to hydrogen, lower alkyl or alkyl, and the nitrone nitrogen can be bonded to lower alkyl, alkyl, aryl, arylalkyl, cycloalkyl, heteroaryl, heteroarylalkyl or cycloheteroalkyl. The aryl group or heteroaryl group can be any aryl or heteroaryl known to those of skill in the art. Preferred aryl or heteroaryl groups comprise a six-membered ring bonded to the nitrone. Significantly, in these aryl nitrones of the invention, the aryl or heteroaryl group is substituted with one or more substituents selected from the group consisting of sulfone, carboxyl, aminocarbonyl and tetrazole, at least one of these susbstituents is at an ortho or 2-position of the aryl ring relative to the nitrone group. In preferred embodiments, the compound is not one of compounds 201-204, described below.

In certain embodiments, the present invention provides 2-substituted aryl nitrones according to formula I: 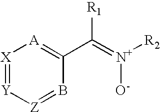
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
at least one of A and B is C—R³, and the other is selected from C—R³ and N;
at least one R³ is SO₂R⁵, CO₂R⁵, CONR⁵R⁶ or tetrazole, and any other R³ is independently selected from R⁴, H, lower alkyl, alkenyl, alkyl, halogen, aryl, SO₂R⁵, SO₂NR⁵R⁶, CO₂H, CONR⁵R⁶ and tetrazole;
X, Y and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, and where feasible may join together to form a saturated or unsaturated cycloheteroalkyl ring containing 4 to 8 atoms, optionally having one or more heteroatoms selected from the list NR¹, O or S.

In further embodiments, the present invention provides compounds according to formula (I), wherein the compounds do not encompass any of compounds 201 through 204, below.

In a second aspect, the present invention provides aryl nitrones that, in certain embodiments, show high oral bioavailability. The compounds comprise an aryl group or a heteroaryl group bonded to the carbon atom of a nitrone group. The nitrone carbon can be further bonded to hydrogen, lower alkyl or alkyl, and the nitrone nitrogen can be bonded to lower alkyl, alkyl, aryl, arylalkyl, cycloalkyl, heteroaryl, heteroarylalkyl or cycloheteroalkyl. The aryl group or heteroaryl group can be any aryl or heteroaryl known to those of skill in the art. Preferred aryl or heteroaryl groups comprise a six-membered ring bonded to the nitrone. Significantly, in these aryl nitrones of the invention, the aryl or heteroaryl group is substituted with one or more sulfonamide, and at least one of these sulfonamides is at an ortho or 2-position of the aryl ring relative to the nitrone group.

In certain embodiments, the present invention provides 2-sulfonamidyl aryl nitrones according to formula II: 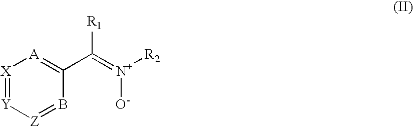
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
at least one of A and B is C—R³, and the other is selected from C—R³ and N;
at least one R³ is SO₂NR⁵R⁶, and any other R³is independently selected from R⁴, H, lower alkyl, alkenyl, alkyl, halogen, aryl, SO₂NR⁵R⁶, SO₂R⁵, CO₂H, CONR⁵R⁶ and tetrazole;
X, Y and Z are each independently selected from C—R⁴ and N;
each R⁴is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, and where feasible may join together to form a saturated or unsaturated cycloheteroalkyl ring containing 4 to 8 atoms, optionally having one or more heteroatoms selected from the list NR¹, O or S.

In a third aspect, the present invention provides 4-substituted aryl nitrones that, in certain embodiments, show high oral bioavailability. The compounds comprise an aryl group or a heteroaryl group bonded to the carbon atom of a nitrone group. The nitrone carbon can be further bonded to hydrogen, lower alkyl or alkyl, and the nitrone nitrogen can be bonded to lower alkyl, alkyl, aryl, arylalkyl, cycloalkyl, heteroaryl, heteroarylalkyl or cycloheteroalkyl. The aryl group or heteroaryl group can be any aryl or heteroaryl known to those of skill in the art. Preferred aryl or heteroaryl groups comprise a six-membered ring bonded to the nitrone. Significantly, in these aryl nitrones of the invention, the aryl or heteroaryl group is substituted with one or more substituents selected from the group consisting of sulfonamide, sulfone, carboxyl, aminocarbonyl and tetrazole, and at least one of these substituents is at para or 4-position of the aryl ring relative to the nitrone group. In preferred embodiments, the compound is not one of compounds 401-426, described below. Preferred compounds include 4-sulfonamide substituted compounds and 4-sulfonyl compounds.

In another aspect, the present invention provides 4-substituted aryl nitrones according to formula III: 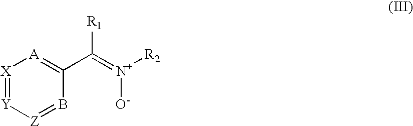
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
Y is C—R⁹, and R⁹ is selected from SO₂NR⁵R⁶, SO₂R⁵, CO₂R⁵, CONR⁵R⁶ and tetrazole;
A, B, X and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, and where feasible may join together to form a saturated or unsaturated cycloheteroalkyl ring containing 4 to 8 atoms, optionally having one or more heteroatoms selected from the list NR¹, O or S.

In another aspect, the present invention provides pharmaceutical compositions comprising an aryl nitrone of the invention. The pharmaceutical compositions of the invention comprise an amount of the aryl nitrone effective to treat or prevent an oxidative, ischemic, ischemia/reperfusion-related or chemokine mediated condition in a subject. The compositions may be administered by a variety of routes, including, by example, orally and parenterally. In advantageous embodiments, the compounds are formulated for oral administration.

In a further aspect, the present invention provides unit dosage forms of an aryl nitrone of the invention for treating or preventing an oxidative, ischemic, ischemia/reperfusion-related or chemokine mediated condition in a subject. In certain embodiments the unit dosage forms comprise a pharmaceutical composition of an aryl nitrone in an amount effective to treat or prevent oxidative, ischemic, ischemia/reperfusion-related or chemokine mediated condition in a subject.

In a method of treatment or prophylaxis aspect, this invention provides a method of treating or prophylaxing a mammal susceptible to or afflicted with an oxidative, ischemic or ischemia/reperfusion-related condition. Exemplary conditions include, but are not limited to, neurological, cardiovascular and organ transplant-related conditions. The method comprises administering an effective amount of one or more of the aryl nitrones or pharmaceutical compositions described above. The compounds can be administered according to any technique known to those of skill in the art. In advantageous embodiments, the compounds are administered orally.

In a further method of treatment prohpylaxis aspect, the present invention provides a method of treating or prophylaxing a mammal susceptible to or afflicted with a condition modulated by a chemokine function or activity. Such conditions include, but are not limited to, neurodegenerative disease, peripheral neuropathies, infections, sequelae of infections and autoimmune diseases. The method comprises administering an effective amount of one or more of the aryl nitrones or pharmaceutical compositions described above.

In additional aspects, this invention provides methods for synthesizing the aryl nitrones of the invention.

### 4. BRIEF DESCRIPTION OF THE FIGURES

FIG. 1 provides reversal of mechanical hyperalgesia by Compound 62 in rat;

FIG. 2 provides reversal of allodynia by Compound 62 in the rat;

FIG. 3 provides anti-allodynic effects of Compound 62 in the rat;

FIG. 4 provides total infarct volume at 48 hrs for animals treated with compounds 62, 20 and 63;

FIG. 5 provides total infarct volume at 48 hrs for animals treated with Compound 62; and

FIG. 6 provides total infarct volume at 48 hrs for animals treated with Compound 63.

### 5. DETAILED DESCRIPTION OF THE INVENTION

The present invention is based, in part, on the discovery that the aryl nitrones of the invention that, in certain embodiments, display surprising oral bioavailability and surprisingly low toxicity. Accordingly, the present invention provides the aryl nitrones, compositions comprising the aryl nitrones and methods of their use for treating or preventing oxidative, ischemic, ischemia/reperfusion-related or chemokine mediated disorders.

5.1 Definitions

When describing the aryl nitrones, pharmaceutical compositions and methods of this invention, the following terms have the following meanings unless otherwise specified.

“Acyl” refers to the group —C(O)R where R is hydrogen, alkyl, aryl or cycloalkyl.

“Acylamino” refers to the group —NRC(O)R where each R is independently hydrogen, alkyl, aryl or cycloalkyl.

“Acyloxy” refers to the group —OC(O)R where R is hydrogen, alkyl, aryl or cycloalkyl.

“Alkenyl” refers to a monovalent branched or unbranched unsaturated hydrocarbon group preferably having from 2 to 10 carbon atoms and more preferably 2 to 8 carbon atoms and having at least 1 and preferably from 1-2 sites of carbon-carbon double bond unsaturation. Preferred alkenyl groups include ethenyl (—CH═CH₂), n-propenyl (—CH₂CH═CH₂), isopropenyl (—C(CH₃)═CH₂), and the like.

“Substituted alkenyl” refers to an alkenyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR where R is alkyl. Preferred alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” refers to an alkoxy group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxycarbonyl” refers to the group —C(O)OR where R is alkyl or cycloalkyl.

“Alkoxycarbonylamino” refers to the group —NRC(O)OR′ where R is hydrogen, alkyl, aryl or cycloalkyl, and R′ is alkyl or cycloalkyl.

“Alkyl” refers to a monovalent branched or unbranched saturated hydrocarbon group preferably having from 1 to about 11 carbon atoms, more preferably from 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to an alkyl group having from 1 to 11 carbon atoms.

“Substituted alkyl” refers to an alkyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkylene” refers to a divalent branched or unbranched saturated hydrocarbon group preferably having from 1 to 10 carbon atoms and more preferably from 1 to 6 carbon atoms. This term is exemplified by groups such as methylene (—CH₂—), ethylene (—CH₂CH₂—), the propylene isomers (e.g., CH₂CH₂CH₂— and —CH(CH₃)CH₂—) and the like.

“Substituted alkylene” refers to an alkylene group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkynyl” refers to a monovalent branched or unbranched unsaturated hydrocarbon group preferably having from 2 to 10 carbon atoms and more preferably 2 to 6 carbon atoms and having at least 1 and preferably from 1-2 sites of carbon-carbon triple bond unsaturation. Preferred alkynyl groups include ethynyl (—C≡CH), propargyl (—CH₂C≡CH) and the like.

“Substituted alkynyl” refers to an alkynyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Amino” refers to the group —NH₂.

“Substituted amino” refers to the group —N(R)₂ where each R is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R)₂ is an amino group.

“Alkylamino” refers to the group alkyl-NR′—, wherein R′ is selected from hydrogen and alkyl.

“Arylamino” refers to the group aryl-NR′—, wherein R′ is selected from hydrogen, aryl and heteroaryl.

“Alkoxyamino” refers to a radical —N(R)OR′ where R is selected from hydrogen, alkyl and aryl; and R represents an alkyl or cycloalkyl group as defined herein.

“Alkylarylamino” refers to a radical —NRR′ where R represents an alkyl or cycloalkyl group and R′ is an aryl as defined herein.

“Aminocarbonyl” refers to the group —C(O)NRR where each R is independently hydrogen, alkyl, aryl and cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalkyl, or where the R groups are joined to form an alkylene group.

“Aryl” refers to an unsaturated aromatic carbocyclic group of from 6 to 14 carbon atoms having a single ring (e.g., phenyl) or multiple condensed rings (e.g., naphthyl or anthryl). Preferred aryls include phenyl, biphenyl, naphthyl and the like. Unless otherwise constrained by the definition for the individual substituent, such aryl groups can optionally be substituted with 1 or more substituents, for instance from 1 to 5 substituents, preferably 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Aralkyl” or “arylalkyl” refers to an alkyl group, as defined above, substituted with one or more aryl groups, as defined above.

“Aryloxy” refers to the group —OR where R is aryl.

“Cycloalkyl” refers to a cyclic alkyl group of from 3 to 10 carbon atoms having a single cyclic ring or multiple condensed or bridged rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple or bridged ring structures such as adamantanyl and the like. The term “lower cycloalkyl” refers to a cycloalkyl group having from 3 to 6 carbon atoms.

“Substituted cycloalkyl” refers to a cycloalkyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Cycloalkoxy” refers to the group —OR where R is cycloalkyl. Such cycloalkoxy groups include, by way of example, cyclopentoxy, cyclohexoxy and the like.

“Cycloalkenyl” refers to a cyclic alkenyl group of from 4 to 10 carbon atoms having a single cyclic ring and at least one point of internal unsaturation which can be optionally substituted with from 1 to 3 alkyl groups. Examples of suitable cycloalkenyl groups include, for instance, cyclopent-3-enyl, cyclohex-2-enyl, cyclooct-3-enyl and the like.

“Substituted cycloalkenyl” refers to a cycloalkenyl group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples: 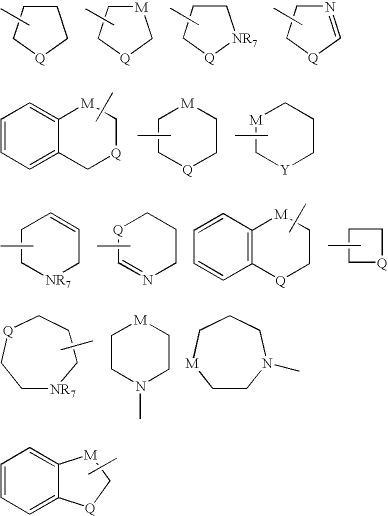
optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives. In the examples, M is CR⁷, NR², O, or S; Q is O, NR² or S. R⁷ and R⁸ are independently selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

As used herein, the term “heteroaryl” refers to an aryl ring system having one to four heteroatoms as ring atoms in a heteroaromatic ring system, wherein the remainder of the atoms are carbon atoms. Suitable heteroatoms include oxygen, sulfur and nitrogen. Preferably, the heterocyclic ring system is monocyclic or bicyclic. Nonlimiting examples include the following, which may be substituted with one or more R⁷: 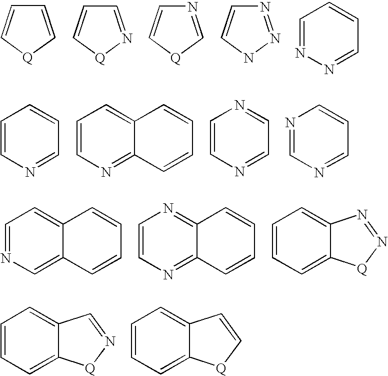
wherein R⁷ and R⁸ are each independently selected from hydrogen, lower alkyl, alkyl, alkenyl, alkynyl, cycloheteroalkyl, alkanoyl, alkoxy, aryloxy, heteroaryloxy, alkylamino, arylamino, heteroarylamino, NR¹¹COR¹², NR¹¹SOₘR¹² where m=1 or 2, COOalkyl, COOaryl, CONR¹¹R¹², CON R¹¹R¹², N R¹¹R¹², SO₂N R¹¹R¹², S(O)n-alkyl or S(O)n-aryl where n is 0, 1 or 2;
R⁷and R⁸ may be joined to form a cyclic ring (saturated or unsaturated) from 5 to 8 atoms, optionally containing one or more heteroatoms selected from the group N, O or S; and
R¹¹, R¹², and R¹² are independently hydrogen, alkyl, alkenyl, alkynyl, perfluoroalkyl, cycloalkyl, cycloheteroalkyl, aryl or heteroaryl;

“Halo” or “halogen” refers to fluoro, chloro, bromo and iodo. Preferred halo groups are either fluoro or chloro.

“Hydroxyl” refers to the group —OH.

“Keto” or “oxo” refers to the group ═O.

“Nitro” refers to the group —NO₂.

“Thioalkoxy” refers to the group —SR where R is alkyl.

“Substituted thioalkoxy” refers to a thioalkoxy group having 1 or more substituents, for instance from 1 to 5 substituents, and preferably from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Sulfanyl” refers to the radical HS—. “Substituted sulfanyl” refers to a radical such as RS— wherein R is any substituent described herein. In certain embodiments, “substituted sulfanyl” refers to a radical —SR where R is an alkyl or cycloalkyl group as defined herein that may be optionally substituted as defined herein. Representative examples include, but are not limited to, methylthio, ethylthio, propylthio, butylthio, and the like.

“Sulfinyl” refers to the radical —S(O)H. “Substituted sulfinyl” refers to a radical such as S(O)—R wherein R is any substituent described herein.

“Sulfonyl” refers to the divalent radical —S(O₂)—. “Substituted sulfonyl” refers to a radical such as —S(O₂)—R wherein R is any substituent described herein. “Aminosulfonyl” or “Sulfonamide” refers to the radical H₂N(O₂)S—, and “substituted aminosulfonyl” “substituted sulfonamide” refers to a radical such as R₂N(O₂)S— wherein each R is independently any substituent described herein. In certain embodiments, R is selected from H, lower alkyl, alkyl, aryl and heteroaryl.

“Thioaryloxy” refers to the group —SR where R is aryl.

“Thioketo” refers to the group ═S.

“Thiol” refers to the group —SH.

The term “subject” refers to an animal such as a mammal, including, but not limited to, primate (e.g., human), cow, sheep, goat, horse, dog, cat, rabbit, rat, mouse and the like. In preferred embodiments, the subject is a human.

The terms “treat,” “treating” or “treatment,” as used herein, refer to a method of alleviating or abrogating a disorder and/or its attendant symptoms. The terms “prevent,” “preventing” or “prevention,” as used herein, refer to a method of barring a subject from acquiring a disorder and/or its attendant symptoms. In certain embodiments, the terms “prevent,” “preventing,” or “prevention,” refer to a method of reducing the risk of acquiring a disorder and/or its attendant symptoms.

“Pharmaceutically acceptable salt” refers to any salt of a compound of this invention which retains its biological properties and which is not biologically or otherwise undesirable. Such salts may be derived from a variety of organic and inorganic counter-ions well known in the art and include, by way of example illustration, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. The term “pharmaceutically-acceptable cation” refers to a pharmaceutically acceptable cationic counter-ion of an acidic functional group. Such cations are exemplified by sodium, potassium, calcium, magnesium, ammonium, tetraalkylammonium cations, and the like.

“Solvate” refers to a compound of the present invention or a salt thereof, that further includes a stoichiometric or non-stoichiometric amount of solvent bound by non-covalent intermolecular forces. Where the solvent is water, the solvate is a hydrate.

The therapeutic methods and pharmaceutical compositions of the invention employ one or more aryl nitrones as the active agent. For the purposes of this invention, the nitrones of formula I are named using conventional nitrone nomenclature, i.e., the carbon atom of the carbon-nitrogen double bond (C═N) is designated the α-position and substituents on the nitrogen atom of the carbon-nitrogen double bond are given the N-prefix.

In some cases, the aryl nitrones of this invention may contain one or more chiral centers. Typically, such compounds will be prepared as a racemic mixture. If desired, however, such compounds can be prepared or isolated as pure stereoisomers, i.e., as individual enantiomers or diastereomers, or as stereoisomer-enriched mixtures. All such stereoisomers (and enriched mixtures) of the aryl nitrones of formula I are included within the scope of this invention. Pure stereoisomers (or enriched mixtures) may be prepared using, for example, optically active starting materials or stereoselective reagents well known in the art. Alternatively, racemic mixtures of such compounds can be separated using, for example, chiral column chromatography, chiral resolving agents and the like.

Additionally, all geometric isomers of the nitrone compounds of formula I are included within the scope of this invention including, for example, all isomers (i.e. E and Z isomers) of the carbon-nitrogen double bond of the nitrone functionality.

As used herein, the term “about” refers to a range of tolerance above or below a quantitative amount known to be acceptable to those of skill in the art. For instance, a dose of about 1000 mg indicates a dose typically administered under the guidance of a practitioner when a dose of 1000 mg is indicated. In certain embodiments, the term “about” refers to ±10% or ±5%.

5.2 2-Substituted Aryl Nitrones of the Invention

The present invention provides 2-substituted aryl nitrones useful for preventing and/or treating diseases and disorders related to oxidative conditions, ischemic conditions and ischemia/reperfusion-related or chemokine mediated conditions in mammals.

In certain embodiments, the present invention provides aryl nitrones according to formula (2.1): 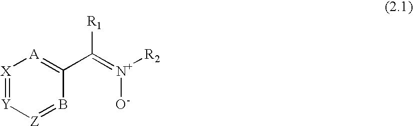
or a pharmaceutically acceptable salt or solvate thereof.

In formula (2.1) R¹ is selected from hydrogen, lower alkyl and alkyl. For example, R¹ can be hydrogen, methyl, ethyl, propyl, butyl and the like. In certain embodiments, R¹ is hydrogen.

R² is selected from lower alkyl, alkyl, aryl, arylalkyl, cycloalkyl, heteroaryl, heteroarylalkyl and cycloheteroalkyl. In certain embodiments, R² is selected from alkyl, aryl, arylalkyl and heteroaryl. In further embodiments, R² is selected from phenyl, benzyl or tert-butyl. Preferred compounds include tert-butyl and benzyl compounds.

At least one of A and B is C—R³, and the other is selected from C—R³ and N. At least one R³is SO₂R⁵, CO₂R⁵, CONR⁵R⁶ or tetrazole, and any other R³ is independently selected from H, lower alkyl, alkenyl, alkyl, halogen, aryl, SO₂R⁵, SO₂NR⁵R⁶, CO₂H, CONR⁵R⁶ and tetrazole. In certain embodiments, each of A and B is independently C—R³.

In certain embodiments, at least one of A and B is C—SO₂R⁵. In further embodiments, at least one of A and B is C—CO₂R⁵. In particular embodiments, at least one of A and B is C—CO₂H. In further embodiments, at least one of A and B is C—CONR⁵R⁶. In further embodiments, at least one of A and B is C-tetrazole.

X, Y and Z are each independently selected from C—R⁴ and N.

In certain embodiments, none of A, B, X, Y and Z are N. In further embodiments, one of A, B, X, Y and Z is N. In further embodiments, two of A, B, X, Y and Z are N. In still further embodiments, three of A, B, X, Y and Z are N. In still further embodiments, four of A, B, X, Y and Z are N.

Each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio. In certain embodiments, each R⁴ is independently selected from H, lower alkyl, alkyl, alkenyl, halogen, aryl, aryloxy, SO₂NR⁵R⁶, SO₂R⁵, CO₂H, CONR⁵R⁶ and tetrazole.

R⁵ and R⁶ are each independently selected from H, lower alkyl, alkyl, aryl and heteroaryl, and where feasible may join together to form a saturated or unsaturated cycloheteroalkyl ring containing 4 to 8 atoms, optionally having one or more heteroatoms selected from NR¹, O and S.

In preferred embodiments, where R³ or R⁴ is SO₂R⁵, R⁵ is not hydrogen.

In a further aspect of the present invention R³ may join with an adjacent R⁴ to form a saturated or un-saturated cyclic ring containing from four to eight atoms, optionally containing one or more heteroatoms selected from the list N, O or S. Thus in this embodiment, compounds of formula (2.2)-(2.4) are provided: 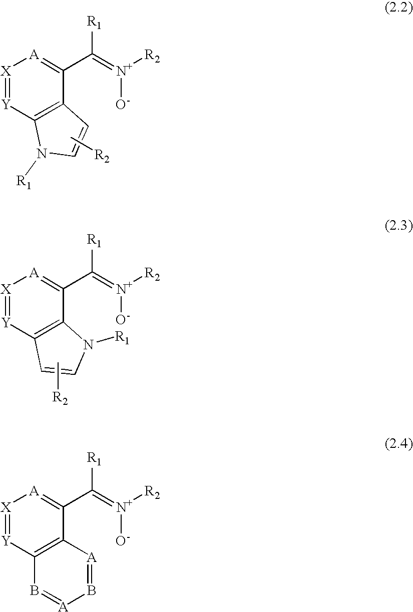
in which the terms R¹, R², R³, R⁴, R⁵, R⁶, A, B, X, Y and Z are as defined above. In certain embodiments, the aryl nitrone compound is a compound according to formula (2.4) wherein the A on the aromatic ring bearing the nitrone group is SO₂R⁵, CO₂R⁵, CONR⁵R⁶ or tetrazole.

In a further aspect of the present invention there is provided a subset of compounds in which two adjacent R⁴ groups may join to form a saturated or un-saturated cyclic ring containing from four to eight atoms, optionally containing one or more heteroatoms selected from the list N, O or S. Thus in this embodiment, compounds of formula (2.5)-(2.6) are provided: 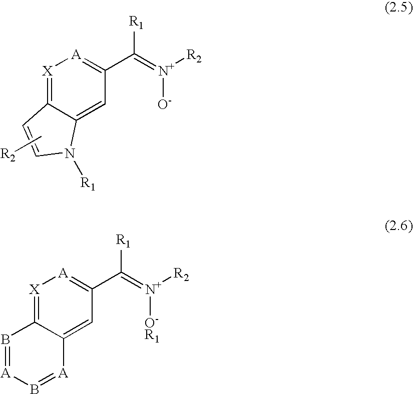
in which the terms R¹, R², R³, R⁴, R⁵, R⁶, A, B, X, Y and Z are as defined above. In certain embodiments, the aryl nitrone compound is a compound according to formula (2.6) wherein the A on the aromatic ring bearing the nitrone group is SO₂R⁵, CO₂R⁵, CONR⁵R⁶ or tetrazole.

In preferred embodiments, the present invention provides compounds according to formula (2.1) wherein the compounds do not include compounds 201-204 below:
201. α-2-carboxy-phenyl-N-t-butyl-nitrone
202. α-2-carboxy-phenyl-N-phenyl-nitrone
203. α-2-carboxy-phenyl-N-3,4-dimethyl-phenyl-nitrone
204. α-2-carboxy-3,4-dimethoxy-phenyl-N-methyl-nitrone
   In certain embodiments, the present invention provides compounds according to any of formulas (2.1)-(2.6) that are not any or all of compounds 201-204, any or all of compounds 2.10-2.210, below, and/or any or all of compounds 1-81 (for instance, any or all of compounds 1-12, 14-16, 62-66, 68, 69 and 72-79) below. In particular embodiments, the present invention provides compounds according to any of formulas (2.1)-(2.6) that are not any of compounds 201-204 or 14 or 15, below.

In further embodiments, the present invention provides individual compounds 201-204, 2.10-2.210 and compounds 1-81 (for instance, compounds 1-12, 14-16, 62-66, 68, 69 and 72-79), pharmaceutically acceptable salts or solvates of these compounds, pharmaceutical compositions comprising these compounds, methods using these compounds and methods of making these compounds as described in detail in the sections below.

In a preferred embodiment of compounds of formula (2.1) to (2.6):
R¹ is selected from H and alkyl,
R² is selected from alkyl, aryl, arylalkyl, cycloalkyl, heteroaryl and heteroarylalkyl,
A, B and R³ are as described above,
X, Y and Z are independently selected from CR⁴ or N,
Each R⁴ is independently selected from H, lower alkyl, alkyl, halogen, aryl, aryloxy, SO₂NR⁵R⁶, SO₂R⁵, CO₂H, CONR⁵R⁶, tetrazole,
R⁵ and R⁶ are each independently selected from H, lower alkyl, alkyl, aryl, heteroaryl, and where feasible may join together to form a saturated or unsaturated cycloheteroalkyl ring containing 4 to 8 atoms, optionally having one or more heteroatoms selected from the list NR¹, O or S.

In an even more preferred embodiment of compounds of formula (2.1) to (2.6):
R¹ is selected from H and alkyl,
R² is selected from alkyl, aryl, arylalkyl, heteroaryl,
A, B and R³ are as described above,
X, Y and Z are independently selected from CR⁴ or N
Each R⁴ is independently selected from H, lower alkyl, alkyl, halogen, aryl, aryloxy, SO₂NR⁵R⁶, SO₂R⁵, CONR⁵R⁶, tetrazole,
R⁵ and R⁶ are each independently selected from H, lower alkyl, alkyl, aryl, heteroaryl, and
   where feasible may join together to form a saturated or unsaturated cycloheteroalkyl ring containing 4 to 8 atoms, optionally having one or more heteroatoms selected from the list NR¹, O or S.

In certain embodiments of compounds of formula (2.1) to (2.6):
R¹ is H; R² is selected from alkyl, aryl, arylalkyl, heteroaryl; at least one R³ is SO₂R⁵, CO₂R⁵, CONR⁵R⁶ or tetrazole; X, Y and Z are independently selected from CR⁴ or N; each R⁴ is independently selected from H, lower alkyl, alkyl, halogen, aryl, aryloxy, SO₂NR⁵R⁶, SO₂R⁵, CONR⁵R⁶, tetrazole; R⁵ and R⁶ are each independently selected from H, lower alkyl, alkyl, aryl, heteroaryl, and where feasible may join together to form a saturated or unsaturated cycloheteroalkyl ring containing 4 to 8 atoms, optionally having one or more heteroatoms selected from the list NR¹, O or S. In certain embodiments according to this paragraph, R² is selected from alkyl and arylalkyl. In further embodiments according to this paragraph, at least one R³ is SO₂R⁵. In further embodiments according to this paragraph, at least one R³ is CO₂R⁵. In further embodiments according to this paragraph, at least one R³ is CONR⁵R⁶. In further embodiments according to this paragraph, at least one R³ is tetrazole. In certain embodiments, R⁵ and R⁶ are each independently H or alkyl or, more particularly H or lower alkyl.

In certain exemplary embodiments, the present invention provides a compound selected from the compounds provided in the examples below and from the following.

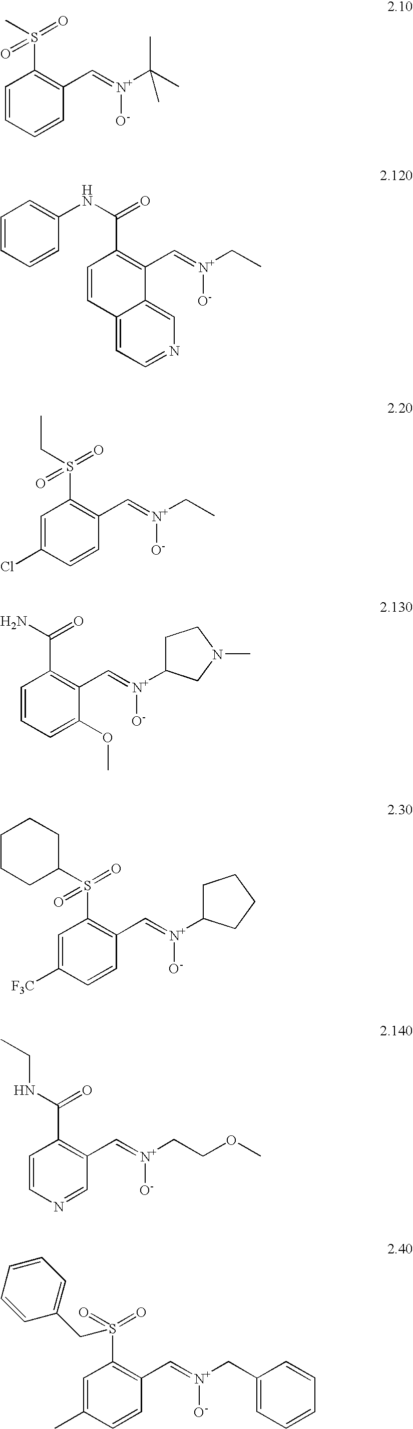

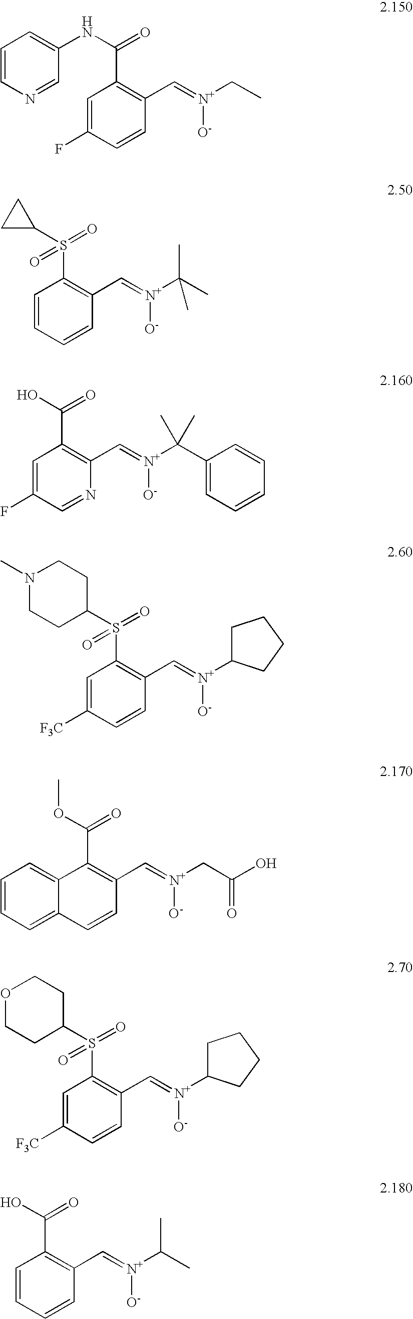

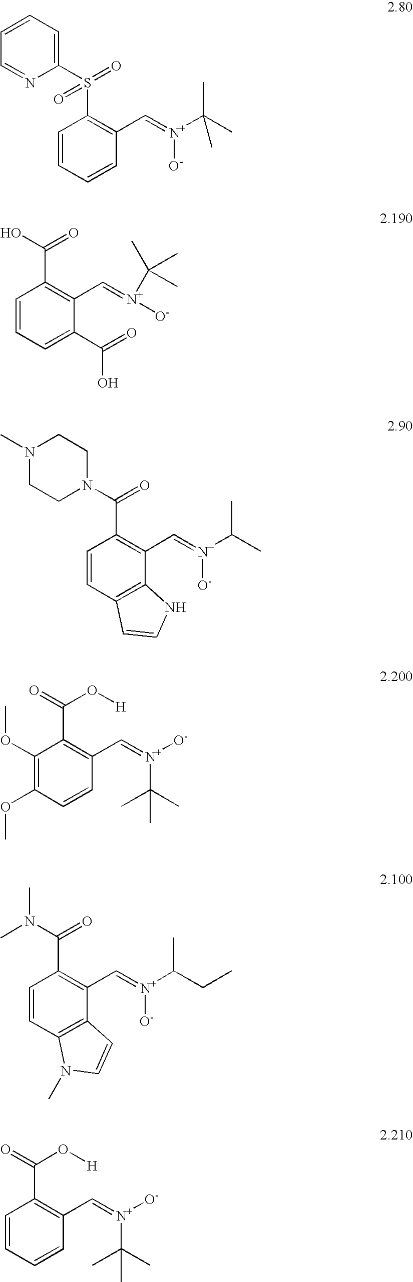

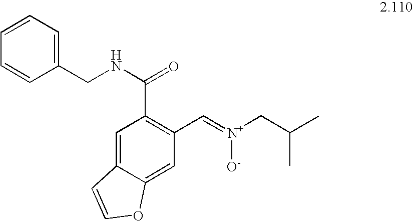

5.3 2-Sulfonamidyl Aryl Nitrones of the Invention

The present invention provides 2-sulfonamidinyl aryl nitrones useful for preventing and/or treating diseases and disorders related to oxidative conditions, ischemic conditions and ischemia/reperfusion-related or chemokine mediated conditions in mammals.

In certain embodiments, the present invention provides aryl nitrones according to formula (3.1): 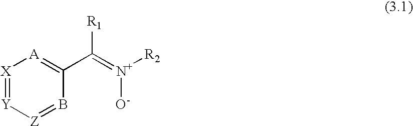
or a pharmaceutically acceptable salt or solvate thereof.

In formula (3.1) R¹ is selected from hydrogen, lower alkyl and alkyl. For example, R¹ can be hydrogen, methyl, ethyl, propyl, butyl and the like. In certain embodiments, R¹ is hydrogen.

R² is selected from lower alkyl, alkyl, aryl, arylalkyl, cycloalkyl, heteroaryl, heteroarylalkyl and cycloheteroalkyl. In certain embodiments, R² is selected from alkyl, aryl, arylalkyl and heteroaryl. In further embodiments, R² is selected from phenyl, benzyl or tert-butyl. Preferred compounds include tert-butyl and benzyl compounds.

At least one of A and B is C—R³, and the other is selected from C—R³ and N. At least one R³ is SO₂NR⁵R⁶, and any other R³ is independently selected from R⁴, H, lower alkyl, alkenyl, alkyl, halogen, aryl, SO₂NR⁵R⁶, SO₂R⁵, CO₂H, CONR⁵R⁶ and tetrazole. In certain embodiments, each of A and B is independently C—R³. In particular embodiments, each of A and B is independently C—R³, and each R³is independently SO₂NR⁵R⁶.

X, Y and Z are each independently selected from C—R⁴ and N.

In certain embodiments, none of A, B, X, Y and Z are N. In further embodiments, one of A, B, X, Y and Z is N. In further embodiments, two of A, B, X, Y and Z are N. In still further embodiments, three of A, B, X, Y and Z are N. In still further embodiments, four of A, B, X, Y and Z are N.

Each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio. In certain embodiments, each R⁴ is independently selected from H, lower alkyl, alkyl, alkenyl, halogen, aryl, aryloxy, SO₂NR⁵R⁶, SO₂R⁵, CO₂H, CONR⁵R⁶ and tetrazole.

R⁵ and R⁶ are each independently selected from H, lower alkyl, alkyl, aryl and heteroaryl, and where feasible may join together to form a saturated or unsaturated cycloheteroalkyl ring containing 4 to 8 atoms, optionally having one or more heteroatoms selected from NR¹, O and S.

In preferred embodiments, where R³ or R⁴ is SO₂R⁵, R⁵ is not hydrogen.

In a further aspect of the present invention R³ may join with an adjacent R⁴ to form a saturated or un-saturated cyclic ring containing from four to eight atoms, optionally containing one or more heteroatoms selected from the list N, O or S. Thus in this embodiment, compounds of formula (3.2)-(3.4) are provided: 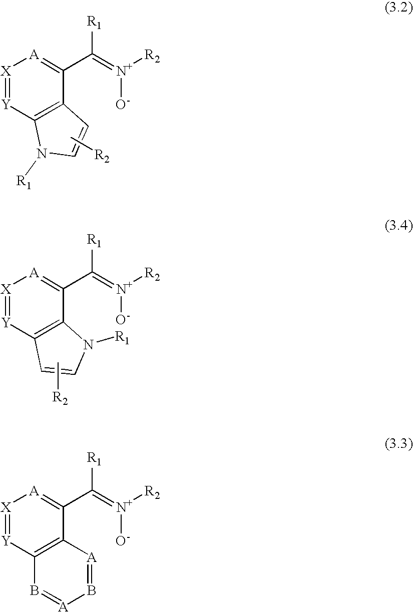
in which the terms R¹, R², R³, R⁴, R⁵, R⁶, A, B, X, Y and Z are as defined above. In certain embodiments, the aryl nitrone compound is a compound according to formula (3.4) wherein the A on the aromatic ring bearing the nitrone group is C—SO₂NR⁵R⁶.

In a further aspect of the present invention there is provided a subset of compounds in which two adjacent R⁴ groups may join to form a saturated or un-saturated cyclic ring containing from four to eight atoms, optionally containing one or more heteroatoms selected from the list N, O or S. Thus in this embodiment, compounds of formula (3.5)-(3.6) are provided: 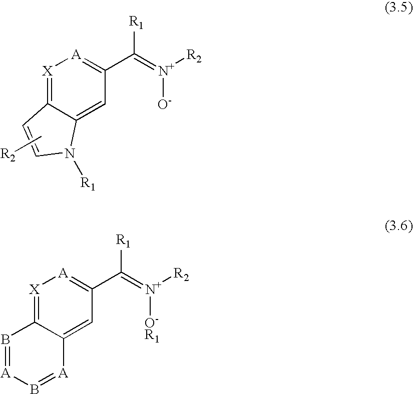
in which the terms R¹, R², R³, R⁴, R⁵, R⁶, A, B, X, Y and Z are as defined above. In certain embodiments, the aryl nitrone compound is a compound according to formula (3.6) wherein the A on the aromatic ring bearing the nitrone group is C—SO₂NR⁵R⁶.

In certain embodiments, the present invention provides compounds according to any of formulas (3.1)-(3.6) that are not any or all of compounds 3.10-3.200, below, and/or any or all of compounds 1-81 (for instance any or all of compounds 13, 18-26, 28-29, 50-61, 63-65, 67, 70, 71, 80 and 81) below.

In further embodiments, the present invention provides individual compounds 3.10-3.200 and compounds 1-81 (for instance compounds 13, 18-26, 28-29, 50-61, 63-65, 67, 70, 71, 80 and 81), pharmaceutically acceptable salts or solvates of these compounds, pharmaceutical compositions comprising these compounds, methods using these compounds and methods of making these compounds as described in detail in the sections below.

In a preferred embodiment of compounds of formula (3.1) to (3.6):
R¹ is selected from H and alkyl,
R² is selected from alkyl, aryl, arylalkyl, cycloalkyl, heteroaryl and heteroarylalkyl,
A, B and R³ are as described above,
X, Y and Z are independently selected from CR⁴ or N,
Each R⁴ is independently selected from H, lower alkyl, alkyl, halogen, aryl, aryloxy, SO₂NR⁵R⁶, SO₂R⁵, CO₂H, CONR⁵R⁶, tetrazole,
R⁵ and R⁶ are each independently selected from H, lower alkyl, alkyl, aryl, heteroaryl, and where feasible may join together to form a saturated or unsaturated cycloheteroalkyl ring containing 4 to 8 atoms, optionally having one or more heteroatoms selected from the list NR¹, O or S.

In an even more preferred embodiment of compounds of formula (3.1) to (3.6):
R¹ is selected from H and alkyl,
R² is selected from alkyl, aryl, arylalkyl, heteroaryl,
A, B and R³ are as described above,
X, Y and Z are independently selected from CR⁴ or N
Each R⁴ is independently selected from H, lower alkyl, alkyl, halogen, aryl, aryloxy, SO₂NR⁵R⁶, SO₂R⁵, CONR⁵R⁶, tetrazole,
R⁵ and R⁶ are each independently selected from H, lower alkyl, alkyl, aryl, heteroaryl, and where feasible may join together to form a saturated or unsaturated cycloheteroalkyl ring containing 4 to 8 atoms, optionally having one or more heteroatoms selected from the list NR¹, O or S.

In certain embodiments of compounds of formula (3.1) to (3.6):
R¹ is H; R² is selected from alkyl, aryl, arylalkyl, heteroaryl; at least one R³ is SO₂NR⁵R⁶;
X, Y and Z are independently selected from CR⁴ or N; each R⁴ is independently selected from H, lower alkyl, alkyl, halogen, aryl, aryloxy, SO₂NR⁵R⁶, SO₂R⁵, CONR⁵R⁶, tetrazole;
R⁵ and R⁶ are each independently selected from H, lower alkyl, alkyl, aryl, heteroaryl, and where feasible may join together to form a saturated or unsaturated cycloheteroalkyl ring containing 4 to 8 atoms, optionally having one or more heteroatoms selected from the list NR¹, O or S. In certain embodiments according to this paragraph, R² is selected from alkyl and arylalkyl. In certain embodiments, R⁵ and R⁶ are each independently H or alkyl or, more particularly H or lower alkyl.

In certain exemplary embodiments, the present invention provides a compound selected from the following:

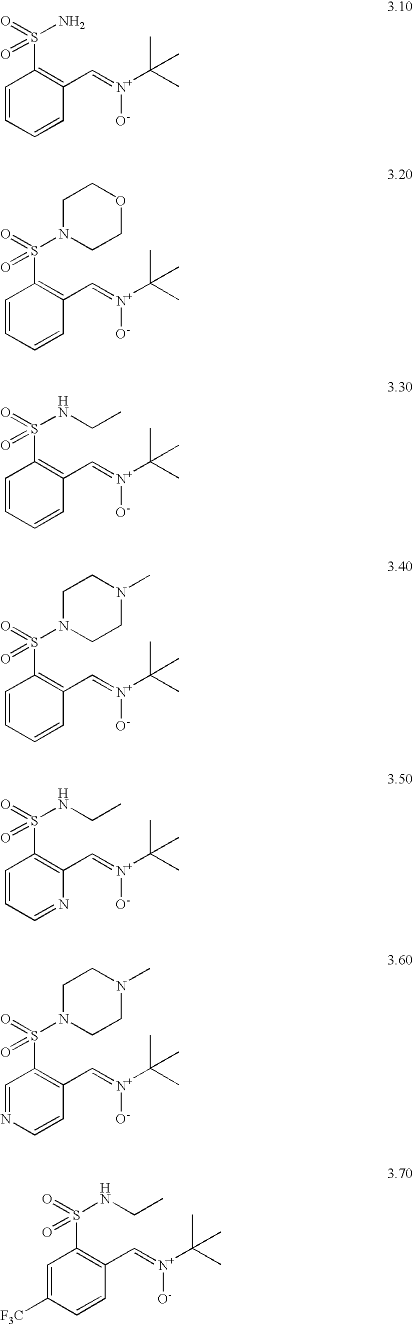

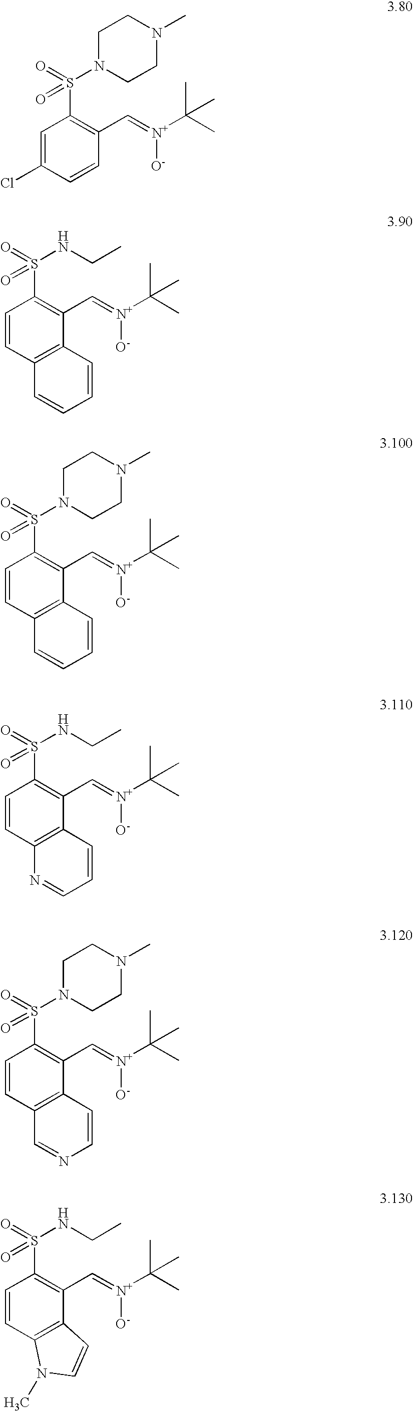

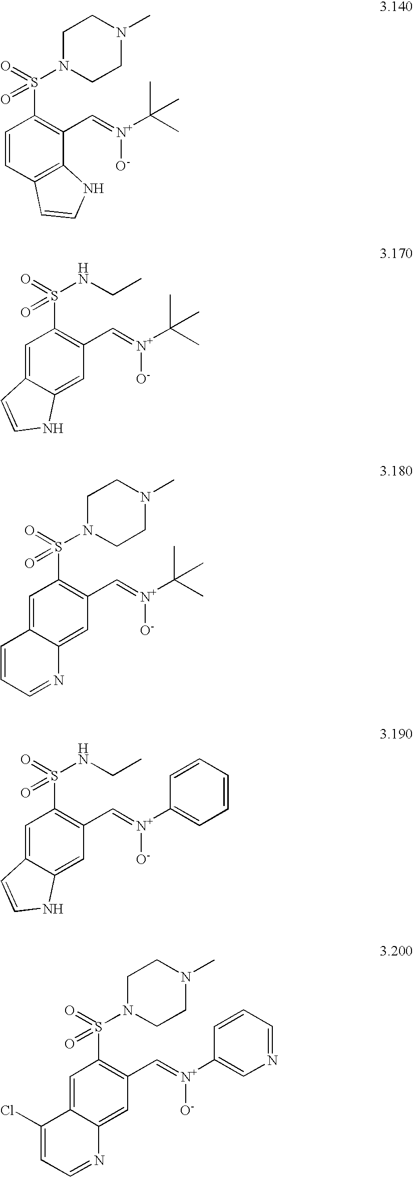

5.4 4-Substituted Aryl Nitrones of the Invention

The present invention provides 4-substituted aryl nitrones useful for preventing and/or treating diseases and disorders related to oxidative conditions, ischemic conditions and ischemia/reperfusion-related or chemokine mediated conditions in mammals.

In certain embodiments, the present invention provides aryl nitrones according to formula (4.1): 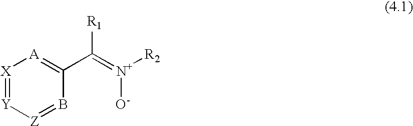
or a pharmaceutically acceptable salt or solvate thereof.
In formula (4.1) R¹ is selected from hydrogen, lower alkyl and alkyl. For example, R¹ can be hydrogen, methyl, ethyl, propyl, butyl and the like. In certain embodiments, R¹ is hydrogen.

R² is selected from lower alkyl, alkyl, aryl, arylalkyl, cycloalkyl, heteroaryl, heteroarylalkyl and cycloheteroalkyl. In certain embodiments, R² is selected from alkyl, aryl, arylalkyl and heteroaryl. In further embodiments, R² is selected from phenyl, benzyl or tert-butyl. Preferred compounds include tert-butyl and benzyl compounds.

Y is C—R⁹, and R⁹ is selected from SO₂NR⁵R⁶, SO₂R⁵, CO₂R⁵, CONR⁵R⁶ and tetrazole. In certain embodiments, Y is C—SO₂R⁵. In further embodiments, Y is C—CO₂R⁵. In particular embodiments, Y is C—CO₂H. In further embodiments, Y is C—CONR⁵R⁶. In further embodiments, Y is C-tetrazole. In preferred embodiments, Y is C—SO₂NR⁵R⁶.

A, B, X and Z are each independently selected from C—R⁴ and N.

In certain embodiments, none of A, B, X, Y and Z are N. In further embodiments, one of A, B, X, Y and Z is N. In further embodiments, two of A, B, X, Y and Z are N. In still further embodiments, three of A, B, X, Y and Z are N. In still further embodiments, four of A, B, X, Y and Z are N.

Each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio. In certain embodiments, each R⁴ is independently selected from H, lower alkyl, alkyl, alkenyl, halogen, aryl, aryloxy, SO₂NR⁵R⁶, SO₂R⁵, CO₂H, CONR⁵R⁶ and tetrazole.

R⁵ and R⁶ are each independently selected from H, lower alkyl, alkyl, aryl and heteroaryl, and where feasible may join together to form a saturated or unsaturated cycloheteroalkyl ring containing 4 to 8 atoms, optionally having one or more heteroatoms selected from NR¹, O and S.

In preferred embodiments, where R³ or R⁴ is SO₂R⁵, R⁵ is not hydrogen.

In a further aspect of the present invention R³ may join with an adjacent R⁴ to form a saturated or un-saturated cyclic ring containing from four to eight atoms, optionally containing one or more heteroatoms selected from the list N, O or S. Thus in this embodiment, compounds of formula (4.2)-(4.4) are provided: 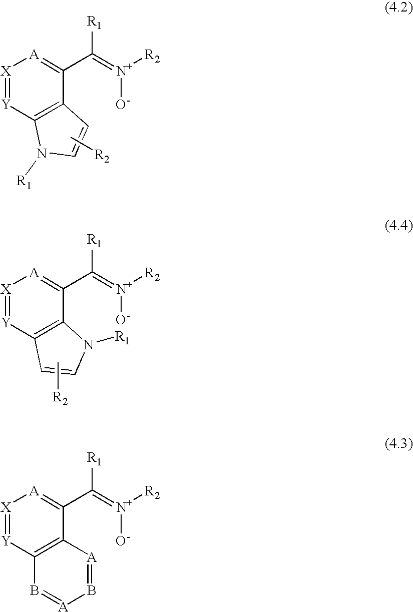
in which the terms R¹, R², R³, R⁴, R⁵, R⁶, A, B, X, Y and Z are as defined above.

In a further aspect of the present invention there is provided a subset of compounds in which two adjacent R⁴ groups may join to form a saturated or un-saturated cyclic ring containing from four to eight atoms, optionally containing one or more heteroatoms selected from the list N, O or S. Thus in this embodiment, compounds of formula (4.5)-(4.6) are provided: 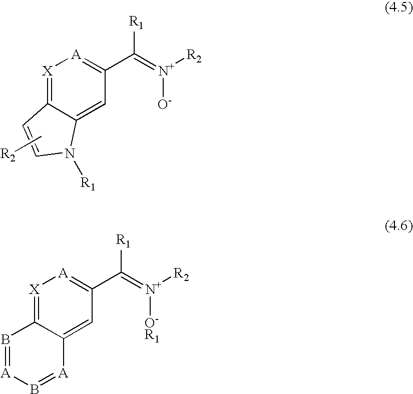
in which the terms R¹, R², R³, R⁴, R⁵, R⁶, A, B, X, Y and Z are as defined above.

In preferred embodiments, the present invention provides compounds according to any of formulas (4.1)-(4.6) wherein the compounds do not include compounds 401-426 below:
401. Benzenamine, N-[[4-(methylsulfonyl)phenyl]methylene]-, N-oxide
402. Benzenamine, 4-bromo-N-[[4-(methylsulfonyl)phenyl]methylene]-, N-oxide
403. Benzenamine, 4-chloro-N-[[4-(methylsulfonyl)phenyl]methylene]-, N-oxide
404. Benzenamine, N-[[4-(methylsulfonyl)phenyl]methylene]-4-nitro-, N-oxide
405. Benzenamine, N-[[4-(methylsulfonyl)phenyl]methylene]-4-(phenylthio)-, N-oxide
406. Benzenamine, N-[[4-(methylsulfonyl)phenyl]methylene]-2-(phenylthio)-, N-oxide
407. Benzenamine, 4-methoxy-N-[[4-(methylsulfonyl)phenyl]methylene]-, N-oxide
408. Phenol, 4-[[[4-(methylsulfonyl)phenyl]methylene]oxidoamino]-
409. Acetamide, N-[4-[[[4-(methylsulfonyl)phenyl]methylene]-oxidoamino]phenyl]-
410. Benzenamine, 4-methyl-N-[[4-(methylsulfonyl)phenyl]methylene]-, N-oxide
411. Benzoic acid, 4-[[(1,1-dimethylethyl)oxidoimino]methyl]-(9CI)
412. Benzoic acid, 4-[[[1,1-dimethyl-2-(octylthio)ethyl]oxidoimino]methyl]-
413. Benzoic acid, 4-[(oxidophenylimino)methyl]; wherein said phenyl group can be para-substituted with alkyl, alkoxy or acyloxy groups containing up to 18 carbon atoms
414. Benzoic acid, 4-[[oxido(5,6,7,8-tetrahydro-5,5,8,8-tetramethyl-2-naphthalenyl)imino]methyl]
415. Benzoic acid, 4-[[(4-ethoxyphenyl)oxidoimino]methyl]
416. Benzoic acid, 4-[[(1,1 -dimethylethyl)oxidoimino]methyl]-2-hydroxy-
417. Benzoic acid, 4-[[oxido(pentamethylphenyl)imino]methyl]-; wherein the ortho and para methyls of said pentamethylphenyl group can be substituted with alkyl or hydrogen
418. Benzamide, N-(1-methylethyl)-4-[[oxido(phenylmethylene)amino]methyl]-
419. Benzamide, 4-[[[[4-[[bis(2,2,6,6-tetramethyl-4-piperidinyl)amino]carbonyl]phenyl]methylene]oxidoamino]methyl]-N,N-bis(2,2,6,6-tetramethyl-4-piperidinyl)-
420. Benzenesulfonamide, 4-[[(1,1-dimethylethyl)oxidoimino]methyl]-
421. Benzenesulfonamide, N-methyl-4-[[oxido(3,4,4-trimethyl-2-thioxo-5-thiazolidinyl)imino]methyl]-
422. Benzenesulfonamide, 4-[[(5,5-dimethyl-3-phenyl-2-thioxo-4-thiazolidinyl)oxidoimino]methyl]-N-methyl-
423. Benzenesulfonamide, N-methyl-4-[[oxido(3,5,5-trimethyl-2-thioxo-4-thiazolidinyl)imino]methyl]-
424. Benzenesulfonamide, 4-[[(3-butyl-5,5-dimethyl-2-thioxo-4-thiazolidinyl)oxidoimino]methyl]- -N-methyl-
425. Benzenesulfonamide, 4-[[(3-propyl-5,5-dimethyl-2-thioxo-4-thiazolidinyl)oxidoimino]methyl]-N-methyl-
426. Benzenesulfonamide, 4-[[(3-phenylmethyl-5,5-dimethyl-2-thioxo-4-thiazolidinyl)oxidoimino]methyl]-N-methyl-
   In certain embodiments, the present invention provides compounds according to any of formulas (4.1)-(4.6) that are not any or all of compounds 401-426, any or all of compounds 4.30-4.280, below, and/or any or all of compounds 1-81 (for instance, any or all of compounds 27 and 30-49) below. In particular embodiments, the present invention provides compounds according to any of formulas (4.1)-(4.6) that are not any of compounds 401-426 or 4.240-4.280 or 13, 18, 19, 20, 21 or 62, below.

In further embodiments, the present invention provides individual compounds 401-426, 4.30-4.280 and compounds 1-81 (for instance, compounds 27 and 30-49), pharmaceutically acceptable salts or solvates of these compounds, pharmaceutical compositions comprising these compounds, methods using these compounds and methods of making these compounds as described in detail in the sections below.

In a preferred embodiment of compounds of formula (4.1) to (4.6):
R¹ is selected from H and alkyl,
R² is selected from alkyl, aryl, arylalkyl, cycloalkyl, heteroaryl and heteroarylalkyl,
Y, A, B and R³ are as described above,
X and Z are independently selected from CR⁴ or N,
Each R⁴ is independently selected from H, lower alkyl, alkyl, halogen, aryl, aryloxy, SO₂NR⁵R⁶, SO₂R⁵, CO₂H, CONR⁵R⁶, tetrazole,
R⁵ and R⁶ are each independently selected from H, lower alkyl, alkyl, aryl, heteroaryl, and where feasible may join together to form a saturated or unsaturated cycloheteroalkyl ring containing 4 to 8 atoms, optionally having one or more heteroatoms selected from the list NR¹, O or S.

In an even more preferred embodiment of compounds of formula (4.1) to (4.6):
R¹ is selected from H and alkyl,
R² is selected from alkyl, aryl, arylalkyl, heteroaryl,
Y, A, B and R³ are as described above,
X and Z are independently selected from CR⁴ or N
Each R⁴ is independently selected from H, lower alkyl, alkyl, halogen, aryl, aryloxy, SO₂NR⁵R⁶, SO₂R⁵, CONR⁵R⁶, tetrazole,
R⁵ and R⁶ are each independently selected from H, lower alkyl, alkyl, aryl, heteroaryl, and
   where feasible may join together to form a saturated or unsaturated cycloheteroalkyl ring containing 4 to 8 atoms, optionally having one or more heteroatoms selected from the list NR¹, O or S.

In certain embodiments of compounds of formula (4.1) to (4.6): R¹ is H; R² is selected from alkyl, aryl, arylalkyl, heteroaryl; R⁹ is selected from SO₂NR⁵R⁶, SO₂R⁵, CO₂R⁵, CONR⁵R⁶ and tetrazole; X, Y and Z are independently selected from CR⁴ or N;

each R⁴ is independently selected from H, lower alkyl, alkyl, halogen, aryl, aryloxy, SO₂NR⁵R⁶, SO₂R⁵, CONR⁵R⁶, tetrazole; R⁵ and R⁶ are each independently selected from H, lower alkyl, alkyl, aryl, heteroaryl, and where feasible may join together to form a saturated or unsaturated cycloheteroalkyl ring containing 4 to 8 atoms, optionally having one or more heteroatoms selected from the list NR¹, O or S. In certain embodiments according to this paragraph, R² is selected from alkyl and arylalkyl. In further embodiments according to this paragraph, R⁹ is SO₂NR⁵R⁶. In further embodiments according to this paragraph, R⁹ is SO₂R⁵. In further embodiments according to this paragraph, R⁹ is CO₂R⁵. In further embodiments according to this paragraph, R⁹ is CONR⁵R⁶. In further embodiments according to this paragraph, R⁹ is tetrazole. In certain embodiments, R⁵ and R⁶ are each independently H or alkyl or, more particularly H or lower alkyl.

In further embodiments of this section, at least one of A and B is independently C—R⁹. In other words, at least one of A and B is substituted with a group selected from SO₂NR⁵R⁶, SO₂R⁵, CO₂R⁵, CONR⁵R⁶ and tetrazole. In particular embodiments, at least one of A and B is substituted with SO₂NR⁵R⁶. In further particular embodiments, at least one of A and B is substituted with SO₂R⁵. In further embodiments of this paragraph, at least one of A and B is C—R⁹ wherein the R⁹ is identical to the R⁹ at Y.

In certain exemplary embodiments, the present invention provides a compound selected from the following or from the compounds provided in the examples below.

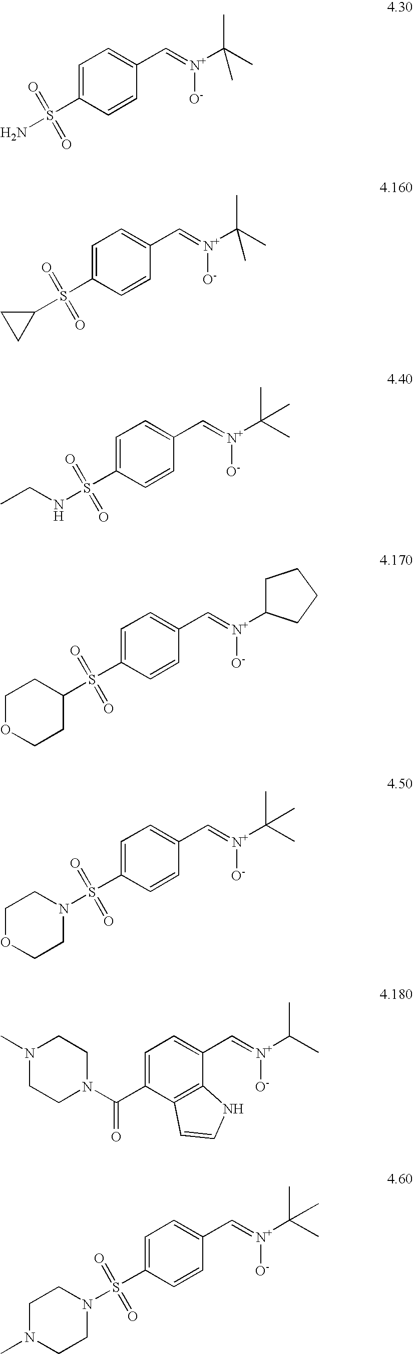

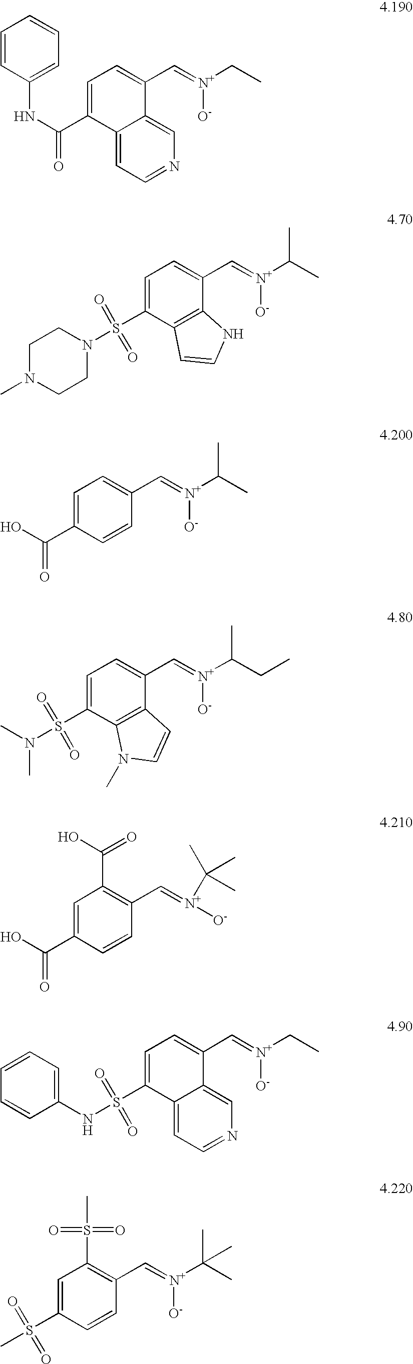

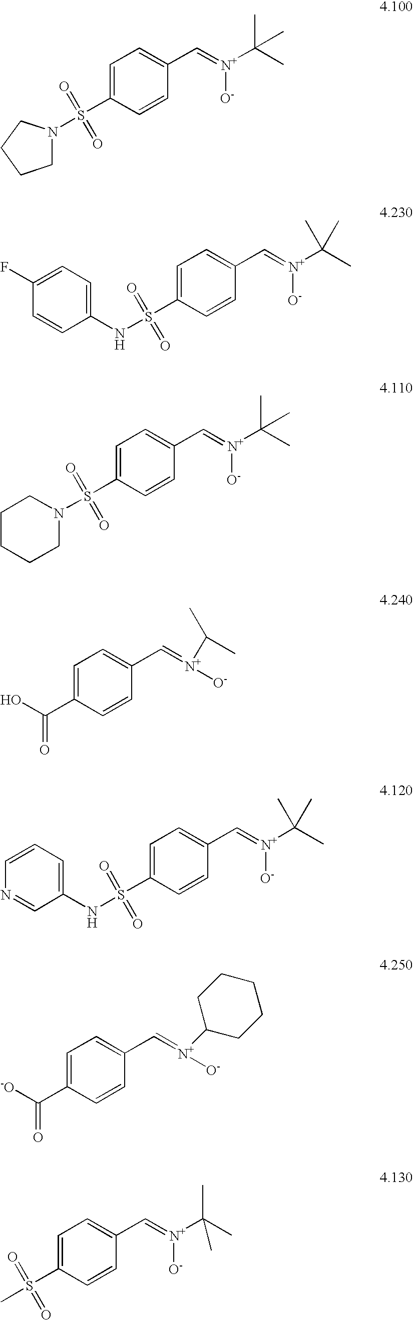

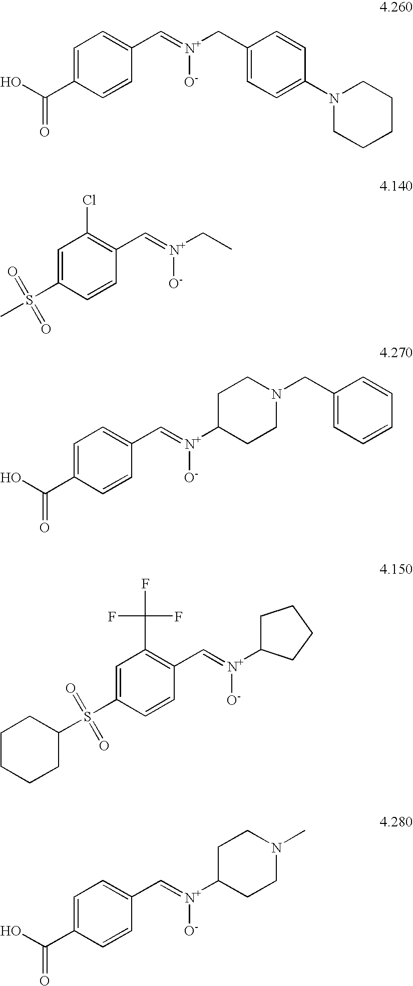

5.5 Substituents of the Nitrone Compounds

While not intending to be bound by any particular theory of operation, the present invention is based, in part, on the discovery that particular substituents at A, B and/or Y yield aryl nitrone compounds with advantageous pharmaceutical properties as illustrated in the examples below. In some embodiments according to (2.1)-(2.6) or (3.1)-(3.6) or (4.1)-(4.6), A or B is C—R³ or Y is C—R⁹ wherein R³ or R⁹ is —SO₂R⁵, —SO₂NR⁵R⁶, —CO₂R⁵, —CONR⁵R⁶ or tetrazole. In certain embodiments, R³ or R⁹ can be selected from —SO₂R⁵ and —SO₂NR⁵R⁶. In further embodiments, R³ or R⁹ is —SO₂R⁶. In further embodiments, R³ or R⁹is —SO₂NR⁷R⁸.

In certain embodiments, the further substituents of the previous paragraph are selected from the substituents described for R⁴ in the paragraphs above. In particular embodiments, the further substituents are selected from the group consisting of hydrogen, alkyl, substituted alkyl, alkoxy, substituted alkoxy, alkoxycarbonyl, substituted alkoxycarbonyl, amino, substituted amino, sulfonyl, substituted sulfonyl, sulfanyl, substituted sulfanyl, aminosulfonyl, substituted aminosulfonyl, carboxy, substituted carboxy (i.e., ester), carbamoyl, substituted carbamoyl, halo, hydroxyl and tetrazole. In more particular embodiments, the further substituents (including R⁴) are selected from the group consisting of hydrogen, lower alkyl, alkyl, alkenyl, halogen, aryl, aryloxy, —SO₂NR⁷R⁸, —SO₃R⁹, —CO₂H, —CO₂R⁹, —CONR⁷R⁸ and tetrazole.

In formulas (2.1)-(2.6) or (3.1)-(3.6) or (4.1)-(4.6), R² is selected from substituted or unsubstituted aliphatic, substituted or unsubstituted heteroaliphatic, substituted or unsubstituted alkyl, substituted or unsubstituted heteroalkyl, substituted or unsubstituted cycloalkyl, substituted or unsubstituted cycloheteroalkyl, substituted or unsubstituted aryl, substituted or unsubstituted heteroaryl, substituted or unsubstituted aralkyl, and substituted or unsubstituted heteroaralkyl. In particular embodiments, R² is alkyl, aryl, arylalkyl, cycloalkyl, heteroaryl and heteroarylalkyl. In more particular embodiments, R² is alkyl or arylalkyl.

In formulas (2.1)-(2.6) or (3.1)-(3.6) or (4.1)-(4.6), R¹ is selected from hydrogen, substituted or unsubstituted (C₁-C₆)alkyl, substituted or unsubstituted (C₁-C₆)cycloalkyl, substituted or unsubstituted aryl, and substituted or unsubstituted aralkyl. In particular embodiments, R¹ is hydrogen or lower alkyl. In more particular embodiments, R¹ is hydrogen.

In formulas (2.1)-(2.6) or (3.1)-(3.6) or (4.1)-(4.6), each R⁵ and R⁶ is independently selected from hydrogen, substituted or unsubstituted aliphatic, substituted or unsubstituted heteroaliphatic, substituted or unsubstituted alkyl, substituted or unsubstituted heteroalkyl, substituted or unsubstituted aryl, substituted or unsubstituted heteroaryl, substituted or unsubstituted aralkyl, substituted or unsubstituted heteroaralkyl, and any adjacent R⁵ and R⁶ may join together to form a substituted or unsubstituted heteroaryl ring or a saturated or unsaturated substituted or unsubstituted cycloheteroalkyl ring of 4 to 7 atoms. In particular embodiments, each R⁵ and R⁶ is independently selected from hydrogen, substituted or unsubstituted aliphatic, substituted or unsubstituted heteroaliphatic, substituted or unsubstituted alkyl, substituted or unsubstituted heteroalkyl and, together, a cycloalkyl ring of 4 to 7 atoms. In certain embodiments, each R⁵ and R⁶ is independently selected from hydrogen, alkyl and, together, a cycloheteroalkyl ring of 4 to 7 atoms. In certain embodiments, R⁵ and R⁶ are each independently H or alkyl or, more particularly H or lower alkyl.

In preferred embodiments of the invention, R² is a substituted carbon. For instance, in certain embodiments, R² is: 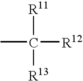
wherein each R¹ , R¹² and R¹³ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, substituted amino, aryl, substituted aryl, arylalkyl, substituted arylalkyl, sulfoxide, substituted sulfoxide, sulfonyl, substituted sulfonyl, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro and thio.

In certain embodiments, at least two of R¹¹, R¹² and R¹³ are other than hydrogen. In further embodiments, all three of R¹¹, R¹² and R¹³ are other than hydrogen.

In certain embodiments, each R¹¹, R¹², and R¹³ is independently selected from lower alkyl, alkyl, aryl, arylalkyl, cycloalkyl, heteroaryl, heteroarylalkyl and cycloheteroalkyl. In further embodiments, each R¹¹, R¹² and R¹³ is independently alkyl or substituted alkyl. In still further embodiments, each R¹¹, R¹² and R¹³ is independently unsubstituted alkyl. In yet further embodiments, each R¹¹, R¹², and R¹³ is independently unsubstituted lower alkyl.

For instance, in certain embodiments, one of R¹¹, R¹² and R¹³ is methyl. In further embodiments, two of R¹¹, R¹² and R¹³ are methyl. In still further embodiments, each of R¹¹, R¹² and R¹³ is methyl.

In particular embodiments, R² is methyl, ethyl, propyl or butyl. For instance, in certain embodiments, R² is isopropyl or tert-butyl.

The present invention also provides compounds according to any combination of the embodiments, preferred embodiments and particular embodiments described above.

Other derivatives of the aryl nitrone compounds of this invention have activity in both their acid and acid-derivative forms. An acid-sensitive form often offers advantages of solubility, tissue compatibility or delayed release in the mammalian organism (See H. Bundgard, 1985, Design of Prodrugs, Elsevier, Amsterdam, pp. 7-9, 21-24). Prodrugs include acid derivatives well known to practitioners of the art, such as, for example, esters prepared by reaction of the parent acid with a suitable alcohol, amides prepared by reaction of the parent acid compound with a substituted or unsubstituted amine, acid anhydrides and mixed anhydrides. Simple aliphatic or aromatic esters, amides and anhydrides derived from acidic groups pendant on the compounds of this invention are preferred prodrugs. In some cases it is desirable to prepare double ester-type prodrugs such as (acyloxy)alkyl esters or ((alkoxycarbonyl)oxy)alkyl esters. Preferred are the C₁-C₈ alkyl, C₂-C₈ alkenyl, aryl, C₇-C₁₂ substituted aryl and C₇-C₁₂ arylalkyl esters of the compounds of the invention.

5.6 Pharmaceutical Compositions

When employed as pharmaceuticals, the aryl nitrones of this invention are typically administered in the form of a pharmaceutical composition. Such compositions can be prepared in a manner well known in the pharmaceutical art and comprise at least one active compound. In preferred embodiments, the active compound is in purified form.

Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of this invention can be administered by a variety of routes including oral, rectal, transdermal, subcutaneous, intravenous, intramuscular, and intranasal. Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the active agent is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the-active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of this invention can also be administered in sustained release forms or from sustained release drug delivery systems. A description of representative sustained release materials can be found in Remington's Pharmaceutical Sciences.

In another embodiment, the pharmaceutical compositions can be in unit dose or unit of use forms or packages. As is known to those of skill in the art, a unit dose form or package is a convenient, prescription size, patient ready unit labeled for direct distribution by health care providers. A unit of use form contains a pharmaceutical composition in an amount necessary for a typical treatment interval and duration for a given indication.

A unit dosage form contains a pharmaceutical composition in an amount necessary for administration of a single dose of the composition. The present invention provides unit dosage forms of pharmaceutical compositions in an amount for delivery of a dose of about 0.1 to 125 mg/kg of the aryl.nitrone to a subject. The subject can be, for example, a human subject with an average weight of about 80 kg. In certain embodiments, the present invention provides a unit dosage form that comprises about 10, 25, 50, 100, 500, 1000, 2000 or 2500 mg of the aryl nitrone. In certain embodiments, the unit dosage form consists essentially of these amounts of the aryl nitrone; in other words, the unit dosage form can additionally comprise other ingredients for administration of the aryl nitrone such as pharmaceutically acceptable carrier, excipient or diluent, a vial, syringe, or patch or other ingredients known to those of skill in the art for administering the aryl nitrone.

Typical unit dosage forms include prefilled, premeasured ampoules or syringes of the injectable compositions or unit dose wrapped tablets or capsules in the case of solid, oral compositions. The unit dosage form can be, for example, a single use vial, a pre-filled syringe, a single transdermal patch and the like

As is known to those of skill in the art, a unit of use form or package is a convenient, prescription size, patient ready unit labeled for direct distribution by health care providers. A unit of use form contains a pharmaceutical composition in an amount necessary for a typical treatment interval and duration for a given indication. The methods of the invention provide for a unit-of-use package of a pharmaceutical composition comprising, for example, an aryl nitrone in an amount sufficient to treat an average sized adult male or female with about 10, 25, 50, 100, 500, 1000, 2000 or 2500 mg orally or 10, 25, 50, 500, 1000, 2000 or 2500 mg subcutaneously three times weekly for one month. Thus a unit of use package as described above would have twelve (three times per week injections for four weeks) prefilled syringes each containing 10, 25, 50, 500, 1000, 2000 or 2500 mg of aryl nitrone pharmaceutical composition.

The pharmaceutical compositions can be labeled and have accompanying labeling to identify the composition contained therein and other information useful to health care providers and subjects in the treatment of the diseases and/or disorders described above, including, but not limited to, instructions for use, dose, dosing interval, duration, indication, contraindications, warnings, precautions, handling and storage instructions and the like.

The following formulation examples illustrate representative pharmaceutical compositions of this invention. The present invention, however, is not limited to the following pharmaceutical compositions.

### Formulation 1—Tablets

A compound of formula I, II or III is admixed as a dry powder with a dry gelatin binder in an approximate 1:2 weight ratio. A minor amount of magnesium stearate is added as a lubricant. The mixture is formed into 240-270 mg tablets (80-90 mg of active amide compound per tablet) in a tablet press.

### Formulation 2—Capsules

A compound of formula I, II or III is admixed as a dry powder with a starch diluent in an approximate 1:1 weight ratio. The mixture is filled into 250 mg capsules (125 mg of active amide compound per capsule).

### Formulation 3—Liquid

A compound of formula I, II or III (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4—Tablets

The compound of formula I, II or III is admixed as a dry powder with a dry gelatin binder in an approximate 1:2 weight ratio. A minor amount of magnesium stearate is added as a lubricant. The mixture is formed into 450-900 mg tablets (150-300 mg of active amide compound) in a tablet press.

### Formulation 5—Injection

The compound of formula I, II or III is dissolved or suspended in a buffered sterile saline injectable aqueous medium to a concentration of approximately 5 mg/ml.

### Formulation 6—Topical

Stearyl alcohol (250 g) and a white petrolatum (250 g) are melted at about 75° C. and then a mixture of a compound of formula I, II or III (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

5.7 Methods Of Treatment and Prevention

The present aryl nitrones are used as therapeutic agents for the treatment of conditions in mammals. Accordingly, the compounds and pharmaceutical compositions of this invention find use as therapeutics for preventing and/or treating oxidative, ischemic, and ischemia/reperfusion-related and chemokine-mediated conditions in mammals including humans. Ischemia and ischemia/reperfusion-related conditions include neurological conditions and cardiovascular conditions as described below.

In a method of treatment or prophylaxis aspect, this invention provides a method of treating or prohpylaxing a mammal susceptible to or afflicted with a neurological condition such as stroke, multi-infarct dementia, traumatic brain injury, spinal cord injury, diabetic neuropathy or neurological sequelae of surgical procedures, which method comprises administering an effective amount of one or more of the pharmaceutical compositions just described. Neurological sequelae of surgical procedures include those sequelae of surgical procedures known to those of skill in the art such as neurological sequelae following procedures using a heart or a lung machine. In particular embodiments, the present invention provides methods of treating or preventing stroke with any compound of the invention.

In yet another method of treatment or prophylaxis aspect, this invention provides a method of treating or prohpylaxing a mammal susceptible to or afflicted with a cardiovascular condition such as myocardial infarction, angina or a non-neurological organ or tissue injury following ischemia, which method comprises administering an effective amount of one or more of the pharmaceutical compositions just described. Non-neurological organ or tissue injury following ischemia include those conditions known to those of skill in the art to follow decreased blood flow or reperfusion following ischemia such as kidney ischemia, muscle ischemia, and the like.

In a further method of treatment or prophylaxis aspect, this invention provides a method of treating or prohpylaxing a mammal susceptible to or afflicted with a condition related to chemokine function such as a neurodegenerative disease, a peripheral neuropathy, an infection, a sequela of an infection, or an autoimmune disease, which method comprises administering an effective amount of one or more of the pharmaceutical compositions just described.

Compounds that inhibit chemokine activity or function may be used for the treatment of diseases that are associated with inflammation, including but not limited to, inflammatory or allergic diseases such as asthma, allergic rhinitis, hypersensitivity lung diseases, hypersensitivity pneumonitis, eosinophilic pneumonias, delayed-type hypersensitivity, interstitial lung disease (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases, such as rheumatoid arthritis, psoriatic arthritis, systemic lupus erythematosus, myastenia gravis, juvenile onset diabetes; glomerulonephritis, autoimmune throiditis, Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Idiopathic Pulmonary Fibrosis, Idiopathic Thrombocytopenia Purpura, IgA Nephropathy, Insulin-dependent Diabetes, Juvenile Arthritis, Lichen Planus, Lupus, Ménière's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, Churg-Strauss Syndrome, Atopic Allergy, Autoimmune Atrophic Gastritis, Achlorhydra Autoimmune, Cushings Syndrome, Dermatomyositis, Erythematosis, Goodpasture's Syndrome, Idiopathic Adrenal Atrophy, Lambert-Eaton Syndrome, Lupoid Hepatitis, Lymphopenia, Phacogenic Uveitis, Primary Sclerosing Cholangitis, Schmidt's Syndrome, Sympathetic Ophthalmia, Systemic Lupus Erythematosis, Thyrotoxicosis, Type B Insulin Resistance, Autoimmune ureitis, Autoimmune oophoritis and orchitis, Dermatitis herpetiformis.graft rejection, including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such as dermatitis, eczema, atopic dermatitis, allergic contact dermatitis, urticaria; vasculitis (e.g., necrotizing, cutaneous, and hypersensitivity vasculitis); eosinphilic myotis, eosiniphilic fasciitis; and cancers.

In addition compounds that activate or promote chemokine receptor function can be used for the treatment of diseases that are associated with immunosuppression such as individuals undergoing chemotherapy, radiation therapy, enhanced wound healing and burn treatment, therapy for autoimmune disease or other drug therapy (e.g., corticosteroid therapy) or combination of conventional drugs used in the treatment of autoimmune diseases and graft/transplantation rejection, which causes immunosuppression; immunosuppression due to congenital deficiency in receptor function or other causes; and infectious diseases, such as parasitic diseases, including but not limited to helminth infections, such as nematodes (round worms); Trichuriasis, Enterobiasis, Ascariasis, Hookworm, Strongyloidiasis, Trichinosis, filariasis; trematodes; visceral worms, visceral larva migtrans (e.g., Toxocara), eosinophilic gastroenteritis (e.g., Anisaki spp., Phocanema ssp.), cutaneous larva migrans (Ancylostona braziliense, Ancylostoma caninum); the malanra-causing protozoan Plasmodium vivax, Human cytomegalovirus, Herpesvirus saimiri, and Kaposi's sarcoma herpesvirus, also known as human herpesvirus 8, and poxyirus Moluscum contagiosum.

In certain embodiments, the present invention provides any compound of the invention for use in the manufacture of a medicament. In further embodiments, the present invention provides any compound of the invention for use in the manufacture of a medicament for the treatment or prevention of any condition identified herein. For instance, the present inveniton provides any compound of the invention for use in the manufacture of a medicament for the treatment and/or prevention of oxidative, ischemic, and ischemia/reperfusion-related and chemokine-mediated conditions in mammals including humans. Such conditions are described in detail herein.

Compounds of the present invention may be used in combination with any other active agents or pharmaceutical compositions where such combined therapy is useful to modulate chemokine receptor activity and thereby prevent and treat inflammatory and immunoregulatory diseases.

Injection dose levels range from about 0.1 mg/kg/hour to at least 15 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 25 g/day for a 40 to 80 kg human patient. The present invention provides doses from about 0. 1 mg to about 25 g per day for an 80 kg human patient. In particular embodiments, the present invention provides doses from about 0.1 mg to about 20 g per day, from about 0.1 mg to about 10 g per day, from about 0.1 mg to about 5 g per day, from about 0.1 mg to about 1 g per day, and from about 0.1 mg to about 0.5 g per day. Preferred doses for ischemic conditions include from about 0.1 mg to about 10 g per day, from about 50 mg to about 10 g per day, from about 100 mg to about 10 g per day, and from about 100 mg to about 1 g per day. Preferred doses for chemokine mediated disorders include from about 0.1 mg to about 10 g per day, from about 10 mg to about 1000 mg per day, and from about 100 mg to about 1000 mg per day.

For the prevention and/or treatment of long-term conditions, such as neurodegenerative and autoimmune conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 65 mg/kg of the aryl nitrone, with preferred doses each providing from about 0.1 to about 20 mg/kg, about 0.1 to about 10 mg/kg and especially about 1 to about 5 mg/kg.

Transdermal doses are generally selected to provide similar or lower blood levels than are achieved using injection doses.

When used to prevent the onset of a neurodegenerative, autoimmune or inflammatory condition, the aryl nitrones of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The compounds of this invention can be administered as the sole active agent or they can be administered in combination with other agents, including other active aryl nitrones.

5.8 Methods of Making the Aryl Nitrones

The aryl nitrones of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

Aryl nitrones of the invention can be prepared, for example, by reaction of an appropriately substituted carboxaldehyde derivative with an appropriately substituted hydroxylamine and the product isolated and purified by known standard procedures. Such procedures include, but are not limited to, recrystallization, column chromatography and HPLC.

Useful starting materials can either be procured from commercial sources or prepared from standard synthetic protocols reported in literature. For instance, 2-formyl phenyl sulfones can be prepared starting from appropriately substituted 2-halo aromatic aldehydes by substitution of the halogen by a sodium sulfide followed by alkylation of the resulting thiol to yield the intermediate thioethers. Controlled oxidation of the thioethers can furnish the desired sulfones.

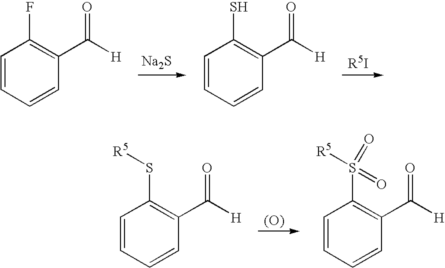

Alternatively, sulfones are accessible starting from 2-halo substituted aromatic aldehydes by nucleophilic substitution by appropriately substituted sodium thiolate followed by oxidation.

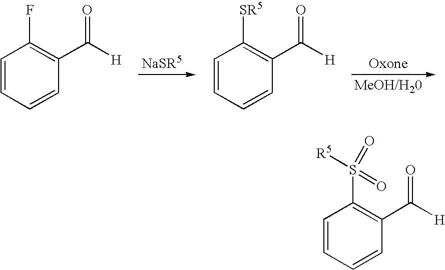

2-formyl carboxamides can be prepared starting from appropriately substituted 2-formyl carboxylic acids by activation of the acid group with either thionyl chloride or POCl₃ followed by reaction with appropriately substituted amine.

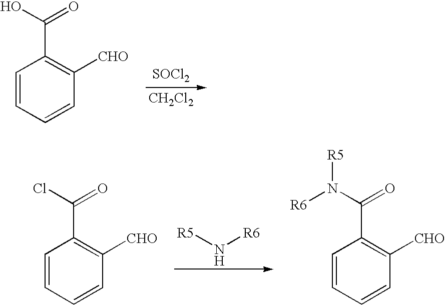

Reaction of an aromatic aldehyde derivative with a substituted hydroxyl amine, in an organic solvent such as methanol, dichloromethane, benzene, toluene or tetrahydrofuran can be used to produce an aromatic nitrone derivative, such as an aryl nitrone of the invention. The reaction can proceed with heating (refluxing), and can proceed with or without an organic or inorganic acid as catalyst. The condensation reaction may also be accomplished using microwave mediated synthesis, and typically employs conditions such as heating to 160° C. for 5 minutes in a sealed tube.

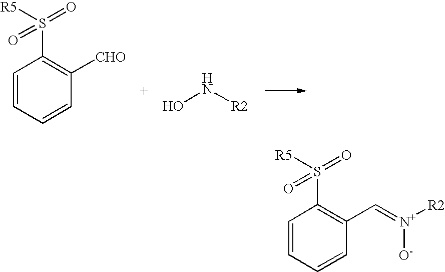

Aryl nitrones of formula (2.1) may also be prepared by alternative well-documented methods such as oxidation of amines, imines, hydroxylamines and N-alkylation of oximes as are known to those of skill in the art and illustrated in the schemes below.

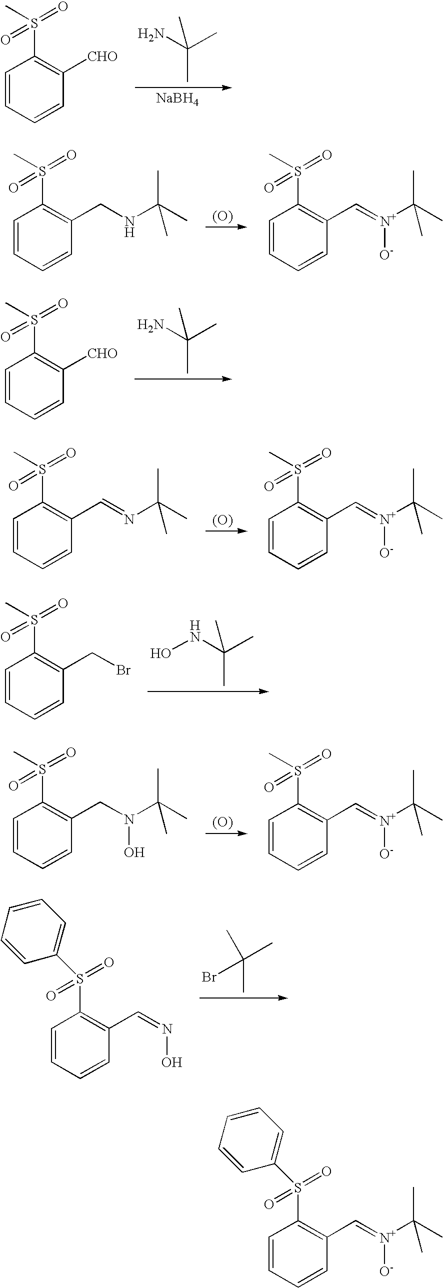

Further, 2-formyl sulfonamides can be prepared starting from appropriately substituted 2-formyl sulfonic acids by activation of the sulfonic acid group with either thionyl chloride or POCL₃ followed by reaction with appropriately substituted amine.

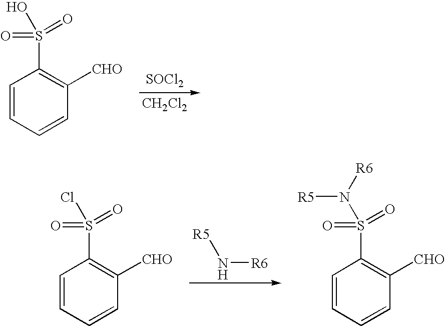

Reaction of an aromatic aldehyde derivative with a substituted hydroxyl amine, in an organic solvent such as methanol, dichloromethane, benzene, toluene or tetrahydrofuran can be used to produce an aromatic nitrone derivative, such as an aryl nitrone of the invention. The reaction can proceed with heating (refluxing), and can proceed with or without an organic or inorganic acid as catalyst. The condensation reaction may also be accomplished using microwave mediated synthesis, and typically employs conditions such as heating to 160 deg for 5 minutes in a sealed tube.

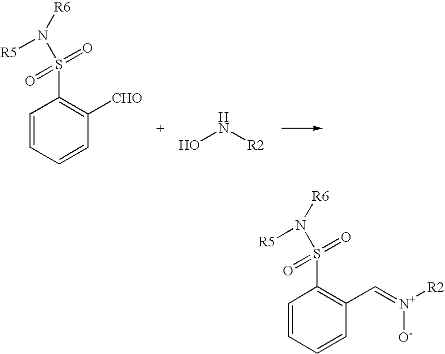

Aryl nitrones of formula (3.1) may also be prepared by alternative well-documented methods such as oxidation of amines, imines, hydroxylamines and N-alkylation of oximes as are known to those of skill in the art and illustrated in the exemplary schemes below.

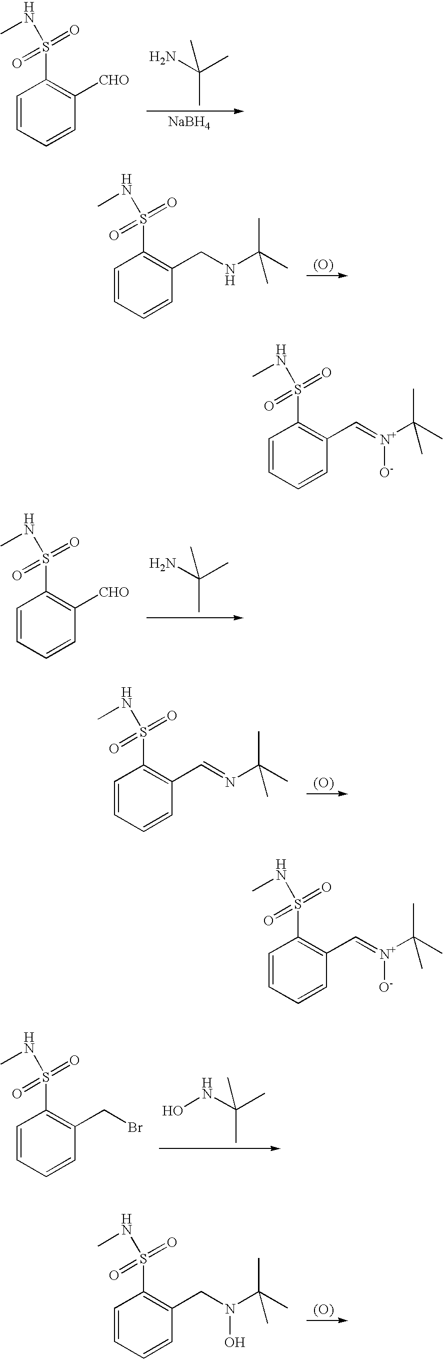

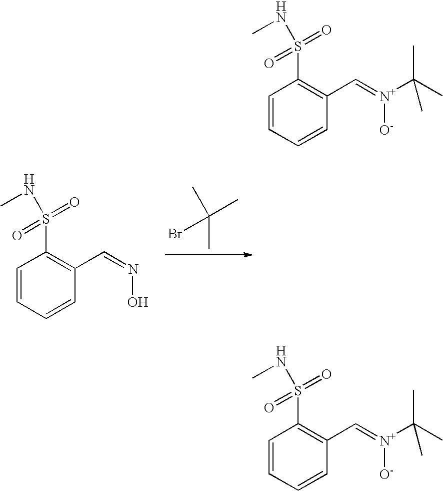

Further, 4-formyl sulfonamides can be prepared from appropriately substituted 4-formyl sulfonyl chloride and reacting with appropriately substituted amines.

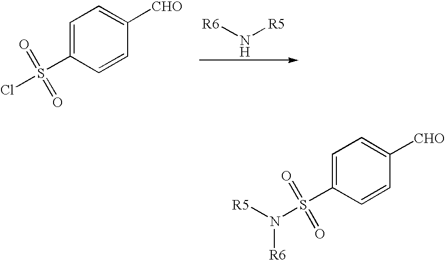

4-formyl phenyl sulfones can be prepared starting from appropriately substituted 4-halo aromatic aldehydes by substitution of the halogen by sodium sulfide followed by alkylation of the resulting thiol to yield the intermediate thioethers. Controlled oxidation of the thioethers furnish the desired sulfones.

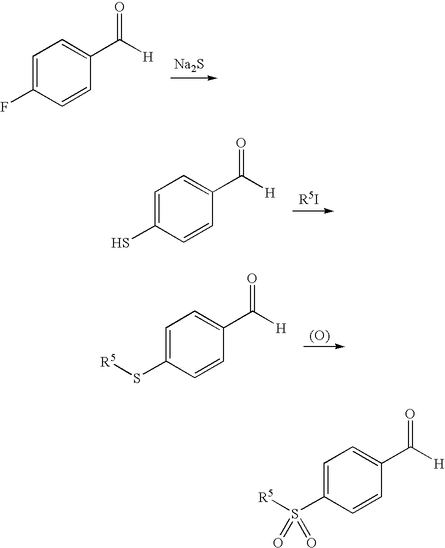

Alternatively, sulfones can be prepared starting from 4-halo substituted aromatic aldehydes by nucleophilic substitution by appropriately substituted sodium thiolates followed by oxidation.

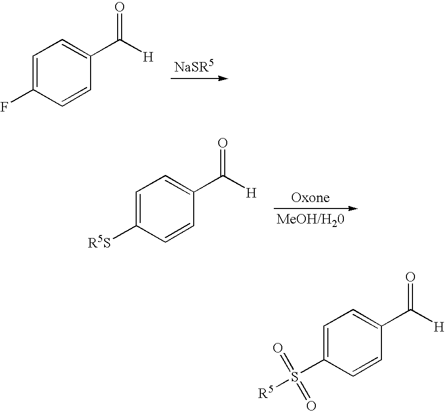

The 4-formyl carboxamides can be prepared starting from appropriately substituted 4-formyl carboxylic acids by activation of the acid group with either thionyl chloride or POCl₃ followed by reaction with appropriately substituted amine.

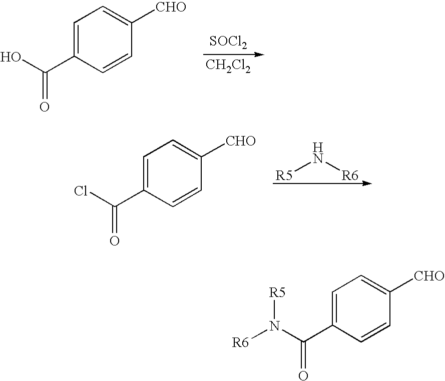

Reaction of an aromatic aldehyde derivative with a substituted hydroxyl amine, in an organic solvent such as methanol, dichloromethane, benzene, toluene or tetrahydrofuran can be used to produce an aromatic nitrone derivative, such as an aryl nitrone of the invention. The reaction can proceed with heating (refluxing), and can proceed with or without an organic or inorganic acid as catalyst. The condensation reaction may also be accomplished using microwave mediated synthesis, and typically employs conditions such as heating to 160 deg for 5 minutes in a sealed tube.

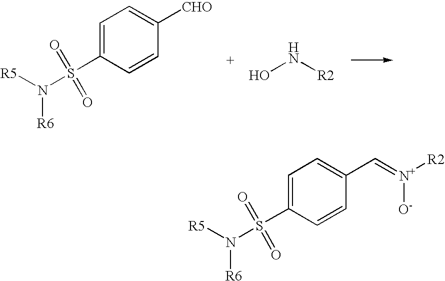

Aryl nitrones of formula (4.1) may also be prepared by alternative well-documented methods such as oxidation of amines, imines, hydroxylamines and N-alkylation of oximes as are known to those of skill in the art and illustrated in the schemes below.

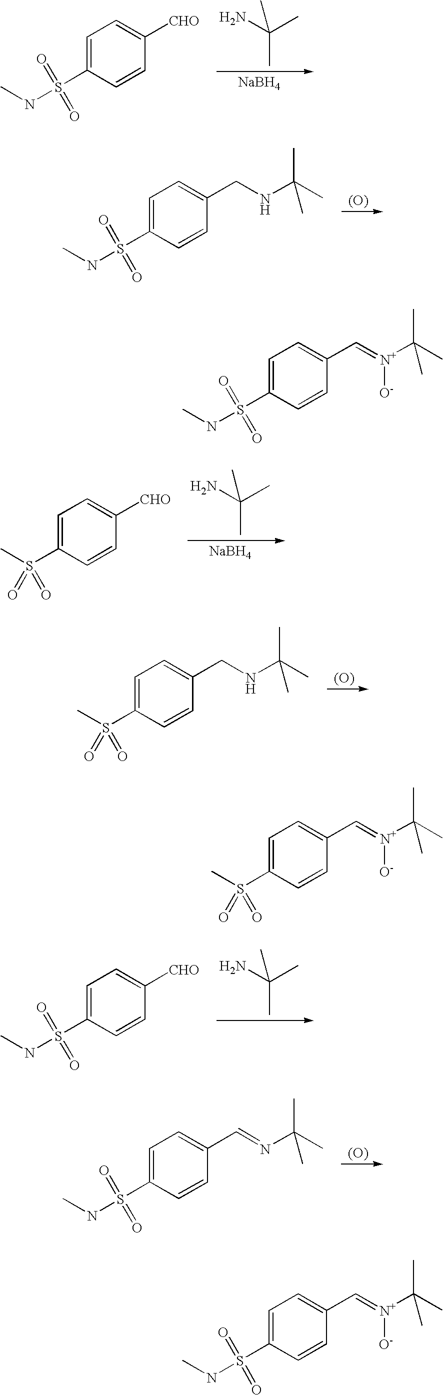

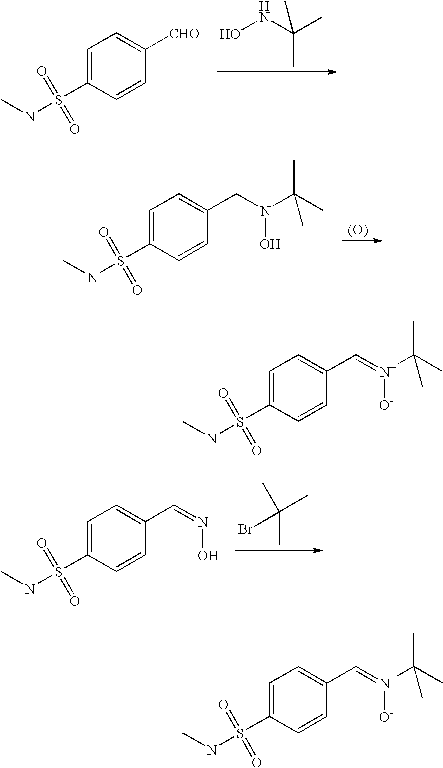

The following synthetic and biological examples are offered to illustrate this invention and are not to be construed in any way as limiting the scope of this invention.

### 6. EXAMPLES

In the examples below, all temperatures are in degrees Celsius unless otherwise indicated. Examples 1-81 describe the synthesis of various aryl, heteroaromatic and bicyclic aryl nitrones of this invention that have been or could be carried out. The graphical depictions of all the nitrone compounds illustrated herein are not intended to indicate the actual (E)- or (Z)-stereochemistry of the C═N double bond of the nitrone group. The present invention provides each stereoisomer of the compounds below.

NMR spectra were recorded at 400 MHz on a JEOL ECX-400 spectrometer employing either deuterated chloroform or DMSO as a solvent and using TMS as internal standard. Chemical shift values are quoted in parts per million (ppm) and coupling constants (J) in hertz (Hz). The FID was transferred to a PC and processed using NUTS® NMR processing software from Acorn NMR, Inc.

### 6.1 Example 1

### N-(tert-Butyl)-C-[2-(methoxycarbonyl)phenyl]nitrone (1)

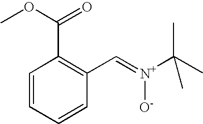

A mixture of commercially available 2-formylbenzoic acid methyl ester (100 mg, 0.61 mmol) and N-(tert-butyl)hydroxylamine hydrochloride (109 mg, 0.732 mmol) in methanol (5 mL) was stirred at ambient temperature for 24 h. The mixture was then concentrated in vacuo and the crude product was dissolved in ethyl acetate (15 ml) and extracted with water (2×20 ml). After the combined organic layers were dried over Na₂SO₄ and concentrated in vacuo, chromatography on silica gel provided compound 1 (10 mg, 20%). MS: m/z 236 (MH+).

Following the procedure described in Example 1, or with slight modifications thereof, and procedures familiar to one of ordinary skill in the art, the compounds of Examples 2-15 were prepared by condensation of appropriate aromatic aldehydes with appropriate hydroxylamines or salts thereof.

### 6.2 Example 2

### N-Cyclohexyl-C-[2-(methoxycarbonyl)phenyl]nitrone (2)

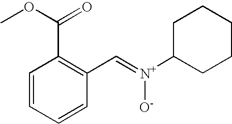

Compound 2 was prepared according to the procedure described in Example 1, starting with N-cyclohexylhydroxylamine hydrochloride and methyl 2-formylbenzoate. MS: m/z 262 (MH+).

### 6.3 Example 3

### N-Benzyl-C-[2-(methoxycarbonyl)phenyl]nitrone (3)

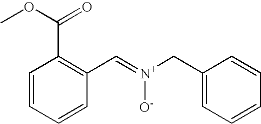

Compound 3 was prepared according to the procedure described in Example 1, starting with N-benzylhydroxylamine hydrochloride and methyl 2-formylbenzoate. MS: m/z 270 (MH+).

### 6.4 Example 4

### N-(tert-Butyl)-C-[2-(methoxycarbonyl)-3,5-dimethoxyphenyl]-nitrone (4)

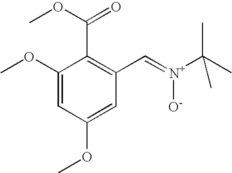

Compound 4 was prepared according to the procedure described in Example 1, starting with N-(tert-butyl)hydroxylamine hydrochloride and methyl 2-formyl-4,6-dimethoxybenzoate. MS: m/z 296 (MH+).

### 6.5 Example 5

### N-Cyclohexyl-C-[2-(methoxycarbonyl)-3,5-dimethoxyphenyl]-nitrone (5)

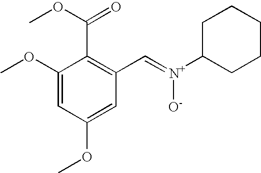

Compound 5 was prepared according to the procedure described in Example 1, starting with N-cyclohexylhydroxylamine hydrochloride and methyl 2-formyl-4,6-dimethoxybenzoate. MS: m/z 322 (MH+).

### 6.6 Example 6

### N-Benzyl-C-[2-(methoxycarbonyl)-3,5-dimethoxyphenyl]-nitrone (6)

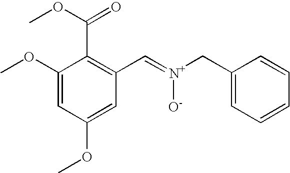

Compound 6 was prepared according to the procedure described in Example 1, starting with N-benzylhydroxylamine hydrochloride and methyl 2-formyl-4,6-dimethoxybenzoate. MS: m/z 330 (MH+).

### 6.7 Example 7

### N-(tert-Butyl)-C-(2-carboxyphenyl)nitrone (7)

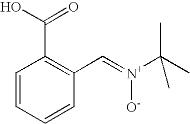

Compound 7 was prepared according to the procedure described in Example 1, starting with N-(tert-butyl)hydroxylamine hydrochloride and 2-formylbenzoic acid. MS: m/z 222 (MH+).

### 6.8 Example 8

### N-Cyclohexyl-C-(2-carboxyphenyl)nitrone (8)

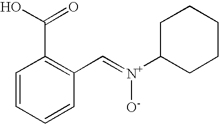

Compound 8 was prepared according to the procedure described in Example 1, starting with N-cyclohexylhydroxylamine hydrochloride and 2-formylbenzoic acid. MS: m/z 248 (MH+).

### 6.9 Example 9

### N-Benzyl-C-(2-carboxyphenyl)nitrone (9)

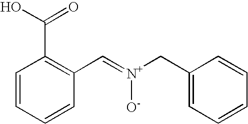

Compound 9 was prepared according to the procedure described in Example 1, starting with N-benzylhydroxylamine hydrochloride and 2-formylbenzoic acid. MS: m/z 256 (MH+).

### 6.10 Example 10

### N-(tert-Butyl)-C-(2-carboxy-3,5-dimethoxyphenyl)nitrone (10)

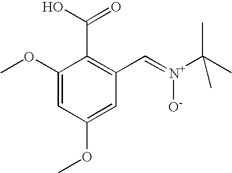

Compound 10 was prepared according to the procedure described in Example 1, starting with N-(tert-butyl)hydroxylamine hydrochloride and 2-formyl-4,6-dimethoxybenzoic acid. MS: m/z 282 (MH+).

### 6.11 Example 11

### N-Cyclohexyl-C-(2-carboxy-3,5-dimethoxyphenyl)nitrone (11)

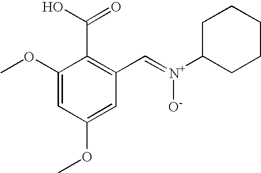

Compound 11 was prepared according to the procedure described in Example 1, starting with N-cyclohexylhydroxylamine hydrochloride and 2-formyl-4,6-dimethoxybenzoic acid. MS: m/z 308 (MH+).

### 6.12 Example 12

### N-Benzyl-C-(2-carboxy-3,5-dimethoxyphenyl)nitrone (12)

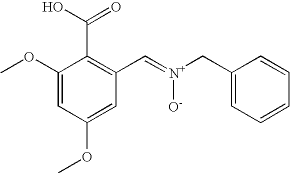

Compound 12 was prepared according to the procedure described in Example 1, starting with N-benzylhydroxylamine hydrochloride and 2-formyl-4,6-dimethoxybenzoic acid. MS: m/z 316 (MH+).

### 6.13 Example 13

### N-tert-Butyl-C-(4-carboxy-phenyl)nitrone (13)

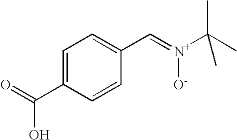

Compound 13 was prepared according to the procedure described in Example 1, starting with N-tert-butylhydroxylamine hydrochloride and 4-formylbenzoic acid. MS: m/z 222 (MH+).

### 6.14 Example 14

### N-tert-Butyl-C-(2-carboxy-phenyl)nitrone (14)

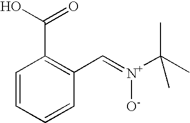

Compound 14 was prepared according to the procedure described in Example 1, starting with N-tert-butylhydroxylamine hydrochloride and 2-formylbenzoic acid. MS: m/z 222 (MH+).

### 6.15 Example 15

### N-tert-Butyl-C-(2-carboxy-3,5-dimethoxyphenyl)nitrone (15)

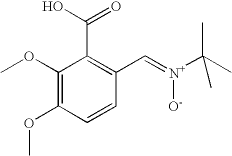

Compound 15 was prepared according to the procedure described in Example 1, starting with N-tert-butylhydroxylamine hydrochloride and 6-formyl-2,3-dimethoxy benzoic acid. MS: m/z 282 (MH+).

### 6.16 Example 16

### N-(tert-Butyl)-C-[2-(N,N-dimethylcarbamoyl)phenyl]nitrone (16)

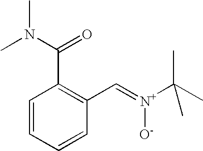

(a) 2-Formyl-N,N-dimethylbenzamide

To a suspension of 2-carboxybenzaldehyde (500 mg, 3.33 mmol) in CH₂Cl₂ (25 ml) was added thionyl chloride (1.98 g, 16.65 mmol) and the mixture was refluxed for 1 h. The resulting solution was then concentrated in vacuo, dissolved in THF, and treated with N,N-dimethylamine (3.9 ml of a 1 M solution in THF, 180 mg, 4.0 mmol) at ice-cold temperature. The mixture was warmed slowly to ambient temperature and stirred at ambient temperature for 2 h. The mixture was then concentrated in vacuo and the crude product was subjected to flash chromatography on silica gel to provide 2-formyl-N,N-dimethylbenzamide (100 mg, 15%). MS: m/z 178 (MH+).

(b) N-(tert-Butyl)-C-[2-(N,N-dimethylcarbamoyl)phenyl]nitrone (16)

Compound 16 was prepared by condensing 2-formyl-N,N-dimethylbenzamide with N-(tert-butyl)hydroxylamine hydrochloride according to the procedure described in Example 1. MS: m/z 249 (MH+).

### 6.18 Example 18

### N-(tert-Butyl)-C-[4-(N-tert-butylcarbamoyl)phenyl]nitrone (18)

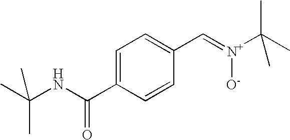

Compound 18 was prepared according to the procedure described in Example 16, starting with N-tert-butylhydroxylamine hydrochloride and 4-formylbenzoic acid. MS: m/z 277 (MH+).

### 6.19 Example 19

### N-(tert-Butyl)-C-[4-(aminocarbamoyl)phenyl]nitrone (19)

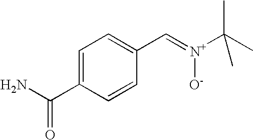

Compound 19 was prepared according to the procedure described in Example 16, starting with N-tert-butylhydroxylamine hydrochloride and 4-formylbenzoic acid. MS: m/z 221 (MH+).

### 6.20 Example 20

### N-(tert-Butyl)-C-[4-(sulfamoyl)phenyl]nitrone (20)

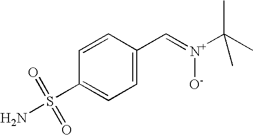

A suspension of 4-formylbenzenesulfonic acid sodium salt (1.0 g, 4.78 mM) in excess of thionylchloride (15 ml) was heated to reflux for 30 minutes. The mixture was then concentrated to dryness, dissolved in anhydrous THF (20 ml). The mixture was cooled (ice-bath) to which was added excess of ammonia (5 ml, 1.0M solution in THF) and the suspension was stirred for 3 hrs at ambient temperature. The reaction was quenched with ice-cold water where up on the amide precipitated out. It was filtered, washed with water and vacuum dried overnight. Efforts were not made to purify the amide and it was used as such in the subsequent reaction.

The crude amide was dissolved in methanol (20 ml) and subjected to condensation with N-tert-butylhydroxylamine hydrochloride (0.72 g, 5.74 mM) at refluxing temperature for 6 hrs. Concentration of the mixture followed by silicagel column chromatography afforded the title compound as a white solid. MS: m/z 257 (MH+). ¹H NMR δ 1.51(s, 9H); 7.39(brs, 2H); 7.83(d, J=8.8 Hz, 2H); 7.99(s, 1H); 8.49 (d, J=8.8 Hz, 2H).

Following the procedure described in Example 20, or with slight modifications thereof, and procedures familiar to one of ordinary skill in the art, the compounds of Examples 21-61 were prepared by condensation of appropriate aromatic aldehydes with appropriate hydroxylamines or salts thereof.

### 6.21 Example 21

### N-(tert-Butyl)-C-[4-(3-methoxy-phenylsulfamoyl)phenyl]nitrone (21)

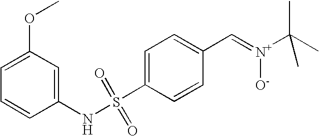

The title compound was prepared according to the procedure described in 20, starting from N-tert-butylhydroxylamine hydrochloride and 4-(3-methoxy phenylsulfamoyl)benzaldehyde MS: m/z 363 (MH+).

### 6.22 Example 22

### N-(tert-Butyl)-C-[4-(4-methyl-piperazine-1-sulfonyl)phenyl]nitrone (22)

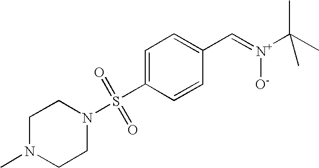

The title compound was prepared according to the procedure described in Example 20, starting from N-tert-butylhydroxylamine hydrochloride and 4-(4-methyl-piperazine-1-sulfonyl)benzaldehyde MS: m/z 340 (MH+).

### 6.23 Example 23

### N-(tert-Butyl)-C-[4-(morpholine-4-sulfonyl)phenyl]nitrone (23)

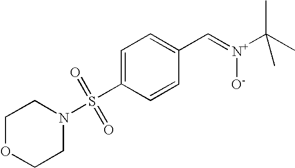

a) 4-(Morpholine-4-sulfonyl)-benzaldehyde:

Morpholine (8.94 g, 102.62 mM; 2.1 eq.) was slowly dropped into a cooled (0° C.) solution of the 4-formylbenzene sulfonyl chloride (10.0 g, 48.87 mM; 1.0 eq.) and the mixture was slowly warmed to ambient temperature. TLC indicated complete disappearance of the starting sulfonyl chloride. The mixture was then poured on to ice-cold water, the solid was filtered, washed with water and vacuum dried to obtain the title sulfonamide as an off-white solid (11.5 g, 92%). The purity read 98% by LC/MS.

b) N-(tert-Butyl)-C-[4-(morpholine-4-sulfonyl)phenyl]nitrone (23)

A mixture of 4-(morpholine-4-sulfonyl)-benzaldehyde (11.5 g, 45.05 mM; 1.0 eq.) and tert-butylhydroxylamine acetate (8.07 g, 54.06 mM; 1.2 eq.) in methanol was refluxed for several hrs (monitored by TLC for the disappearance of the starting aldehyde). The mixture was then concentrated, dissolved in EtOAc, washed with water (to eliminate hydroxylamine acetate), dried and concentrated. The crude product was crystallized from EtOAc/hexane to obtain the title nitrone (11.0 g, 75%) as an off-white solid. MS: m/z 327 (MH+). ¹H NMR δ 1.52 (s, 9H); 2.86(t, J=4.6 Hz, 4H); 3.62(t, J=4.6 Hz, 4H); 7.76(d, J=8.8 Hz, 2H); 8.08(s, 1H); 8.59(d, J=8.8 Hz, 2H).

### 6.24 Example 24

### N-(tert-Butyl)-C-[4-(ethylsulfamoyl)phenyl]nitrone (24)

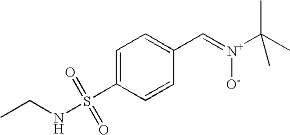

The title compound was prepared according to the procedure described in Example 23, starting from N-tert-butylhydroxylamine hydrochloride and 4-(ethylsulfamoyl) benzaldehyde MS: m/z 285 (MH+).

### 6.25 Example 25

### N-(tert-Butyl)-C-[4-(4-fluoro-phenylsulfamoyl)phenyl]nitrone (25)

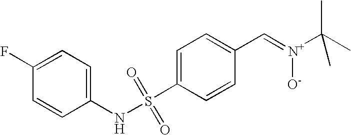

The title compound was prepared according to the procedure described in Example 23, starting from N-tert-butylhydroxylamine hydrochloride and 4-(4-fluorophenylsulfamoyl) benzaldehyde MS: m/z 351 (MH+).

### 6.26 Example 26

### N-(tert-Butyl)-C-[4-(pyridin-3-ylsulfamoyl)phenyl]nitrone (26)

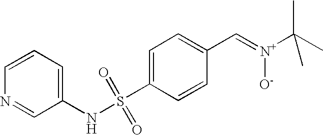

The title compound was prepared according to the procedure described in Example 23, starting from N-tert-butylhydroxylamine hydrochloride and 4-(pyridin-3-ylsulfamoyl) benzaldehyde MS: m/z 334 (MH+).

### 6.27 Example 27

### N-(tert-Butyl)-C-[4-(morpholine-4-sulfonyl)phenyl]nitrone (27)

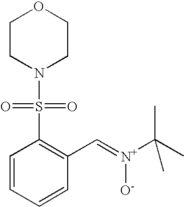

The title compound was prepared according to the procedure described in Example 23, starting from N-tert-butylhydroxylamine hydrochloride and 2-(morpholine-4-sulfonyl) benzaldehyde MS: m/z 327 (MH+).

### 6.28 Example 28

### N-(tert-Butyl)-C-[4-(piperidine-1-sulfonyl)phenyl]nitrone (28)

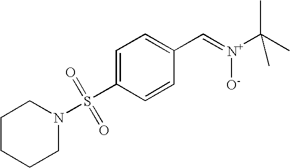

The title compound was prepared according to the procedure described in Example 23, starting from N-tert-butylhydroxylamine hydrochloride and 4-(piperidine-1-sulfonyl) benzaldehyde MS: m/z 325 (MH+).

### 6.29 Example 29

### N-(tert-Butyl)-C-[4-(pyrrolidine-1-sulfonyl)phenyl]nitrone (29)

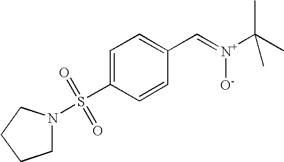

The title compound was prepared according to the procedure described in Example 23, starting from N-tert-butylhydroxylamine hydrochloride and 4-(pyrrolidine-1-sulfonyl) benzaldehyde MS: m/z 311 (MH+).

### 6.30 Example 30

### N-tert-Butyl-C-(2-diethylsulfamoylphenyl)nitrone (30)

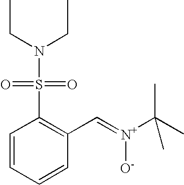

The title compound was prepared according to the procedure described in Example 23, starting from N-tert-butylhydroxylamine hydrochloride and 2-(diethylsulfamoyl) benzaldehyde MS: m/z 313 (MH+).

### 6.31 Example 31

### N-Cyclohexyl-C-(2-diethylsulfamoylphenyl)nitrone (31)

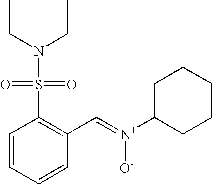

The title compound was prepared according to the procedure described in Example 23, starting from N-cyclohexylhydroxylamine hydrochloride and 2-(diethylsulfamoyl) benzaldehyde MS: m/z 339 (MH+).

### 6.32 Example 32

### N-Benzyl-C-(2-diethylsulfamoylphenyl)nitrone (32)

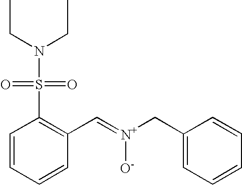

The title compound was prepared according to the procedure described in Example 23, starting from N-benzylhydroxylamine hydrochloride and 2-(diethylsulfamoyl) benzaldehyde MS: m/z 347 (MH+).

### 6.33 Example 33

### N-tert-Butyl-C-[2-(piperidine-1-sulfonyl)phenyl]nitrone (33)

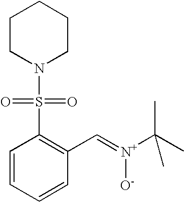

The title compound was prepared according to the procedure described in Example 23, starting from N-tert-butylhydroxylamine hydrochloride and 2-(piperidine-1-sulfonyl) benzaldehyde MS: m/z 325 (MH+).

### 6.34 Example 34

### N-Cyclohexyl-C-[2-(piperidine-1-sulfonyl)phenyl]nitrone (34)

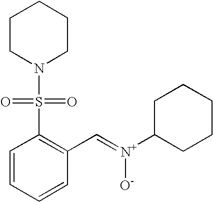

The title compound was prepared according to the procedure described in Example 23, starting from N-cyclohexylhydroxylamine hydrochloride and 2-(piperidine-1-sulfonyl) benzaldehyde MS: m/z 351 (MH+).

### 6.35 Example 35

### N-Benzyl-C-[2-(piperidine-1-sulfonyl)phenyl]nitrone (35)

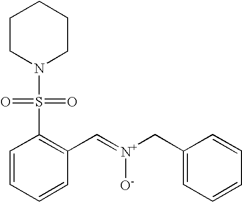

The title compound was prepared according to the procedure described in Example 23, starting from N-benzylhydroxylamine hydrochloride and 2-(piperidine-1-sulfonyl) benzaldehyde MS: m/z 359 (MH+).

### 6.36 Example 36

### N-Cyclohexyl-C-[2-(morpholine-4-sulfonyl))phenyl]nitrone (36)

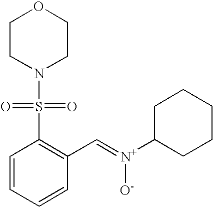

The title compound was prepared according to the procedure described in Example 23 starting from N-cyclohexylhydroxylamine hydrochloride and 2-(morpholine-4-sulfonyl) benzaldehyde MS: m/z 353 (MH+).

### 6.37 Example 37

### N-Benzyl-C-[2-(morpholine-4-sulfonyl))phenyl]nitrone (37)

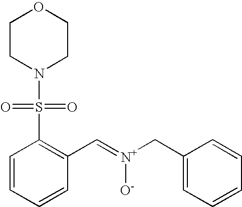

The title compound was prepared according to the procedure described in Example 23, starting from N-benzylhydroxylamine hydrochloride and 2-(morpholine-4-sulfonyl) benzaldehyde MS: m/z 361 (MH+).

### 6.38 Example 38

### N-tert-Butyl-C-[2-(4-methyl-piperazine-1-sulfonyl)phenyl]nitrone (38)

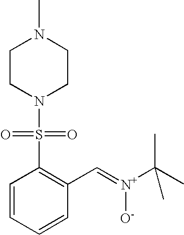

The title compound was prepared according to the procedure described in Example 23, starting from N-tert-butylhydroxylamine hydrochloride and 2-(4-methyl-piperazine-1-sulfonyl) benzaldehyde MS: m/z 340 (MH+). ¹H NMR δ 1.51(s, 9H); 2.13(s, 3H); 2.32(t, J=4.7 Hz, 4H); 2.94(t, J=4.7 Hz, 4H); 7.63 (dt, J=7.7 Hz, 1.4 Hz, 1H); 7.76(dt, J=7.7 Hz, 1.0 Hz, 1H); 8.20(dd, J=7.9 Hz, 1.4 Hz, 1H); 8.46(s, 1H); 9.16(dd, J=7.9 Hz, 1.0 Hz, 1H).

### 6.39 Example 39

### N-Cyclohexyl-C-[2-(4-methyl-piperazine-1-sulfonyl)phenyl]nitrone (39)

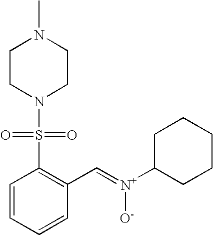

The title compound was prepared according to the procedure described in Example 23, starting from N-cyclohexylhydroxylamine hydrochloride and 2-(4-methyl-piperazine-1-sulfonyl) benzaldehyde MS: m/z 366 (MH+).

### 6.40 Example 40

### N-Benzyl-C-[2-(4-methyl-piperazine-1-sulfonyl)phenyl]nitrone (40)

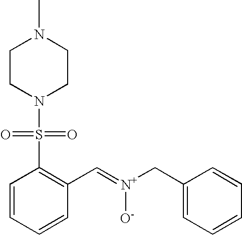

The title compound was prepared according to the procedure described in Example 23, starting from N-benzylhydroxylamine hydrochloride and 2-(4-methyl-piperazine-1-sulfonyl) benzaldehyde MS: m/z 374 (MH+). ¹H NMR δ 1.23(s, 9H); 3.12(s, 3H); 7.10-7.14(m, 2H); 7.29-7.40(m, 3H); 7.61(dt, J=7.8 Hz, 1.4 Hz, 1H); 7.75(dt, J=7.8 Hz, 1.4 Hz, 1H); 7.91(dd, J=8.1 Hz, 1.4 Hz, 1H); 8.01(s, 1H); 9.39(dd, J=8.1 Hz, 1H).

### 6.41 Example 41

### N-tert-Butyl-C-[4-(2-methyl-phenyl-sulfamoyl)phenyl]nitrone (41)

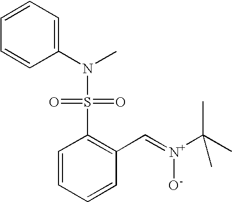

The title compound was prepared according to the procedure described in Example 23, starting from N-tert-butylhydroxylamine hydrochloride and 2-(2-methyl-phenyl-sulfamoyl) benzaldehyde MS: m/z 347 (MH+).

### 6.42 Example 42

### N-Cyclohexyl-C-[4-(2-methyl-phenyl-sulfamoyl)phenyl]nitrone (42)

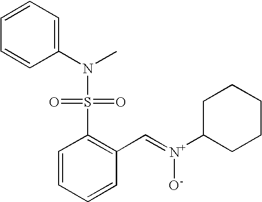

The title compound was prepared according to the procedure described in Example 23, starting from N-cyclohexylhydroxylamine hydrochloride and 2-(2-methyl-phenyl-sulfamoyl) benzaldehyde MS: m/z 373 (MH+).

### 6.43 Example 43

### N-Benzyl-C-[4-(2-methyl-phenyl-sulfamoyl)phenyl]nitrone (43)

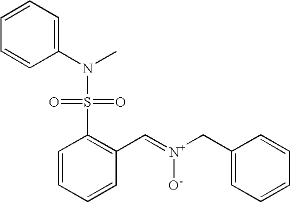

The title compound was prepared according to the procedure described in Example 23, starting from N-benzylhydroxylamine hydrochloride and 2-(2-methyl-phenyl-sulfamoyl) benzaldehyde MS: m/z 381 (MH+).

### 6.44 Example 44

### N-tert-Butyl-C-[2-(3,4-dihydro-2H-quinoline-1-sulfonyl)phenyl]nitrone (44)

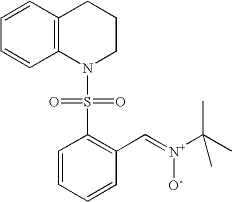

The title compound was prepared according to the procedure described in Example 23, starting from N-benzylhydroxylanine hydrochloride and 2-(3,4-dihydro-2H-quinoline-1-sulfonyl) benzaldehyde MS: m/z 373 (MH+).

### 6.45 Example 45

### N-Cyclohexyl-C-[2-(3,4-dihydro-2H-quinoline-1-sulfonyl)phenyl]nitrone (45)

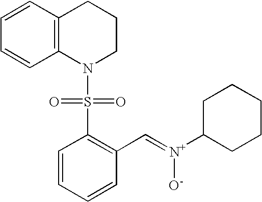

The title compound was prepared according to the procedure described in Example 23, starting from N-cyclohexylhydroxylamine hydrochloride and 2-(3,4-dihydro-2H-quinoline-1-sulfonyl) benzaldehyde MS: m/z 399 (MH+).

### 6.46 Example 46

### N-Benzyl-C-[2-(3,4-dihydro-2H-quinoline-1-sulfonyl)phenyl]nitrone (46)

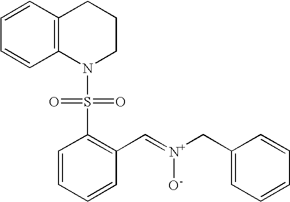

The title compound was prepared according to the procedure described in Example 23, starting from N-benzylhydroxylamine hydrochloride and 2-(3,4-dihydro-2H-quinoline-1-sulfonyl) benzaldehyde MS: m/z 407 (MH+).

### 6.47 Example 47

### N-tert-Butyl-C-(2-methylsulfamoyl-phenyl)nitrone (47)

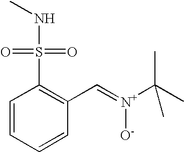

The title compound was prepared according to the procedure described in Example 23, starting from N-tert-butylhydroxylamine hydrochloride and 2-(2-methylsulfamoyl) benzaldehyde MS: m/z 271 (MH+).

### 6.48 Example 48

### N-tert-Butyl-C-(2-tert-butylsulfamoyl-phenyl)nitrone (48)

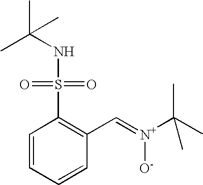

The title compound was prepared according to the procedure described in Example 23, starting from N-tert-butylhydroxylamine hydrochloride and 2-(2-tert-butylsulfamoyl) benzaldehyde MS: m/z 313 (MH+).

### 6.49 Example 49

### N-tert-Butyl-C-(2-benzylsulfamoyl-phenyl)nitrone (49)

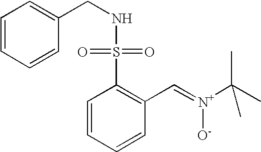

The title compound was prepared according to the procedure described in Example 23, starting from N-tert-butylhydroxylamine hydrochloride and 2-(2-benzylsulfamoyl) benzaldehyde MS: m/z 347 (MH+).

### 6.50 Example 50

### N-tert-Butyl-C-(4-diethylsulfamoylphenyl)nitrone (50)

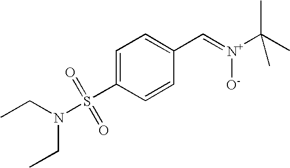

The title compound was prepared according to the procedure described in Example 23, starting from N-tert-butylhydroxylamine hydrochloride and 4-(diethylsulfamoyl) benzaldehyde MS: m/z 313 (MH+).

### 6.51 Example 51

### N-Isopropyl-C-(4-diethylsulfamoylphenyl)nitrone (51)

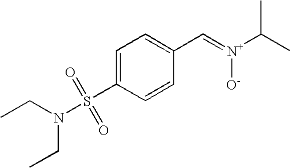

The title compound was prepared according to the procedure described in Example 23, starting from N-isopropylhydroxylamine hydrochloride and 4-(diethylsulfamoyl) benzaldehyde MS: m/z 299 (MH+).

### 6.52 Example 52

### N-Cyclohexyl-C-(4-diethylsulfamoylphenyl)nitrone (52)

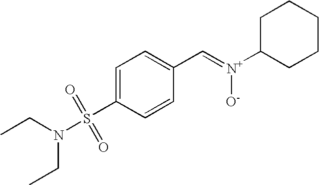

The title compound was prepared according to the procedure described in Example 23, starting from N-cyclohexylhydroxylamine hydrochloride and 4-(diethylsulfamoyl) benzaldehyde MS: m/z 339 (MH+).

### 6.53 Example 53

### N-Benzyl-C-(4-diethylsulfamoylphenyl)nitrone (53)

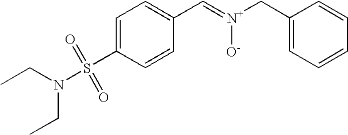

The title compound was prepared according to the procedure described in Example 23, starting from N-benzylhydroxylamine hydrochloride and 4-(diethylsulfamoyl) benzaldehyde MS: m/z 347 (MH+).

### 6.54 Example 54

### N-tert-Butyl-C-[4-(methyl-phenyl-sulfamoyl)phenyl]nitrone (54)

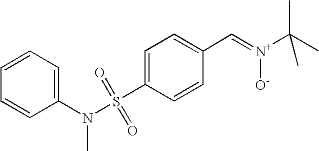

The title compound was prepared according to the procedure described in Example 23, starting from N-tert-butylhydroxylamine hydrochloride and 4-(methyl-phenyl-sulfamoyl) benzaldehyde MS: m/z 347 (MH+). ¹H NMR δ 1.51(s, 9H); 3.14(s, 3H); 7.06-7.12(m, 2H); 7.26-7.36(m, 3H); 7.51(d, J=8.8 Hz, 2H); 8.03(s, 1H); 8.49(d, J=8.8 Hz, 2H).

### 6.55 Example 55

### N-Isopropyl-C-[4-(methyl-phenyl-sulfamoyl)phenyl]nitrone (55)

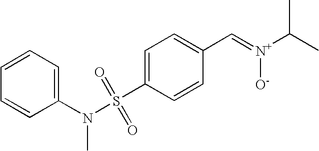

The title compound was prepared according to the procedure described in Example 23, starting from N-isopropylhydroxylamine hydrochloride and 4-(methyl-phenyl-sulfamoyl) benzaldehyde MS: m/z 333 (MH+).

### 6.56 Example 56

### N-Cyclohexyl-C-[4-(methyl-phenyl-sulfamoyl)phenyl]nitrone (56)

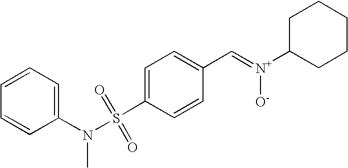

The title compound was prepared according to the procedure described in Example 23, starting from N-cyclohexylhydroxylamine hydrochloride and 4-(methyl-phenyl-sulfamoyl) benzaldehyde MS: m/z 373 (MH+).

### 6.57 Example 57

### N-Benzyl-C-[4-(methyl-phenyl-sulfamoyl)phenyl]nitrone (57)

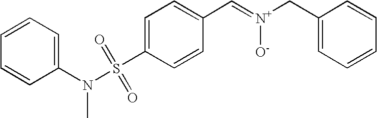

The title compound was prepared according to the procedure described in Example 23, starting from N-benzylhydroxylamine hydrochloride and 4-(methyl-phenyl-sulfamoyl) benzaldehyde MS: m/z 381 (MH+).

### 6.58 Example 58

### N-tert-Butyl-C-[4-(tert-butylsulfamoyl)phenyl]nitrone (58)

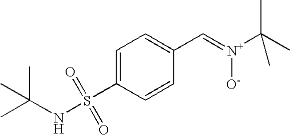

The title compound was prepared according to the procedure described in Example 23, starting from N-tert-butylhydroxylamine hydrochloride and 4-(tert-butylsulfamoyl) benzaldehyde MS: m/z 313 (MH+). ¹H NMR δ 1.08(s, 9H); 1.56(s, 9H); 7.56(s, 1H); 7.83(d, J=8.8 Hz, 2H); 8.0(s, 1H); 8.49(d, J=8.8 Hz, 2H).

### 6.59 Example 59

### N-Isopropyl-C-[4-(tert-butylsulfamoyl)phenyl]nitrone (59)

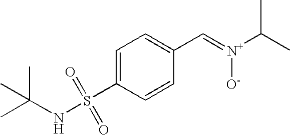

The title compound was prepared according to the procedure described in Example 23, starting from N-isopropylhydroxylamine hydrochloride and 4-(tert-butylsulfamoyl) benzaldehyde MS: m/z 299 (MH+).

### 6.60 Example 60

### N-Cyclohexyl-C-[4-(tert-butylsulfamoyl)phenyl]nitrone (60)

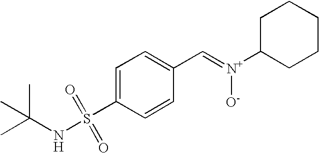

The title compound was prepared according to the procedure described in Example 23, starting from N-cyclohexylhydroxylamine hydrochloride and 4-(tert-butylsulfamoyl) benzaldehyde MS: m/z 339 (MH+).

### 6.61 Example 61

### N-Benzyl-C-[4-(tert-butylsulfamoyl)phenyl]nitrone (61)

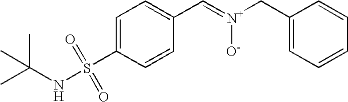

The title compound was prepared according to the procedure described in Example 23, starting from N-benzylhydroxylamine hydrochloride and 4-(tert-butylsulfamoyl) benzaldehyde. MS: m/z 347 (MH+).

### 6.62 Example 62

### N-tert-Butyl-C-[4-(methanesulfonyl)phenyl]nitrone (62)

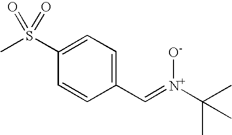

a) N-tert-Butyl-C-[4-(methanesulfanyl)phenyl]nitrone

A mixture of 4-methylsulfanyl benzaldehyde (47.0 g, 0.315 M) and tert-butylhydroxyl amine acetate (40.0 g, 0.263 M) in methanol (300 ml) was refluxed overnight. After all the starting aldehyde has disappeared (monitored by TLC), the mixture was concentrated to dryness. The crude product was dissolved in EtOAc, washed with sat. NaHCO₃ solution followed by water, the organic layer was dried and concentrated to obtain the title product as an oil (51.0 g, 87%). The purity read >95% and it was subjected to oxidation without further purification.

b) N-tert-Butyl-C-[4-(methanesulfonyl)phenyl]nitrone (62)

Oxone (160.0 g, 0.26 M) in EDTA (4×10⁻⁴ in 400 ml water) solution was slowly added during 15 minutes at 0° C. to suspension of the nitrone (51.0 g, 0.228 M) and NaHCO₃ (110.0 g, 1.31 M) in a mixture of acetone (150 ml) and water (150 ml). The mixture was stirred at the same temperature for an additional 2 hrs before being partitioned between EtOAc and water. The organic layer was separated, washed with water, dried and concentrated. The crude product was chromatographed on silicagel to obtain the title product (25.0 g, 43%) as a white solid. MS: m/z 256 (MH+). ¹H NMR δ 1.52(s, 9H); 3.22(s, 3H); 7.95(d, J=8.8 Hz, 2H); 8.06(s, 1H); 8.57(d, J=8.8 Hz, 2H).

Following the procedure described in Example 62, or with slight modifications thereof, and procedures familiar to one of ordinary skill in the art, the compounds of Examples 63-76 were prepared by condensation of appropriate aromatic aldehydes with appropriate hydroxylamines or salts thereof.

### 6.63 Example 63

### N-tert-Butyl-C-(2,4-bis-methanesulfonylphenyl)nitrone (63)

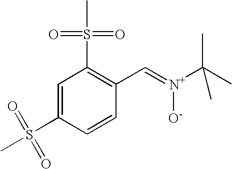

a) 2,4-bis-Methylsulfanyl-benzaldehyde

Sodium thiomethoxide (12 g, 171 mmol) was suspended in DMF (80 mL). To the mixture a solution of 2, 4-diflorobenzaldehyde (8.9 mL, 82 mmol) in DMF (20 mL) was added dropwise at 0° C. The mixture was then stirred at room temperature for ˜3 hrs. The yellow crystals were precipitated out from the solution while H₂O was added. The crystals were collected via filtration and were washed with H₂O and vacuum dried to obtain the title compound (Yield: 13.9 g, yellow crystals).

b) N-tert-Butyl-C-(2,4-bis-methanesulfanylphenyl)nitrone

A mixture of 2,4-bis-methylsulfanyl benzaldehyde (18.0 g, 90.31 mM) and tert-butylhydroxyl amine acetate (16.25 g, 108.92 mM) in methanol (200 ml) was refluxed overnight. After all the starting aldehyde has disappeared (monitored by TLC), the mixture was concentrated to dryness. The crude product was dissolved in EtOAc, washed with sat. NaHCO₃ solution followed by water, the organic layer was dried and concentrated and chromatographed on silicagel to obtain of the title product (24.0 g) as an oil.

c). N-tert-Butyl-C-(2,4-bis-methanesulfonylphenyl)nitrone (63)

Oxone (149.0 g, 142 mM; 5.4 eq.) in EDTA (4×10⁻⁴ in 200 ml water,) solution was slowly added during 15 minutes at 0° C. to a suspension of the above sulfanyl nitrone (12.07 g, 44.8 mM; 1.0 eq.) and NaHCO₃ (90.3 g, 1.08 M; 22.5 eq.) in a mixture of acetone (100 ml) and water (100 ml). The mixture was stirred at the same temperature for an additional 2 hrs before being partitioned between EtOAc and water. The organic layer was separated, washed with water, dried and concentrated. The crude product was crystallized from EtOAC/hexane to obtain the title sulfonyl product (10.0 g, 68%) as a white solid. MS: m/z 334 (MH+). ¹H NMR δ 1.55(s, 9H); 3.33(s, 6H); 8.33(dd, J=8.5 Hz, 1.8 Hz, 1H); 8.45(d, J=1.8 Hz, 1H); 8.67(s, 1H); 9.46(d, J=8.5 Hz, 1H).

### 6.64 Example 64

### N-tert-Butyl-C-(2,4-bis-ethanesulfonylphenyl)nitrone (64)

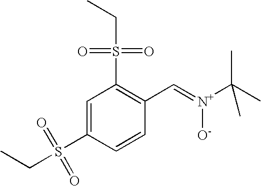

The title compound was prepared according to the procedure described in Example 63, by condensation of N-tert-butylhydroxylamine hydrochloride with 2,4-(bis-ethanesulfanyl) benzaldehyde and subsequent oxidation with oxone. MS: m/z 362 (MH+).

### 6.65 Example 65

### N-tert-Butyl-C-(2,4-bis-2-propanesulfonylphenyl)nitrone (65)

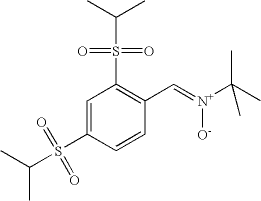

The title compound was prepared according to the procedure described in Example 63, by condensation of N-tert-butylhydroxylamine hydrochloride with 2,4-(bis-2-propanesulfanyl) benzaldehyde and subsequent oxidation with oxone. MS: m/z 390 (MH+).

### 6.66 Example 66

### N-tert-Butyl-C-(2-methanesulfonylphenyl)nitrone (66)

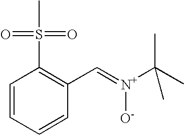

a) 2-Methanesulfanyl benzaldehyde

The title compound was prepared following the procedure described in Example 63a.

b) N-tert-Butyl-C-(2-methanesulfanylphenyl)nitrone

The title compound was prepared following the procedure described in Example 63b.

c) N-tert-Butyl-C-(2-methanesulfonylphenyl)nitrone (66)

Oxone (7.45 g, 12.1 mM; 2.7 eq.) in EDTA (4×10⁻⁴ in 20 ml water,) solution was slowly added during 15 minutes at 0° C. to a suspension of the above sulfanyl nitrone (1.0 g, 4.48 mM; 1.0 eq.) and NaHCO₃ (3.01 g, 35.84 mM; 8.0 eq.) in a mixture of acetone (10 ml) and water (10 ml). The mixture was stirred at the same temperature for an additional 2 hrs before being partitioned between EtOAc and water. The organic layer was separated, washed with water, dried and concentrated. The crude product was chromatographed on silicagel to obtain the title product (900 mg, 87%) as an oil which solidified on long standing. MS: m/z 256 (MH+). ¹H NMR δ 1.53(s, 9H); 3.28(s, 3H); 7.66(dt, J=7.6 Hz, 1.4 Hz, 1H); 7.79 (dt, J=7.6 Hz, 1.4 Hz, 1H); 8.04(dd, J=8.0 Hz, 1.4 Hz, 1H); 8.67(s, 1H); 9.23(dd, J=8.0 Hz, 1.4 Hz, 1H).

### 6.67 Example 67

### N-tert-Butyl-C-(4-ethanesulfonylphenyl)nitrone (67)

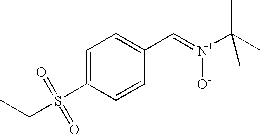

The title compound was prepared according to the procedure described in Example 63, by condensation of N-tert-butylhydroxylamine hydrochloride with 4-(ethanesulfanyl) benzaldehyde and subsequent oxidation with oxone. MS: m/z 270 (MH+).

¹H NMR δ 1.08(t, J=7.4 Hz, 3H); 1.52(s, 9H); 3.29(q, J=7.4 Hz, 2H); 7.90(d, J=8.6 Hz, 2H); 8.07(s, 1H); 8.58(d, J=8.6 Hz, 2H).

### 6.68 Example 68

### N-tert-Butyl-C-(2-ethanesulfonylphenyl)nitrone (68)

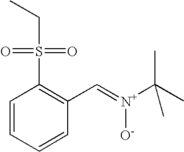

The title compound was prepared according to the procedure described in Example 63, by condensation of N-tert-butylhydroxylamine hydrochloride with 2-(ethanesulfanyl) benzaldehyde and subsequent oxidation with oxone. MS: m/z 270 (MH+).

¹H NMR δ 1.09(t, J=7.2 Hz, 3H); 1.52(s, 9H); 3.38(q, J=7.2 Hz, 2H); 7.66(dt, J=7.8 Hz, 1.4 Hz, 1H); 7.80(dt, J=7.8 Hz, 1.4 Hz, 1H); 8.0(dd, J=7.8 Hz, 1.4 Hz, 1H); 8.58 (s, 1H); 9.25(dd, J=8.0 Hz, 1.4 Hz, 1H).

### 6.69 Example 69

### N-tert-Butyl-C-[2-(2-propanesulfonyl)phenyl]nitrone (69)

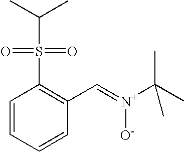

The title compound was prepared according to the procedure described in Example 63, by condensation of N-tert-butylhydroxylamine hydrochloride with 2-(2-propanesulfanyl) benzaldehyde and subsequent oxidation with oxone. MS: m/z 284 (MH+).

¹H NMR δ 1.15(d, J=6.8 Hz, 6H); 1.52(s, 9H); 3.43(quintet, J=6.8 Hz, 1H); 7.66(dt, J=7.8 Hz, 1.4 Hz, 1H); 7.81(dt, J=7.8 Hz, 1.4 Hz, 1H); 7.98(dd, J=7.8 Hz, 1.4 Hz, 1H); 8.59(s, 1H); 9.26(dd, J=8.0 Hz, 1.4 Hz, 1H).

### 6.70 Example 70

### N-tert-Butyl-C-[4-(2-propanesulfonyl)phenyl]nitrone (70)

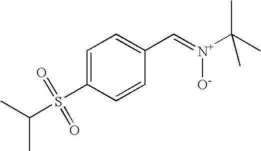

The title compound was prepared according to the procedure described in Example 63, by condensation of N-tert-butylhydroxylamine hydrochloride with 4-(2-propanesulfanyl) benzaldehyde and subsequent oxidation with oxone. MS: m/z 284 (MH+).

¹H NMR δ 1.14(d, 6.8 Hz, 6H); 1.52(s, 9H); 3.41(quintet, 6.8 Hz, 1H); 7.87 (d, 8.6 Hz, 2H); 8.08(s, 1H); 8.58(d, J=8.6 Hz, 2H).

### 6.71 Example 71

### N-tert-Butyl-C-[4-(cyclopentanesulfonyl)phenyl]nitrone (71)

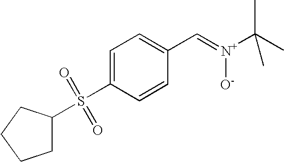

The title compound was prepared according to the procedure described in Example 63, by condensation of N-tert-butylhydroxylamine hydrochloride with 4-(cyclopentanesulfanyl) benzaldehyde and subsequent oxidation with oxone. MS: m/z 310 (MH+).

### 6.72 Example 72

### N-tert-Butyl-C-[2-methanesulfonyl-4-trifluoromethylphenyl]nitrone (72)

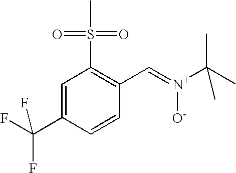

The title compound was prepared according to the procedure described in Example 63, by condensation of N-tert-butylhydroxylamine hydrochloride with 2-methanesulfanyl-4-trifluoromethyl benzaldehyde and subsequent oxidation with oxone. MS: m/z 324 (MH+). ¹H NMR δ 1.55(s, 9H); 3.40(s, 3H); 8.16-8.26(m, 2H); 8.64(s, 1H); 9.43(d, J=8.4 Hz, 1H).

### 6.73 Example 73

### N-tert-Butyl-C-(2-methanesulfonyl-pyridine-3-yl)nitrone (73)

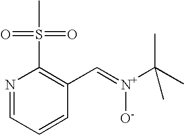

The title compound was prepared according to the procedure described in Example 63, by condensation of N-tert-butylhydroxylamine hydrochloride with 2-methanesulfanyl-pyridine-3-aldehyde and subsequent oxidation with oxone. MS: m/z 257 (MH+). ¹H NMR δ 1.52(s, 9H); 3.46(s, 3H); 7.79(dd, J=8.2 Hz, 4.6 Hz, 1H); 8.65(dd, J=4.6 Hz, 1.6 Hz, 1H); 8.67(s, 1H); 9.65(dd, J=8.2 Hz, 1.6 Hz, 1H).

### 6.74 Example 74

### N-tert-Butyl-C-(2-methanesulfonyl-quinoline-3-yl)nitrone (74)

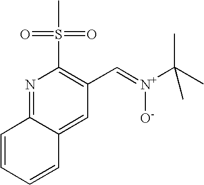

The title compound was prepared according to the procedure described in Example 63, by condensation of N-tert-butylhydroxylamine hydrochloride with 2-methanesulfanyl-quinoline-3-aldehyde and subsequent oxidation with oxone. MS: m/z 307 (MH+). ¹H NMR δ 1.39(s, 9H); 2.34(s, 3H); 7.25-7.33(m, 1H); 7.43(d, J=8.3 Hz, 1H); 7.61-7.67(m, 1H); 7.91(d, 7.8 Hz, 1H); 8.8(s, 1H); 9.80(s, 1H).

### 6.75 Example 75

### N-Benzyl-C-(2-methanesulfonyl-pyridine-3-yl)nitrone (75)

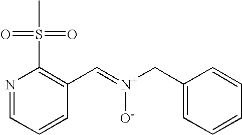

The title compound was prepared according to the procedure described in Example 63, by condensation of N-benzylhydroxylamine hydrochloride with 2-methanesulfanyl-pyridine-3-aldehyde and subsequent oxidation with oxone. MS: m/z 291 (MH+).

### 6.76 Example 76

### N-Cyclohexyl-C-(2-methanesulfonyl-pyridine-3-yl)nitrone (76)

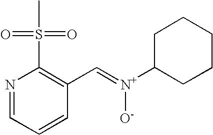

The title compound was prepared according to the procedure described in Example 63, by condensation of N-cyclohexylhydroxylamine hydrochloride with 2-methanesulfanyl-pyridine-3-aldehyde and subsequent oxidation with oxone. MS: m/z 283 (MH+).

### 6.77 Example 77

### N-(tert-Butyl)-C-[2-(methoxycarbonyl)-1H-indol-3-yl]nitrone (77)

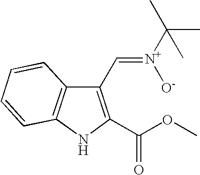

Compound 77 was prepared according to the procedure described in Example 1, starting with N-(tert-butyl)hydroxylamine hydrochloride and 3-formyl-2-(methoxycarbonyl)indole. MS: m/z 275 (MH+).

### 6.78 Example 78

### N-Cyclohexyl-C-[2-(methoxycarbonyl)-1H-indol-3-yl]nitrone (78)

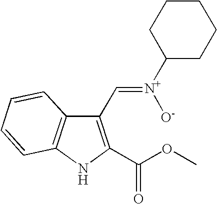

Compound 78 was prepared according to the procedure described in Example 1, starting with N-cyclohexylhydroxylamine hydrochloride and 3-formyl-2-(methoxycarbonyl)indole. MS: m/z 301 (MH+).

### 6.79 Example 79

### N-Benzyl-C-[2-(methoxycarbonyl)-1H-indol-3-yl]nitrone (79)

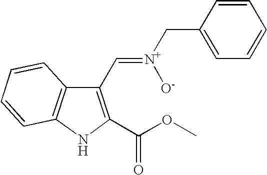

Compound 79 was prepared according to the procedure described in Example 1, starting with N-benzylhydroxylamine hydrochloride and 3-formyl-2-(methoxycarbonyl)indole. MS: m/z 309 (MH+).

### 6.80 Example 80

### N-tert-Butyl-C-(6-methanesulfonyl-pyridine-3-yl)nitrone (80)

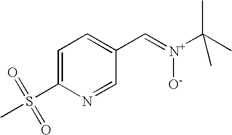

The title compound was prepared according to the procedure described in Example 63, by condensation of N-tert-butylhydroxylamine hydrochloride with 6-methanesulfanyl-pyridine-3-aldehyde and subsequent oxidation with oxone. MS: m/z 257 (MH+). ¹H NMR δ 1.54(s, 9H); 3.28(s, 3H); 8.10(d, J=8.4 Hz, 1H); 8.21(s, 1H); 9.22(dd, J=8.4 Hz, 1.8 Hz, 1H); 9.42(d, J=1.8 Hz, 1H).

### 6.81 Example 81

### N-Benzyl-C-(6-methanesulfonyl-pyridine-3-yl)nitrone (81)

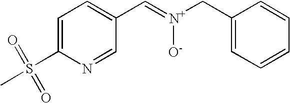

The title compound was prepared according to the procedure described in Example 63, by condensation of N-benzylhydroxylamine hydrochloride with 6-methanesulfanyl-pyridine-3-aldehyde and subsequent oxidation with oxone. MS: m/z 291 (MH+).

### 6.82 Example 82

### Free Radical-Scavenging/Antioxidant Assay of Nitrone Compounds

Nitrones constitute a chemical class of compounds that have antioxidant properties due to their ability to form stable adducts (i.e., spin traps) with free radicals (See, e.g., Janzen, E. G. et al., 1992, Stabilities of Hydroxyl Radical Spin Adducts of PBN-Type Spin Traps, Free Radical Biol. Med., 12(2): 169-73). Because free radicals can cause oxidative damage to cellular constituents (e.g., proteins and lipids), which can lead to pathological consequences, it has been reported that the antioxidant properties of nitrone compounds at least partly underlie their therapeutic potential, as reported in studies using a canonical member of this chemical class, C-(phenyl)-N-(tert-butyl)nitrone (PBN) (See, e.g., J. M. Carney and R. A. Floyd, 1991, Protection against Oxidative Damage to CNS by α-Phenyl-tert-butylnitrone (PBN) and Other Spin-Trapping Agents: a Novel Series of Nonlipid Free Radical Scavengers, J. Mol. Neurosci., 3(1): 47-57, and Thomas, C. E. et al., 1994, Multiple Mechanisms for Inhibition of Low Density Lipoprotein Oxidation by Novel Cyclic Nitrone Spin Traps, J. Biol. Chem., 269(45): 28055-61).

Therefore, nitrone compounds that have improved antioxidant activity compared to PBN can have better therapeutic potential than PBN. More generally, diseases or conditions that have been reported to be susceptible to antioxidant therapy or that involve the generation of free radicals may be susceptible to nitrone treatment based on the antioxidant activity of nitrones. Diseases or conditions that arise from or are characterized by oxidative damage or oxidative stress include, but are not limited to, neurodegenerative, autoimmune and inflammatory diseases or conditions.

Nitrone compounds of the present invention were tested for their free-radical scavenging/antioxidant activity in an in vitro assay that is accepted by those skilled in the art as a model for conditions involving the generation of free radicals. The assay is based on a reaction between a free-radical donor, 2,2-diphenyl-1-picrylhydrazyl (DPPH), and a radical scavenger/antioxidant to be tested for free-radical scavenging activity. Upon donation of the free-radical electron to the purported radical scavenger, the peak visible absorbance of DPPH (515-520 nm) decreases so that optical density readings at this part of the visual spectrum reflect the progression of the following reaction:
DPPH•+AH→DPPH-H+A•
where AH is a hypothetical radical scavenger/antioxidant. The assay is based on a protocol originally detailed in Brand-Williams, W. et al., 1995, Use of a Free Radical Method to Evaluate Antioxidant Activity, Lebensm. Wiss. Technol., 28:25-30, with further modifications described in L. R. Fukumoto and G. Mazza, 2000, Assessing Antioxidant and Prooxidant Activities of Phenolic Compounds, J. Agric. Food Chem., 48:3597-3604.

The antioxidant assay was performed using Perkin-Elmer 96-well, clear-bottom, black-wall plates (ordered from E & K Scientific Products) and a Tecan Safire absorbance plate reader. The positive controls were Trolox (6-hydroxy-2,5,7,8-tetramethylchromane-2-carboxylic acid, Sigma-Aldrich), BHA (2(3)-tert-butylhydroquinone monomethyl ether, Sigma-Aldrich), PBN (C-(phenyl)-N-(tert-butyl)nitrone, Sigma-Aldrich) and S-PBN (C-(2-sulfophenyl)-N-(tert-butyl)nitrone, sodium salt, prepared according to E. G. Janzen and R. V. Shetty, 1979, Tetrahedron Lett., 35: 3229-32), and the negative control (i.e., vehicle) was DMSO. In brief, 2 μL of 100× DMSO stock of the desired final concentration of each control or nitrone compound to be tested in the same batch was added to a separate well. To each well was then added 198 μL of a freshly made 50 μM DPPH (Sigma-Aldrich) solution in 80% methanol using a multi-channel pipette. The absorbance was immediately read on the plate reader at 520 nm and thereafter read periodically to assess kinetics until all reactions reached completion (i.e., steady state). Since the steady-state point was 24 h, the assay results are shown from the 24 h time point. The absorbance at 520 nm (OD) was plotted versus the concentrations of the controls and nitrone compounds to assess dose-response and interpolate the EC₅₀ values of the controls and test compounds.

In this antioxidant assay, exemplary compounds of the invention exhibited EC₅₀ values as shown in Table 1.

TABLE 1
DPPH Assay Data

| Compound | MW | EC50 (μM) |
|---|---|---|
| BHA | | +++++ |
| PBN | | + |
| SPBN | | + |
| Trolox | | +++++ |
| 1 | 235.28 | ++ |
| 2 | 261.32 | + |
| 3 | 269.30 | ++ |
| 4 | 295.33 | +++ |
| 5 | 321.37 | +++ |
| 6 | 329.35 | +++ |
| 7 | 221.25 | +++ |
| 8 | 247.29 | ++++ |
| 9 | 255.27 | ++++ |
| 10 | 281.31 | ++ |
| 11 | 307.34 | ++++ |
| 12 | 315.32 | ++++ |
| 16 | 248.32 | +++ |
| 20 | 256.32 | + |
| 22 | 339.46 | + |
| 23 | 326.41 | + |
| 24 | 284.38 | + |
| 25 | 350.41 | + |
| 26 | 333.41 | + |
| 28 | 324.44 | + |
| 29 | 310.42 | + |
| 30 | 312.43 | + |
| 31 | 338.47 | + |
| 32 | 346.45 | + |
| 33 | 324.44 | + |
| 34 | 350.48 | + |
| 35 | 358.46 | + |
| 36 | 352.45 | + |
| 37 | 360.43 | +++ |
| 38 | 339.46 | + |
| 39 | 365.50 | + |
| 40 | 373.47 | + |
| 41 | 346.45 | + |
| 42 | 372.49 | + |
| 43 | 380.47 | + |
| 44 | 372.49 | + |
| 45 | 398.52 | + |
| 46 | 406.50 | + |
| 47 | 270.35 | + |
| 48 | 312.43 | + |
| 49 | 346.45 | + |
| 50 | 312.43 | + |
| 51 | 298.40 | + |
| 54 | 346.45 | + |
| 56 | 372.49 | + |
| 57 | 380.47 | + |
| 58 | 312.43 | + |
| 59 | 298.40 | + |
| 60 | 338.47 | + |
| 61 | 346.45 | + |
| 62 | 255.34 | + |
| 63 | 333.43 | ++ |
| 64 | 361.48 | ++ |
| 65 | 389.53 | ++ |
| 66 | 255.34 | ++ |
| 67 | 269.36 | + |
| 68 | 269.36 | ++ |
| 69 | 283.39 | + |
| 70 | 283.39 | + |
| 71 | 309.43 | + |
| 72 | 323.33 | + |
| 73 | 256.32 | + |
| 74 | 306.38 | + |
| 75 | 290.34 | + |
| 76 | 282.36 | + |
| 77 | 274.32 | + |
| 78 | 314.38 | + |
| 79 | 308.34 | + |

*EC₅₀ is the concentration at which a compound reduces by 50% the peak absorbance of DPPH at 520 nm.
+++++ EC₅₀ < 10 μM
++++ 100 μM > EC₅₀ > 10 μM
+++ 500 μM > EC₅₀ > 100 μM
++ 1000 μM > EC₅₀ > 500 μM
+ EC₅₀ > 1000 μM

As can be seen from Table 1, nitrone compounds of the present invention possess significant or potent free-radical scavenging/antioxidant activity. Indeed, many of the nitrone compounds of the invention display comparable or even greater antioxidant activity than PBN. Accordingly, the aryl, heteroaromatic and bicyclic aryl nitrone compounds of the invention are potential therapeutic agents useful for the treatment and/or prevention of diseases or conditions that have been reported to be amenable to antioxidant therapy or involve free-radical generation. Such diseases or conditions include, but are not limited to, pain conditions, autoimmune diseases or conditions, inflammatory diseases or conditions, and neurological or neurodegenerative diseases or conditions.

Non-limiting examples of pain conditions that arise from or are characterized by oxidative damage or oxidative stress are: migraine (See, e.g., Ciancarelli, I. et al., 2003, Urinary Nitric Oxide Metabolites and Lipid Peroxidation By-Products in Migraine, Cephalalgia, 23(1): 39-42); acute, chronic and neuropathic pain syndromes and neuralgias (See, e.g., De las Heras Castano, G. et al., 2000, Use of Antioxidants to Treat Pain in Chronic Pancreatitis, Rev. Esp. Enferm. Dig., 92(6): 375-85);irritable bowel syndrome; and nerve injury and neuropathies including diabetic neuropathy (See, e.g., Gray, C. et al., 2003, Neuroprotective Effects of Nitrone Radical Scavenger S-PBN on Reperfusion Nerve Injury in Rats, Brain Res., 982(2): 179-85, and Strokov, I. A. et al., 2000, The Function of Endogenous Protective Systems in Patients with Insulin-Dependent Diabetes Mellitus and Polyneuropathy: Effect of Antioxidant Therapy, Bull. Exp. Biol. Med., 130(10): 986-90). Non-limiting examples of autoimmune diseases or conditions that arise from or are characterized by oxidative damage or oxidative stress are: multiple sclerosis (See, e.g., Liu, Y. et al., 2003, Bilirubin as a Potent Antioxidant Suppresses Experimental Autoimmune Encephalomyelitis: Implications for the Role of Oxidative Stress in the Development of Multiple Sclerosis, J. Neuroimmunol., 139(1-2): 27-35); arthritis; diabetes and related complications (See, e.g., Tabatabaie, T. et al., 1997, Spin Trapping Agent Phenyl-N-tert-butylnitrone Protects against the Onset of Drug-Induced Insulin-Dependent Diabetes Mellitus, FEBS Lett., 407(2): 148-52); and Graves' disease and other thyroid disorders (See, e.g., Vrca, V. B. et al., 2004, Supplementation with Antioxidants in the Treatment of Graves' Disease: the Effect on Glutathione Peroxidase Activity and Concentration of Selenium, Clin. Chim. Acta., 341(1-2): 55-63).

Non-limiting examples of inflammatory diseases or conditions that arise from or are characterized by oxidative damage or oxidative stress are: myocardial infarction and dysfunction (See, e.g., Vergely, C. et al., 2003, Effect of Two New PBN-Derived Phosphorylated Nitrones against Postischaemic Ventricular Dysrhythmias, Fundam. Clin. Pharmacol., 17(4): 433-42); arteriosclerosis and other vascular diseases (See, e.g., Micheletta, F. et al., 2004, Vitamin E Supplementation in Patients with Carotid Atherosclerosis: Reversal of Altered Oxidative Stress Status in Plasma But Not in Plaque, Arterioscler. Thromb. Vasc. Biol., 24(1): 136-40); asthma, reactive airway diseases and allergies (See, e.g., Nadeem, A. et al., 2003, Increased Oxidative Stress and Altered Levels of Antioxidants in Asthma, J. Allergy Clin. Immunol., 111(1): 72-8); transplant and graft failure or rejection (See, e.g., Connor, H. D. et al., 1992, Evidence that Free Radicals Are Involved in Graft Failure following Orthotopic Liver Transplantation in the Rat—an Electron Paramagnetic Resonance Spin Trapping Study, Transplantation, 54(2): 199-204); lung injury and damage (See, e.g., Murphy, P. G. et al., 1991, Direct Detection of Free Radical Generation in an in vivo Model of Acute Lung Injury, Radical Res.Commun., 15(3): 167-76); hepatitis and jaundice-induced liver disorders (See, e.g., Yamashita, T. et al., 1996, The Effects of α-Phenyl-tert-butylnitrone (PBN) on Copper-Induced Rat Fulminant Hepatitis with Jaundice, Free Radical Biol. Med., 21(6): 755-61); pancreatitis and other pancreatic disorders (See, e.g., Koiwai, T. et al., 1989, The Role of Oxygen Free Radicals in Experimental Acute Pancreatitis in the Rat, Int. J. Pancreatol., 5(2): 135-43); inflammatory bowel disease including Crohn's disease and other disorders of the digestive tract (See, e.g., Reimund, J. M. et al., 1998, Antioxidants Inhibit the in vitro Production of Inflammatory Cytokines in Crohn's Disease and Ulcerative Colitis, Eur. J. Clin. Invest., 28(2): 145-50); retinal ischemia and damage including macular degeneration and other degenerative or inflammatory disorders of the retina and eye (See, e.g., F. Block and M. Schwarz, 1997, Effects of Antioxidants on Ischemic Retinal Dysfunction, Exp. Eye Res., 64(4): 559-64); renal ischemia and kidney disorders (See, e.g., Kadkhodaee, M. et al., 1996, Detection of Hydroxyl and Carbon-Centered Radicals by EPR Spectroscopy after Ischaemia and Reperfusion of the Rat Kidney, Free Radical Res., 25(1): 31-42); and endotoxemia (See, e.g., Harkins, J. D. et al., 1997, Effect of α-Phenyl-tert-butylnitrone on Endotoxin Toxemia in Horses, Vet. Hum. Toxicol., 39(5): 268-71).

Non-limiting examples of neurological or neurodegenerative diseases or conditions that arise from or are characterized by oxidative damage or oxidative stress are: stroke (See, e.g., Marshall, J. W. et al., 2001, NXY-059, a Free Radical-Trapping Agent, Substantially Lessens the Functional Disability Resulting from Cerebral Ischemia in a Primate Species, Stroke, 32(1): 190-98, and Ginsberg, M. D. et al., 2003, Stilbazulenyl Nitrone, a Novel Antioxidant, Is Highly Neuroprotective in Focal Ischemia, Ann. Neurol., 54(3): 330-42); schizophrenia and other disorders of cognition (See, e.g., Dakhale, G. et al, 2004, Oxidative Damage and Schizophrenia: the Potential Benefit by Atypical Antipsychotics, Neuropsychobiol., 49(4): 205-09); mood disorders and other disorders of affect (See, e.g., Ranjekar, P. K. et al., 2003, Decreased Antioxidant Enzymes and Membrane Essential Polyunsaturated Fatty Acids in Schizophrenic and Bipolar Mood Disorder Patients, Psychiatry Res., 121(2): 109-22); epilepsy (See, e.g., Gupta, M. et al., 2004, Add-on Melatonin Improves Quality of Life in Epileptic Children on Valproate Monotherapy: a Randomized, Double-Blind, Placebo-Controlled Trial, Epilepsy Behav., 5(3): 316-21); aging and senescence (See, e.g., Carney, J. M. et al., 1991, Reversal of Age-Related Increase in Brain Protein Oxidation, Decrease in Enzyme Activity, and Loss in Temporal and Spatial Memory by Chronic Administration of the Spin-Trapping Compound N-tert-Butyl-α-phenylnitrone, Proc. Natl. Acad. Sci. USA, 88(9): 3633-6); Parkinson's disease (See, e.g., Fredriksson, A. et al., 1997, MPTP-Induced Deficits in Motor Activity: Neuroprotective Effects of the Spin-Trapping Agent, α-Phenyl-tert-butylnitrone (PBN), J. Neural. Transm., 104(6-7): 579-92); Alzheimer's disease (See, e.g., Butterfield, D. A. et al., 1996, A β(25-35) Peptide Displays H₂O₂-Like Reactivity towards Aqueous Fe²⁺, Nitroxide Spin Probes, and Synaptosomal Membrane Proteins, Life Sci., 58(3): 217-28); Huntington's disease (See, e.g., Nakao, N. et al., 1996, Antioxidant Treatment Protects Striatal Neurons against Excitotoxic Insults, Neuroscience, 73(1): 185-200); amyotrophic lateral sclerosis (See, e.g., Desnuelle, C. et al., 2001, A Double-Blind, Placebo-Controlled Randomized Clinical Trial of α-Tocopherol (Vitamin E) in the Treatment of Amyotrophic Lateral Sclerosis, Amyotrophic Lateral Scler. Other Motor Neuron Disorders, 2(1): 9-18); and head trauma and traumatic brain injury (See, e.g., Sen, S. et al., 1994, α-Phenyl-tert-butylnitrone Inhibits Free Radical Release in Brain Concussion, Free Radical Biol. Med., 16(6): 685-91, and Marklund, N. et al., 2001, Effects of the Nitrone Radical Scavengers PBN and S-PBN on in vivo Trapping of Reactive Oxygen Species after Traumatic Brain Injury in Rats, J. Cereb. Blood Flow Metab., 21(11); 1259-67).

### 6.83 Example 83

### Pharmacokinetic Evaluation of Aryl Nitrone Compounds of the Invention following Intravenous and Oral Administration in Rats

Male Sprague-Dawley rats were given at least 24 hours acclimation before experiment initiation. During acclimation period, all animals received food and water ad libitum. However, food (not water) was removed from the animal's cages up to 12 hours before initiation of the experiment. During the first 4 hours of experimentation, the animals received only water ad libitum. Two to three animals for iv and three animals for oral administration were tested. For iv formulation nitrone compounds of this invention were dissolved (1 mg/mL) in a mixture of 5% dimethyl acetamide (v/v), 0 to 4% Tween 80 (v/v), 10 to 40% PEG 400 (v/v) and the rest percentage of water (v/v). For oral formulation nitrone compounds of this invention were dissolved (2 mg/mL) in a mixture of 4% of 10% Tween in water and 96% of 0.5% carboxymethyl cellulose (medium viscosity) in water; or 4% of 10% Tween in water, 48% of 0.5% carboxymethyl cellulose (medium viscosity) in water, and 48% of 0.5% Hydroxypropyl Methylcellulose/0.2% Sodium Lauryl Sulfate in water. These formulations were stored at 5° C. until the experiment. Formulations were then stir-mixed at least half an hour before dosing. Exactly 200 μL of each left-over formulation was diluted with CH₃CN/H₂O for concentration analysis. The animals were weighed before dosing. The body weight was used to calculate the true dose for each animal:

IV dosing:
Dose volume (mL)=1.0 mL/kg
The intravenous dose was administered through the jugular vein catheter or tail vein in less than 1 minute.

PO dosing:
Dose volume (mL)=2.5 mL/kg
The oral dose was administered by oral gavage.

For IV dosing, blood samples were collected (using a pre-heparinized syringe) via the carotid artery orjugular vein catheter at t=2, 5, 15, 30, 60, 120, 180, 360, and 480 minutes post dosing. For PO dosing, blood samples were collected (using a pre-heparinized syringe) via the carotid artery or jugular vein catheter before dosing and at t=5, 15, 30, 60, 120, 180, 360, and 480 minutes post dosing. For some nitrone compounds a 1440 minutes (24 hours) sample was also taken for both IV and PO administrations. About 250 μL of blood was obtained at each time point from the animals. Equal volumes of 0.9% normal saline were replaced to prevent dehydration. The whole blood samples were maintained on ice until centrifugation. Blood samples were then centrifuged at 14,000 rpm for 10 minutes at 4° C. and the upper plasma layer transferred into a clean vial and stored at −80° C. The resulting plasma samples were then analyzed by mass spectroscopy using standard methods.

### 6.84 Example 84

### LC/MS/MS Method for the Analysis of Aryl Nitrone Compounds of the Invention in Rat Plasma

All the samples from above assays were analyzed on a PE-Sciex API 3000 triple adrupole with a Turbo Ion Spray source. Nitrone compounds of this invention were seperated from the matrix via a linear gradient reverse-phase chromatography using a C18 column, such as Thermo BDS Hypersil C₁₈ (100×4.6 mm, 5 micron particle, 120 Å pore size). The mobile Phases were:
A: 200 mL CH₃CN, 1800 mL H₂O, 1.54 g NH₄OAc, and 2 mL formic acid
B: 1800 mL CH₃CN, 200 mL H₂O, 1.54 g NH₄OAc, and 2 mL formic acid
   Nitrone compounds were detected by the mass spectrometer in the positive ion multiple reaction monitoring mode (MRM). For quantitative analysis, a standard curve was prepared by spiking a stock solution of the nitrone compound to the appropriate matrix to achieve a quantitation curve range and analyzed the standards in the same manner as the samples.

Pharmacokinetic parameters of the aryl nitrone compounds were determined by a noncompartmental analysis using WinNonlin-Pro (Version 4.1, Pharsight Corporation). Average and standard deviation of the parameters were calculated using standard formulas in Microsoft Excel. Pharmakokinetic parameters are presented in Table 2.

TABLE 2
Pharmacokinetic Data for Nitrone Compounds

| | | | | | | Cₘₐₓ | Tₘₐₓ |
|---|---|---|---|---|---|---|---|
| | | | T_{1/2} | CI | Vd | (PO) | (PO) |
| Compd | MW | F (%) | (hr) | (L/h/kg) | (L/Kg) | (ng/mL) | (hr) |
| 13 | 221.26 | 64.7 | 0.37 | 0.77 | 0.41 | 5580 | 0.33 |
| 18 | 276.38 | 75.1 | 0.33 | 1.29 | 0.62 | 2237 | 0.42 |
| 19 | 220.27 | 39.5 | 3.64 | 0.14 | 0.74 | 2997 | 0.33 |
| 20 | 256.32 | 55.4 | 3.67 | 0.77 | 4.08 | 796 | 0.37 |
| 22 | 339.46 | 15.6 | 0.57 | 2.21 | 1.83 | 703 | 0.24 |
| 23 | 326.41 | 65.3 | 1.58 | 1.68 | 3.78 | 1125 | 0.42 |
| 24 | 284.38 | 7.19 | 0.52 | 3.54 | 2.58 | 198 | 0.20 |
| 25 | 350.41 | 0.85 | 0.43 | 1.76 | 1.12 | 10 | 0.25 |
| 26 | 333.41 | 43.4 | 4.37 | 0.01 | 0.09 | 27333 | 0.42 |
| 27 | 326.41 | 18.1 | 0.38 | 4.73 | 2.62 | 423 | 0.084 |
| 28 | 324.44 | 13.8 | 13.03 | 1.14 | 19.85 | 134 | 1.08 |
| 29 | 310.42 | 4.3 | 0.29 | 2.28 | 0.97 | 114 | 0.19 |
| 30 | 312.43 | 1.7 | 0.51 | 5.63 | 4.15 | 40 | 0.083 |
| 33 | 324.44 | 0.1 | 0.54 | 2.17 | 1.66 | 10 | 0.083 |
| 48 | 312.43 | 55.3 | 0.49 | 1.97 | 1.39 | 862 | 0.25 |
| 54 | 346.45 | 13.2 | 1.03 | 2.15 | 3.21 | 180 | 0.25 |
| 60 | 338.47 | 0.4 | 0.51 | 3.8 | 2.84 | 1.59 | 0.14 |
| 62 | 255.34 | 63.4 | 5.82 | 0.06 | 0.53 | 4633 | 0.41 |
| 63 | 333.43 | 95.9 | 1.66 | 0.19 | 0.45 | 4130 | 0.75 |
| 64 | 361.48 | 91.3 | 0.55 | 0.83 | 0.67 | 2493 | 0.66 |
| 65 | 389.53 | 13.7 | 0.35 | 1.89 | 0.98 | 339 | 0.24 |
| 66 | 255.34 | 70.3 | 2.39 | 0.18 | 0.63 | 4733 | 1.00 |
| 67 | 269.36 | 46.4 | 4.43 | 0.07 | 0.42 | 4763 | 1.33 |
| 68 | 269.36 | 36.4 | 0.93 | 0.47 | 0.63 | 2107 | 0.99 |
| 69 | 283.39 | 54.9 | 0.64 | 0.84 | 0.76 | 1837 | 0.19 |
| 70 | 283.39 | 52.2 | 0.62 | 1.2 | 1.07 | 1273 | 0.49 |
| 71 | 309.43 | 5.87 | 0.76 | 1.86 | 1.66 | 108 | 0.28 |
| 72 | 323.33 | 59.8 | 2.82 | 0.66 | 2.69 | 1523 | 0.32 |
| 73 | 256.32 | 72.2 | 2.76 | 0.2 | 0.8 | 4167 | 1.42 |
| 74 | 306.38 | 9.4 | 0.32 | 22.57 | 10.68 | 12 | 0.19 |
| 75 | 290.34 | 50.2 | 0.57 | 1.31 | 1.07 | 1031 | 0.19 |
| 80 | 256.33 | 54.7 | 1.71 | 0.21 | 0.52 | 3943 | 0.5 |

MW: Molecular weight of the nitrone compound.
F (%): Oral bioavailability, calculated by dividing the plasma exposure of oral dose with that of the intravenous dose, normalized to their respective doses.
T_{1/2}: Elimination half life of the nitrone compound.
Cl: Clearance of the nitrone compound obtained from intravenous administration.
Vd: Volume of distribution of the nitrone compound obtained from intravenous administration.
Cₘₐₓ: Maximal plasma concentration of the nitrone compound detected following oral administration.
Tₘₐₓ: Time taken to reach maximal plasma concentration of the nitrone compound following oral administration.

The aryl nitrone compounds of this invention have favorable pharmacokinetic properties. Most compounds displayed low to moderate clearance. While a range of volume of distribution (from low to high) was observed, more than half the compounds displayed volume of distribution greater than rat body water volume, suggesting tissue distribution. When administered orally, the nitrone compounds were absorbed rapidly, as demonstrated by the short Tₘₐₓ (<0.5 hr for majority of the compounds). Oral exposure was generally high and more than 60% of the compounds displayed oral bioavailability >30%.

### 6.85 Example 85

### Plasma Protein Binding of Aryl Nitrone Compounds of the Invention

Nitrone compounds of this invention were individually dissolved in DMSO to make a stock solution of 1 mg/mL. The compound was spiked into plasma to achieve a final concentration of 1 μg/mL. Spiked plasma and phosphate buffer (0.1M, pH 7.4), 200 μl each, were added to the opposite sides of the membrane in a 96-well equilibrium dialyzer. The dialyzer plate was then covered and equilibrated overnight at 37° C. on an orbital shaker. Aliquots were taken from the plasma and the buffer compartments and prepared by adding blank plasma to samples from the buffer compartments and drug-free phosphate buffer to samples from the plasma compartments to eliminate the matrix effects. The samples were extracted using protein precipitation procedure by adding CH₃CN. The samples were analyzed using a LC/MS/MS method. The percentage of free and bound nitrone compound were calculated according to the following formula:
% Free=[Free Drug/Total Drug]*100=[(Peak Area)_{buffer}/(Peak Area)ₛₑᵣᵤₘ]*100
% Bound=100−% Free

TABLE 3
Plasma Protein Binding of Nitrone Compounds

Plasma Protein Binding

| Compound | MW | % Bound Rat | % Bound Human |
|---|---|---|---|
| 20 | 256.32 | 2.41 | 0 |
| 23 | 326.41 | 21.8 | 20 |
| 26 | 333.41 | 99 | 76.2 |
| 62 | 255.34 | 0.06 | 17.4 |
| 63 | 333.43 | 25.9 | 13.3 |
| 64 | 361.48 | 18.9 | 30.3 |
| 66 | 255.34 | 10.2 | 27.4 |
| 67 | 269.36 | 22 | 13.6 |
| 68 | 269.36 | 21.7 | 21.9 |
| 69 | 283.39 | 24.5 | 46.8 |
| 70 | 283.39 | 28.2 | 20.4 |
| 71 | 309.43 | 51.6 | 45.6 |
| 72 | 323.33 | 68.1 | 63.3 |

The aryl nitrone compounds of this invention displayed low plasma-protein binding. Most of the compounds (10 out of 13) had less than 30% binding values. Consequently the aryl nitrone compounds have the potential to reach their in vivo targets and to exert their pharmacological effects.

### 6.86 Example 86

### Brain Penetration of Aryl Nitrone Compounds of the Invention

Nitrone compounds of this invention were formulated individually as suspensions and administered as a single dose to Sprague-Dawley rats via oral gavage (compound 26 at 5 mg/kg, compound 62 at 15 mg/kg, compounds 20, 63 and 66 at 50 mg/kg). Plasma samples were obtained at or near Tₘₐₓ projected at the given dose for each compound and the animals were euthanized using carbon dioxide. Immediately following euthanization, cerebrospinal fluid (CSF) was obtained by cisternal puncture of the atlanto-occipital membrane and drawn from the magnum cisternum. The brain was first perfused intracardially with ˜150 mL of ice-cold 0.1 M Phosphate Buffered Saline (PBS) at pH7.4. Following the removal of the dura, the brain was weighed. The brain was then dissected into smaller pieces and rinsed twice with ˜10 mL PBS. The brain, CSF, and plasma samples were frozen on dry ice and stored at −80° C. before analysis. CSF and plasma samples were subjected to a protein precipitation method prior to LC/MS/MS analysis. Blank rat plasma CSF were used accordingly for diluting the samples when needed. Bioanalytical standard curves were prepared by spiking a stock solution of the nitrone compound to blank rat plasma or CSF to achieve a quantitation curve range and analyzed the standards in the same manner as the samples. Brain samples underwent homogenization in 2 mL water and liquid-liquid extraction with ethyl acetate three times. The combined organic phase for each sample was evaporated under a stream of nitrogen at 40° C. and the residues were reconstituted with an appropriate amount of mobile phase B (referring to LC/MS/MS method section). A bioanalytical standard curve for brain analysis was prepared by spiking 100 μL of stock solution directly into sliced blank rat brain purchased from Pelfreeze. The piked brains then underwent the same processing procedures for the dosed samples.

The reconstituted samples were vortexed and incubated to fully dissolve the analytes. The samples were centrifuged, and then further diluted with Mobile phase B if necessary before LC/MS/MS analysis. Nitrone compound levels in the brain were calculated based on the measured concentration, the volume of reconstitution and brain weight to yield a unit of ng (of compound) per g of brain. To calculate the brain/plasma ratio (w/v), it was assumed that 1 g of brain tissue takes approximately 1 mL of volume.

As shown in Table 4, a majority of the nitrone compounds had good brain penetration properties with 3 out 5 compounds having a brain/plasma ratio >20%.

TABLE 4
Brain Penetration of Nitrone Compounds

Brain Penetration (Rat)

| | | | Brain/Plasma |
|---|---|---|---|
| Compound | MW | CSF/Plasma (%) | (%) |
| 20 | 256.32 | 14.4 | 22.9 |
| 26 | 333.41 | 0.12 | 0.56 |
| 62 | 255.34 | 78.6 | 62.5 |
| 63 | 333.43 | 33.6 | 18.9 |
| 66 | 255.34 | 93.6 | 22.4 |

### 6.87 Example 87

### Solubility Measurements of Nitrone Compounds at pH 7.4

Nitrone compounds (>3 mg) of this invention were mixed with a phosphate buffer at pH 7.4 to make a >0.3 mg/mL mixture. The mixture was vortexed for more than 2 hours and equilibrated over 12 hours at room temperature. The equilibrated mixture was used to saturate a 0.45μm Tuffryn syringe filter. After saturating, the remainder of the mixture was filtered through the saturated filter. The filtrate was diluted by 1, 10, 100, and 1000 fold and analyzed using a LC/MS/MS method with standard curve ranging from 1 to 1000 ng/mL.

TABLE 5
Solubility of Nitrone Compounds

| | | Solubility @ pH 7.4 | Solubility @ pH |
|---|---|---|---|
| Compound | MW | (μg/mL) | 7.4 (μM) |
| 13 | 221.26 | 2000 | 9039 |
| 18 | 276.38 | 376 | 1360 |
| 19 | 220.27 | 3130 | 14210 |
| 30 | 312.43 | 791 | 2532 |
| 31 | 338.47 | 978 | 2889 |
| 33 | 324.44 | >1080 | >3329 |
| 34 | 350.48 | 500 | 1425 |
| 35 | 358.46 | 38.4 | 107 |
| 36 | 352.45 | 206 | 584 |
| 37 | 360.43 | 1810 | 5022 |
| 38 | 339.46 | >632 | >1862 |
| 39 | 365.5 | >962 | >2632 |
| 40 | 373.47 | >851 | >2279 |
| 41 | 346.45 | 143 | 413 |
| 42 | 372.49 | 25.8 | 69.2 |
| 43 | 380.47 | 66 | 173 |
| 44 | 372.49 | 58.1 | 156 |
| 45 | 398.52 | 1.42 | 3.5 |
| 46 | 406.5 | 2.14 | 5.26 |
| 73 | 256.32 | >2900 | >11314 |
| 74 | 306.38 | 29.4 | 96 |
| 75 | 290.34 | 848 | 2921 |
| 76 | 282.36 | >1150 | >4072 |

As shown in Table 5, the aryl nitrone compounds of this invention displayed high aqueous solubility at pH 7.4. 38 of the 42 compounds tested had solubility greater than 10 μg/mL. 26 Compounds had solubility greater than 100 μg/mL, and 6 compounds had more than 1 mg/mL solubility. The favorable aqueous solubility contributes to the high oral bioavailability of these compounds.

### 6.88 Example 88

### Microsomal Stability of Aryl Nitrone Compounds of the Invention

Frozen Sprague-Dawley rat liver microsomes (RLM) were thawed on ice and gently mixed before use. The final reaction mixture consisted of a nitrone compound of this invention (at ˜500 ng/ml), 1 mM NADPH, and 0.5 mg/ml of RLM protein in 0.1 M PBS (pH7.4), with organic solvent concentration not exceeding 1% (v/v). Per set of incubations, a positive control compound was included. The mixture was first pre-incubated 3 to 5 minutes at 37° C. without NADPH, and the reaction was then initialized by the addition of NADPH and incubated at 37° C. for up to 30 minutes. An aliquot of the reaction mixture was sampled at the initiation of the reaction and at designated time after reaction started. The drawn samples were quenched with acetonitrile, diluted with mobile phase to ensure the detection of test article in the linear range, and analyzed by LC/MS/MS. Half-life or percentage of remaining nitrone compound was calculated using standard methods. A similar method, or a slight variation of it, was used to test the stability of nitrones in human liver microsomes (HLM).

TABLE 6
Stability of Nitrone Compounds

| | | % of Nitrone | % of Nitrone |
|---|---|---|---|
| | | Remaining at 30 min | Remaining at 30 min |
| Compd | MW | (Human) | (Rat) |
| 13 | 221.26 | NT | 100 |
| 18 | 276.38 | NT | 100 |
| 19 | 220.27 | NT | 100 |
| 30 | 312.43 | NT | 7.4 |
| 31 | 338.47 | NT | 0 |
| 32 | 346.45 | NT | 0 |
| 33 | 324.44 | NT | 0.3 |
| 34 | 350.48 | NT | 0 |
| 35 | 358.46 | NT | 0 |
| 36 | 352.45 | 24.2 | NT |
| 37 | 360.43 | 65.4 | NT |
| 38 | 339.46 | 76.6 | NT |
| 39 | 365.50 | 41.9 | NT |
| 40 | 373.47 | 27.9 | NT |
| 41 | 346.45 | 0 | NT |
| 42 | 372.49 | 0 | NT |
| 43 | 380.47 | 0 | NT |
| 44 | 372.49 | 0 | NT |
| 45 | 398.52 | 0 | NT |
| 46 | 406.50 | 0 | NT |
| 47 | 270.35 | 100 | NT |
| 48 | 312.43 | 100 | NT |
| 49 | 346.45 | 0 | NT |
| 50 | 312.43 | 86 | NT |
| 51 | 298.40 | 94 | NT |
| 52 | 338.47 | 64 | NT |
| 53 | 346.45 | 65 | NT |
| 54 | 346.45 | 62 | NT |
| 55 | 332.42 | 82 | NT |
| 56 | 372.49 | 33 | NT |
| 57 | 380.47 | 14 | NT |
| 58 | 312.43 | 100 | NT |
| 59 | 298.40 | 100 | NT |
| 60 | 338.47 | 100 | NT |
| 61 | 346.45 | 95 | NT |
| 62 | 255.34 | 100 | 100 |
| 63 | 333.43 | 99 | 97 |
| 64 | 361.48 | 100 | 100 |
| 65 | 389.53 | 94 | 78 |
| 73 | 256.32 | 100 | NT |
| 74 | 306.38 | 75.7 | NT |
| 75 | 290.34 | 95.1 | NT |
| 76 | 282.36 | 90.5 | NT |
| 80 | 256.33 | 94.5 | NT |
| 81 | 290.34 | 90.5 | NT |

The nitrone compounds of this invention are generally stable in human or rat liver microsomes. Among the 45 compounds tested, 23 compounds displayed more than 75% compound remaining after a 30 minutes of incubation with either rat or human liver microsomes with the addition of NADPH. The high stability indicated a slow rate of oxidative metabolism of these compounds by the liver, which in turn resulted in a low clearance and a high oral bioavailability. The microsomal stability data are consistent with the pharmacokinetic results.

### 6.89 Example 89

### Compound 62 is Effective in Vivo Against Diabetic Neuropathy (Mechanical Hyperalgesia)

In this example, the ability of Compound 62 to produce beneficial effects in protecting against and/or reversing the pathology of neuropathy in a streptozotocin (STZ)-induced rat model of diabetes. To evaluate if chronic treatment with Compound 62 protects the diabetic animals from developing neuropathy, they were examined for mechanical hyperalgesia responses.

Adult male Sprague Dawley (SD) rats weighing 250-300 gm (Charles River Laboratories, San Diego, Calif.) were used. The animal room was lighted artificially at a 12-hr light-dark cycle (from 7:00 A.M. to 7:00 P.M.) with water and food supply ad libitum. Animals were allocated randomly into groups. Forty-nine (49) days prior to the behavioral tests, rats received a bolus injection of STZ (75 mg/kg, i.v.). STZ was dissolved in 0.1 M sodium citrate buffer, pH 4.5 solution, at the concentration of 75 mg/ml. To ensure the development of hyperglycemia, their non-fasted levels of glucose in whole blood, obtained via tail veins, were evaluated, using a glocometer (Accucheck®, Roche Diagnostics, Palo Alto, Calif.), once a week. Animals failing to show hyperglycemic conditions (i.e., whole blood glucose >120 mg/dL) were removed from the study. Diabetic rats were treated orally with Compound 62 (5 mg/kg or 25 mg/kg, both bid) or vehicle (1 m/kg, bid), starting the date of STZ-injection. Compound 62 was dissolved in vehicle, which is composed of 96% of 0.5% CMC and 4% of 10% Tween 80. As a control, a group of naive rats received oral Compound 62 (25 mg/kg, bid) or vehicle (1 ml/kg, bid) treatment. Each group had >12 rats. Time-effect curves of the STZ diabetic rats (Compound 62 vs. Vehicle) were compared with each other, while curves of the naive rats (Compound 62 vs. Vehicle) were compared with each other. The comparisons were conducted, using two-way (group×time) repeated measures analysis of variance (ANOVA) followed by Fishers post-hoc test. A probability value of p<0.05 was considered as statistically significant.

Before the experiments, these rats were trained in the paw-withdrawal reflex test, using a Basile Analgesymeter (Ugo Basile, Biological Research Apparatus, Comerio VA, Italy), which applies a linearly increasing mechanical force to the dorsum of the rats hind paw. The training of mechanical nociceptive flexion reflex response was performed on lightly restrained rats, at 5-min intervals for 1 hr each day for a period of 5 days. On the day of the experiment, paw-withdrawal thresholds (PWT) (i.e., mechanical force that causes the animal to withdraw its paw from the stimulus) were measured at 5-min intervals for 1 hr. The mean was obtained from the average of the last 6 PWT readings. Data are presented as means±SEM (Standard Error of the Mean); One-way ANOVA was used to determine significant difference between multiple pairs of means. A probability value of p<0.05 was considered as statistically significant.

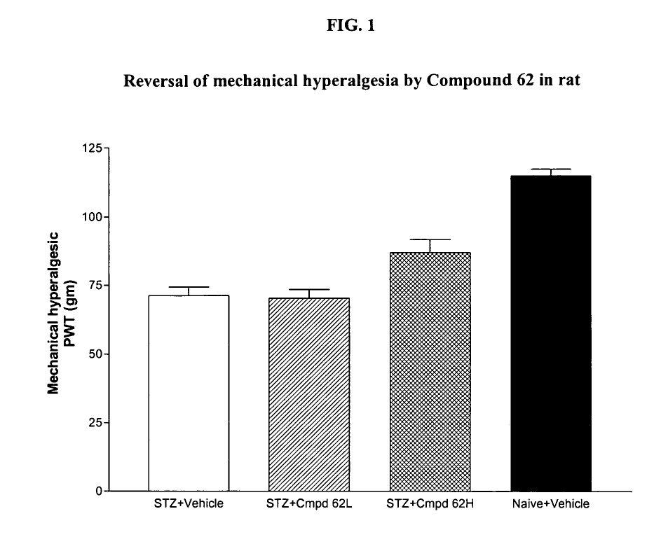

As shown in FIG. 1 and Table 7 below, high-dose Compound 62 (25 mg/kg, p.o., bid, ×49 d [STZ+Cmpd 62H, crossed-hatched bar]), but not low-dose Compound 62 (5 mg/kg, p.o., bid, ×49 d [STZ+Cmpd 62L, hatched bar]) significantly reversed mechanical hyperalgesia in STZ-diabetic rats, compared with vehicle-treated STZ-diabetic rats (STZ+Vehicle, open bar). There was no mechanical hyperalgesia in naïve rats (naïve+Vehicle, black solid bar).

TABLE 7
Reversal of Mechanical Hyperalgesia by Compound 62

| GROUP | PWT (GRAMS) |
|---|---|
| STZ + VEHICLE | 71.29 ± 3.09 |
| STZ + CMPD 62L | 70.29 ± 3.18 |
| STZ + CMPD 62H | 86.86 ± 4.79 |
| NAÏVE + VEHICLE | 114.00 ± 2.34 |

### 6.90 Example 90

### Compound 62 is Effective in Vivo against Diabetic Neuropathy (Mechanical Allodynia)

In this example, the ability of Compound 62 to produce beneficial effects in protecting against and/or reversing the pathology of neuropathy in a streptozotocin (STZ)-induced rat model of diabetes. To evaluate if chronic treatment with Compound 62 protects the diabetic animals from developing neuropathy, they were examined for mechanical allodynia responses (i.e., amplified response to non-painful tactile stimulation).

In this experiment, each rat was placed on a metal mesh floor, covered with a plastic box (10×10×18 cm), and allowed 1-2 hr to habituate. Tactile stimulation (i.e., non-painful mechanical stimulation) was induced by a set of calibrated von Frey filaments (North Coast Medical Inc., Morgan Hill, Calif.), which was applied to the plantar surface of each hind paw of the rat. The mechanical stimulation was qualified by the strength of bending force on a von Frey filament that causes the animal to withdraw its paw to avoid the pain. Each trial consisted of 4 applications of a von Frey filament given every 4 sec. Brisk foot withdrawals (i.e., PWT), at least twice out of 4 applications, in response to von Frey filament stimulation, were considered positive. Depending on the initial response, subsequent filaments were applied in the order of either descending or ascending force to determine the threshold force (Tal, M. & Bennett, G. J.: Extra-territorial pain in rats with a peripheral mononeuropathy: mechano-hyperalgesia and mechano-allodynia in the territory of an uninjured nerve. Pain, 57: 275-382, 1994; Mao, J., Price, D. D., Zhu, J., Lu, J. & Mayer, D. J.: The inhibition of nitric oxide-activated poly(ADP-ribose) synthetase attenuates transynaptic alteration of spinal cord dorsal horn neurons and neuropathic pain in the rat. Pain, 72: 355-366, 1997). Data presented as means±SEM. Results obtained from various groups of animals were compared, using a two-tailed, unpaired Students t-test. A probability value of p<0.05 was considered as statistically significant.

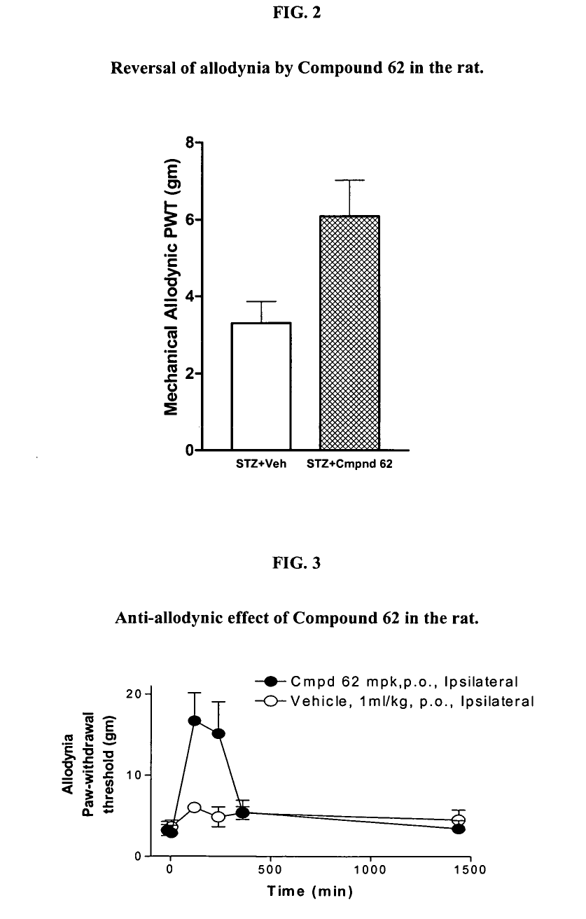

As shown in FIG. 2 and Table 8 below, Compound 62 given at 25 mg/kg, p.o., bid, ×49 d (crossed-hatched bar), but not its vehicle (open bar), significantly enhanced the PWT. An increase in PWT represents a reversal of allodynia.

TABLE 8
Enhancement of PWT by Compound 62

| GROUP | PWT (GRAMS) |
|---|---|
| STZ + VEHICLE | 3.30 ± 0.56 |
| STZ + CMPD 62 | 6.08 ± 0.94 |

### 6.91 Example 91

### Effect of Aryl Nitrone Compounds of the Invention on Mechanical Allodynia in a Rat Model of Mononeuropathic Pain

In this example, the ability of compounds of the invention to produce beneficial effects in protecting against and/or reversing the pathology of neuropathic pain, compounds were tested in a model of mononeuropathic pain.

Adult male SD rats weighing 250-300 gm (Charles River Laboratories, San Diego, Calif.) were used. The animal room was lighted artificially at a 12-hr light-dark cycle (7:00 A.M. to 7:00 P.M) with water and food supply ad libitum. Animals were allocated randomly into groups. Seven days before establishing the mononeuropathic pain disease model, rats were trained on a metal mesh floor, covered with a plastic box (10×10×18 cm) 1-2 hr per day to habituate. During the habituating phase, non-painful tactile stimulation on the plantar surface of each hind paw was induced by a set of calibrated von Frey filaments, through the mesh floor, as described in example 2.

Following the 7-day training phase, the animals were anesthetized by i.p. injection of sodium pentobarbital (65 mg/kg, Abbott Lab, Chicago, Ill.). Under aseptic procedures, the skin of the left thigh was cut open for ˜2 cm. Mid-thigh level of the common sciatic nerve was exposed after blunt separation of the muscles. Two 4-0 silk and one 4-0 chromic gut sutures (both from Ethicon, Somerville, N.J.) were loosely ligated around the nerve, with a 1-1.5 mm interval between each of them. Skin wound was then close with wound clips. The right side (i.e., the contralateral side) was not surgically injured. After recovery from surgery, rats showing post-surgery neurological deficits or poor grooming were excluded from the experiments. This surgical procedure (i.e., chronic constrictive injury, CCI, or Bennett model) to establish mononeuropathic pain disease model has been described elsewhere (Bennett, G. J. and Xie, Y. K.: A peripheral mononeuropathy in rat that produces disorders of pain sensation like those seen in man. Pain, 33: 87-107, 1988).

On days 1, 3, 4, 7, 9, 11, and 14 after surgery, animals were tested for mechanical allodynia with von Frey filaments as described previously. On or around post-surgical day 14, the ipsilateral hind paw, felt to 5 grams, an indication of allodynia began to manifest mechanical allodynia (i.e., amplified response to non-painful tactile stimulation by von Frey filaments). The contralateral hind paws remained above the 5-gram level (i.e., no allodynia).

After manifestation of this allodynia, Compound 62 (50 mg/kg, p.o.), Compound 63 (50 mg/kg, p.o.), Compound 66 (50 mg/kg, p.o.), Compound 23 (50 mg/kg, p.o.), 4-hydroxy-tempol (TEMPOL, 50 mg/kg, p.o.), or piroxicam (a COX1 inhibitor, 50 mg/kg, p.o.) were administered to randomly-assigned animals. Changes of mechanical allodynia in the ipsilateral hind paw were recorded at several time points over the course of up to 24 hr. A single dose of piroxicam induced a long-lasting anti-allodynic effect (data not shown).

As shown in FIG. 3 and Table 9 below, a single-dose of Compound 62 (filled circles), but not its vehicle (open circles), produced a rapid onset but short-lasting anti-allodynic effect by moving the PWT dramatically higher, away from the 5-gram allodynia level. Data are presented as means±SEM. The difference between the levels of allodynia over time in the ipsilateral hind paws of the CCI rats (Compound 62 vs. Vehicle) were found to be significant by two-way (group×time) repeated measures analysis of variance (ANOVA) followed by Fishers post-hoc test. A probability value of p<0.05 was considered as statically significant.

TABLE 9
Anti-allodynic effect of Compound 62 in the rat

| Post-dosing Time | | |
|---|---|---|
| (min) | Vehicle | Compound 62 |
| −15 | 3.20 ± 0.68 gm | 3.17 ± 1.10 gm |
| 5 | 3.59 ± 0.85 gm | 2.86 ± 0.94 gm |
| 120 | 6.00 ± 0.44 gm | 16.71 ± 3.44 gm |
| 240 | 4.86 ± 1.22 gm | 15.14 ± 3.90 gm |
| 360 | 5.34 ± 0.79 gm | 5.46 ± 1.46 gm |
| 1440 | 4.51 ± 1.22 gm | 3.43 ± 1.02 gm |

As shown in Table 10, a single-dose of Compound 63, but not its vehicle, produced anti-allodynic effects on ipsilateral hind paws, in a pattern similar to that of Compound 63 (i.e., rapid in onset but short in lasting). Data are presented as means±SEM. The difference between the levels of allodynia over time in the ipsilateral hind paws of the CCI rats (group compound 63 vs. group Vehicle) was found to be significant by the same analyses performed for Compound 62.

TABLE 10
Anti-allodynic effect of Compound 63 in the rat.

| Post-dosing Time | | |
|---|---|---|
| (min) | Vehicle | Compound 63 |
| −15 | 3.20 ± 0.97 gm | 3.88 ± 0.97 gm |
| 30 | 2.68 ± 0.84 gm | 18.4 ± 10.42 gm |
| 60 | 3.80 ± 0.80 gm | 18.8 ± 11.29 gm |
| 300 | 4.52 ± 1.23 gm | 5.20 ± 1.63 gm |
| 1440 | 4.28 ± 1.23 gm | 3.60 ± 1.30 gm |

As shown in Table 11, a single-dose of Compound 66 produced no significant anti-allodynic effect compared to its vehicle. Data are presented as means±SEM. Statistical analyses were performed as for compounds above.

TABLE 11
Lack of anti-allodynic effect of Compound 66 in the rat.

| Post-dosing | | |
|---|---|---|
| Time (min) | Vehicle | Compound 66 |
| −15 | 3.20 ± 0.97 gm | 1.97 ± 0.84 gm |
| 30 | 2.68 ± 0.84 gm | 5.33 ± 0.42 gm |
| 60 | 3.80 ± 0.80 gm | 4.12 ± 0.91 gm |
| 300 | 4.52 ± 1.23 gm | 5.33 ± 1.12 gm |
| 1440 | 4.28 ± 1.23 gm | 1.57 ± 0.23 gm |

As shown in Table 12, a single-dose of Compound 23, but not its vehicle, produced statistically significant anti-allodynic effects on ipsilateral hind paws. Data are presented as means±SEM. Statistical analyses were performed as for compounds above.

TABLE 12
Anti-allodynic effect of Compound 23 in the rat.

| Post-dosing Time | | |
|---|---|---|
| (min) | Vehicle | Compound 23 |
| −15 | 3.20 ± 0.97 gm | 2.43 ± 0.54 gm |
| 30 | 2.68 ± 0.84 gm | 7.67 ± 0.80 gm |
| 60 | 3.80 ± 0.80 gm | 8.17 ± 1.52 gm |
| 300 | 4.52 ± 1.23 gm | 4.43 ± 1.35 gm |
| 1440 | 4.28 ± 1.23 gm | 4.67 ± 0.84 gm |

As shown in Table 13, a single-dose of compound TEMPOL produced no significant anti-allodynic effect compared to its vehicle. Data are presented as means±SEM. Statistically analyses were performed as for compounds above.

TABLE 13
Lack of anti-allodynic effect of TEMPOL in the rat.

| Post-dosing Time | | |
|---|---|---|
| (min) | Vehicle | TEMPOL |
| −15 | 3.20 ± 0.97 gm | 4.00 ± 0.89 gm |
| 30 | 2.68 ± 0.84 gm | 4.80 ± 0.49 gm |
| 60 | 3.80 ± 0.80 gm | 4.80 ± 0.49 gm |
| 300 | 4.52 ± 1.23 gm | 5.48 ± 1.09 gm |
| 1440 | 4.28 ± 1.23 gm | 4.80 ± 1.02 gm |

### 6.92 Example 92

### Aryl Nitrone Compound of the Invention Decrease Thermal Hyperalgesia in Acute Inflammation produced by Carrageenan in Rats

In this example, the ability of compounds of the invention to decrease thermal hyperalgesia under acute inflammatory conditions is demonstrated. A carrageenan-sensitized inflamamatory model of rat was used and compounds were tested for their effects on response to thermal pain using Hargreaves test.

In this experiment, animals were habituated to the test environment for 2 days. Each rat was individually placed on a transparent perplex glass floor, covered with a plastic box (10×10×18 cm), and allowed 0.5-1 hr to habituate. After the acclimation period, basal thermal withdrawal latency (PWL, time interval between heat stimulation and paw withdrawal) was measured by exposing the plantar surface of a rats hind paw to a beam of radiant heat generated from a focused projection bulb through a transparent perplex glass surface (Hargreaves test; Hargreaves, K. R., Dubner, R., Brown, F., Flores, C. & Joris, J.: A new and sensitive method for measuring thermal nociception in cutaneous hyperalgesia. Pain, 32: 77-88, 1988). The PWL was averaged from at least two trials separated by a 2 min interval. A timer was used to measure the withdrawal latency and a cut of time of 20-sec was used to prevent tissue damage. Before the test on STZ-diabetic rats, the intensity of the radiating heat was adjusted to the level that caused naive animal to withdraw its paw at around 10 seconds.

After a stable basal PWL was obtained, the animals were briefly anesthetized with isoflurane (2-5% to effect). One side of their hind paws received an intraplantar injection of lambda carrageenan (2 mg in 100 microliter sterile saline, Sigma, St. Louis, Mo.). The contralateral side received no injection and served as an intra-subject control. The animals were then returned to their home cages and transferred to the individual testing chambers for heat thermal hyperalgesic testing, 2.5 hr after carrageenan injection. 3-hr post-carrageenan injection, PWLs of both hind paws (i.e., ipsilateral and contralateral) were measured at several time points post-carrageenan injection (15-min to 24-hr). Without any anti-hyperalgesic intervention, PWL of the ipsilateral hind paws was significantly lower than the non-injected contralateral hind paws until the spontaneous recovery at 24-hr time point (data not shown).

For compound testing, rats were randomly enrolled into groups that, immediately after the 3-hr post-carrageenan PWL was obtained, received oral dosing of Compound 62 (50 mg/kg), Compound 63 (50 mg/kg), vehicle (1 ml/kg), or indomethacin (30 mg/kg). Compound 62 and Compound 63 were prepared as a suspension in vehicle (96% of 0.5% CMC and 4% of 10% Tween 80) while indomethacin was prepared as a 30 mg/ml in normal saline. Orally-administered indomethacin significantly reversed carrageenan-sensitized heat hyperalgesia (data not shown).

As shown in Table 14, compound 62 produced a statistically significant effect compared to vehicle-treated carrageenan-paw. In Table 14, data are presented as means±SEM. The method of statistical comparison used in this study was a two-way repeated measures ANOVA followed by Fishers post-hoc test. A probability value of p<0.05 was considered as statistically significant.

TABLE 14
Reversal of carrageenan-sensitized heat hyperalgesia
by Compound 62 in the rat.

| | Post-dosing time (min) | | | | |
|---|---|---|---|---|---|
| Group | 0 | 15 | 120 | 240 | 1400 |
| PWL for | 3.29 ± 0.70 | 3.15 ± 0.77 | 3.08 ± 0.81 | 3.61 ± 0.90 | 5.60 ± 0.70 |
| Vehicle (sec) | | | | | |
| PWL for Cmpd | 4.65 ± 0.63 | 7.33 ± 1.43 | 7.55 ± 1.08 | 5.38 ± 1.11 | 5.33 ± 1.09 |
| 62 (sec) | | | | | |

As shown in Table 15, compound 63 also produced a statistically significant effect compared to vehicle-treated carrageenan-paw. In Table 15, data are presented as means±SEM. Statistical methods used were same as above.

TABLE 15
Reversal of carrageenan-sensitized heat hyperalgesia
by Compound 63 in the rat.

| | Post-dosing time (min) | | | | | |
|---|---|---|---|---|---|---|
| Group | 0 | 15 | 60 | 180 | 240 | 1400 |
| PWL for | 3.29 ± 0.70 | 3.15 ± 0.77 | not | 3.43 ± 0.51 | 3.61 ± 0.90 | 5.60 ± 0.70 |
| Vehicle | | | collected | | | |
| (sec) | | | | | | |
| PWL for | 4.98 ± 0.85 | 4.93 ± 1.22 | 4.15 ± 1.04 | 3.65 ± 0.64 | not | 5.70 ± 0.71 |
| Cmpd 63 | | | | | collected | |
| (sec) | | | | | | |

### 6.93 Example 93

### Compound 62 on Alleviates Renal Dysfunction in a Kidney Ischemia-Reperfusion Injury Model

In this example, the ability of Compound 62 to protect or reverse the damage caused by ischemia-reperfusion (I/R) injury of the kidney is demonstrated. A one-kidney one-clip (i.e., 1K1C) I/R model was used.

Rats were individually housed in a modified cage that was equipped with a raised mesh bottom to separate the fecal product from urine. Before the test, all animals were withheld from food and water overnight. In the morning of the test, normal saline (i.e., 0.9% sodium chloride) was given via oral gavage at 50 mg/kg (Lipschitz, W. L. Hadidian, Z. & Kerpcar, A.: Bioassay of diuretics., J. Pharmacol. Exp. Ther., 79: 97-110, 1943). Samples of blood and urine were collected (standard procedures) at time points (1 and/or 5 hrs after fluid intake) from animals, centrifuged, and kept at 4 degrees until analysis for factors that reflect renal functions. Sodium and creatinine levels were determined by Quality Clinical Labs, Inc. (Mountain View, Calif.). Sodium concentrations were determined by ion selective electrode (standard procedures). Creatinine levels were determined by the alkaline picrated (Jaffe) reaction as described (Liobat-Estelles, M., Sevillano-Cabeja, A. & Campines-Falco, P.: Kinetic chemometric studies of the determination of creatinine using the Jaffe reaction. Part I: kinetics of the reaction; analytical conclusion. Analyst, 11: 597-602, 1989). Fractional excretion of sodium (FE_{Na+}), a parameter for ion-handling by the kidney, was calculated, using the following equation:=U_{Na}×P_{cr}/P_{Na}×U_{cr}, where U_{Na} is the concentration of sodium in urine; P_{cr} is the plasma concentration of creatinine; P_{Na} is the urine concentration of sodium; and U_{cr} is the concentration of creatinine in the urine.

After the data for the calculation of basal levels of FE_{Na+} was obtained (Table 16), rats were anesthetized with pentobarbital (65 mg/kg, i.p.). The abdominal region was shaved with a safety razor and sterilized with povidone iodine solution. A midline incision was made and the right kidney was exposed. The right renal pedicle and right urether were both ligated twice with 4-0 sutures and cut between the ligations. The right kidney was then removed. 7 days later, these rats, after another overnight food-water deprivation, were randomly assigned into 3 groups and orally given either Compound 62 (50 mg/kg, at the volume of 1 ml/kg, p.o.) or its vehicle (i.e., 96% of 0.5% CMC and 4 of 20% Tween 80, at the volume of 1 ml/kg, p.o.) 1 hr before kidney ischemia was produced. Positive controls were performed using quercetin (30 mg/kg, i.p.) given 2 hrs before ischemia (Kahraman, A., Erkasap, N., Serteser, M. & Koken, T.: Protective effect of quercetin on renal ischemia/reperfusion injury in rats. J. Nephrol., 16: 219-224, 2003). Animals were then anesthetized and had their left kidneys exposed after opening of their abdominal cavities. A non-traumatic vascular clamp was applied to the left renal pedicle (the ischemia phase), which was released 45 min later (the reperfusion phase). Lipschitz test was then conducted to measure ion-handling capabilities. Normal saline was orally administered at 50 mg/kg as soon as the vascular clamp was released. 60- and 300-min after reperfusion, plasma and urine samples were obtained.

Compared with the vehicle treatment group, acutely-dosed Compound 62 significantly enhanced FE_{Na+} 5-hr after reperfusion and the level of in this group (i.e., Compound 62-treated 1K1C rats) was significantly higher than the levels obtained from the same animals before 1K1C modeling (Table 17). The effects of Compound 62 were similar to that of quercetin, although smaller than the later. Data are presented as means±SEM. The data was processed by a two-way repeated measures ANOVA followed by Fishers post-hoc test. A probability value of p<0.05 was considered as statistically significant.

TABLE 16
Lack of effect of Compound 62 on pre-surgery
levels of FE_{Na+} in rats.

| Group | FE_{Na+} (%) |
|---|---|
| Vehicle | 1.49 ± 0.10 |
| Cmpd 62 | 1.71 ± 0.36 |
| Quercetin | 1.60 ± 0.15 |

TABLE 17
Effect of Compound 62 on levels Of FE_{Na+}
in post-kidney ischemic rats.

| | 1-hr post-ischemic | 5-hr post-ischemic |
|---|---|---|
| Group | FE_{Na+} (%) | FE_{Na+} (%) |
| Vehicle | 0.30 ± 0.10 | 1.20 ± 0.22 |
| Cmpd 62 | 0.52 ± 0.29 | 2.52 ± 0.5 |
| Quercetin | 0.54 ± 0.02* | 5.23 ± 4.43 |

### 6.94 Example 94

### Compound Effects on Alleviation of Damage from Stroke in the Rat

In this example, the ability of compounds of the invention to reduce the infarct volume in an in vivo stroke model is demonstrated. A rat model of focal ischemia, transient middle cerebral artery occlusion (tMCAO), was used. MCA occlusion was induced by the intraluminal filament technique described by Bederson et al (Rat Middle Cerebral Artery occlusion: evaluation of the model and development of a neurologic examination. Stroke Vol 17 (3) (1986) pp. 472-476)]. Male Sprague-Dawley rats (270-300g) were anesthetized with 2.5% isofluorane. During the procedure, core body temperature was maintained around 37° C. with a heating pad attached to a rectal thermometer and a temperature controller. The animal's neck was shaved and prepped with betadine and alcohol. An incision was made just below the mandibles, extending approximately 1-2 cm caudally. Blunt dissection was performed to expose the trachea and retract the muscles to locate the right carotid artery. Similarly, the bifurcation of the external common carotid artery (ECA) and the internal common carotid artery (ICA) were exposed. A silk suture was placed rostrally around the ECA, followed by a second suture next to the first. Both sutures were then tied closed and the artery severed between the sutures. Sham operated animals received no further surgery. Their incision was sutured and they were allowed to recover as described below before being returned to their home cage.

On animals undergoing 120 minute MCAO, the suture on the proximal portion of the ECA was pulled caudally so that the ECA and the ICA formed a straight line at the bifurcation. Another temporary tie was placed on the ECA just above the bifurcation to hold the monofilament in place. Blood flow through the common carotid artery (CCA) and ICA was temporarily stopped using a curved vascular clamp. A small hole was made using iridectomy scissors above the temporary tie and just below the permanent tie on the stump of the external carotid artery. A 3-0 monofilament nylon suture, pretreated with a cauteriser to flare the tip was placed into the ECA stump past the temporary tie, which was then tightened slightly to prevent blood loss. The vascular clamp was then released and the suture advanced into the lumen of the ICA. The temporary clip on the CCA/ECA/ICA bifurcation was removed and the monofilament advanced up the ICA until proper resistance was encountered. At this point, MCAO was assumed and the filament left in this position for the duration of the ischemic insult (120 mins). The suture was held in place by tightening the suture on the ECA and cutting off the loose ends. The entire region was irrigated with saline, and the incision closed using surgical staples.

At the end of the occlusion period, the animal was put under isofluorane anesthesia, the surgical staples removed and the monofilament taken out of the carotid artery. The temporary suture on the ECA was permanently ligated to prevent blood loss. Reflow was established back into the ICA, the area was irrigated with saline and the animal's incision closed with Ethilon No.5 or equivalent. Two days post MCAO, the rats were sacrificed and the extent of brain damage assessed using tetrazolium (TTC) staining on 2 mm thick sections prepared using standard methods followed by computer image analysis to quantitate infarct volumes (i.e. the regions of dead tissue). A Wilcoxan Rank Sum test (as pre-specified to follow a one way analysis of variance) was used to compare specific, compound-treatment groups with the Vehicle treated control.

Three studies were performed. All studies used the aforementioned 2 hr tMCAO model and they each followed the same dosing regimen whereby the drug was dosed BID (bi-daily), every 12 hrs, commencing 48 hrs prior to occlusion and through to sacrifice at 48 hrs post-occlusion. The animals received a total of 8 doses of drug and MCAO was performed 1 hr after the fifth dose.

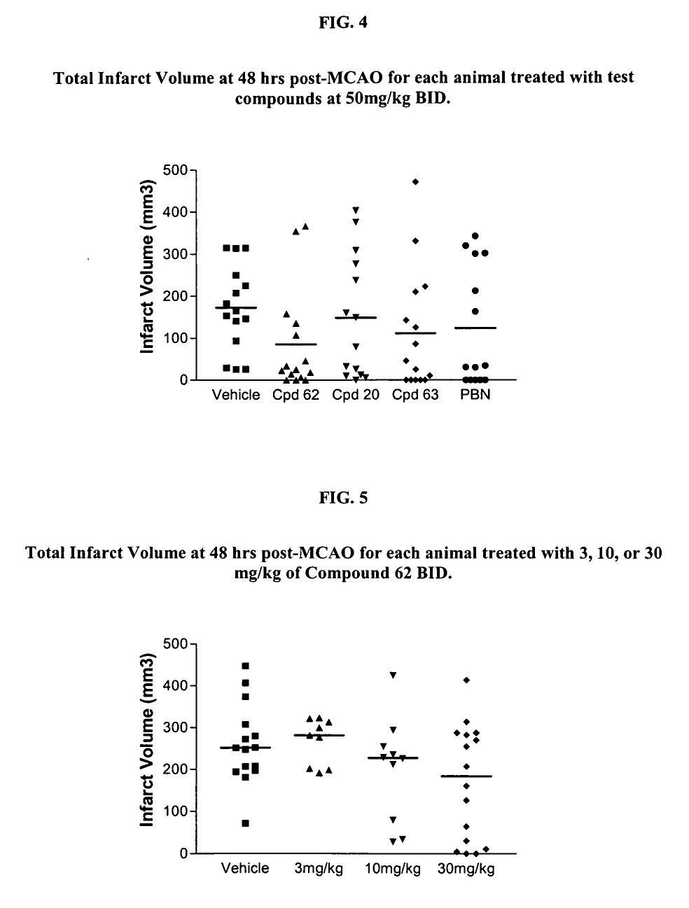

The first study compared the effects of Compound 62 (50 mg/kg), Compound 63 (50 mg/kg), and Compound 20 (50 mg/kg) to vehicle treated controls. Phenyl-N-butyl-nitrone (PBN, 100 mg/kg) administered intraperitoneally (i.p.) once 15 minutes prior to occlusion and then BID (every 12 hrs) until sacrifice at 48 hrs, was used as a positive control. There were approximately 15 rats in each experimental group. In FIG. 4, data are graphed with bars representing median values for each group. Results from statistical analyses showed statistically significant effects for some compounds: for the Compound 62 treated group compared to vehicle, (p=0.01); for the Compound 63 treated group compared to vehicle, p=0.05; for the Compound 20 treated group compared to vehicle, p=0.54; and for the PBN treated group compared to vehicle, p=0.28.

The second study looked for a dose response relationship of Compound 62 treatment on infarct volume. There were three doses used in this experiment: 3, 10, and 30 mg/kg administered via oral gavage BID starting 48 hrs prior to MCAO and continuing until the end of the study, 48hrs post-MCAO. In FIG. 5, data are graphed with bars representing median values for each group. Results from statistical analyses showed statistically significant effects for Compound 62: p=0.03 for the 30 mg/kg dose group compared to vehicle.

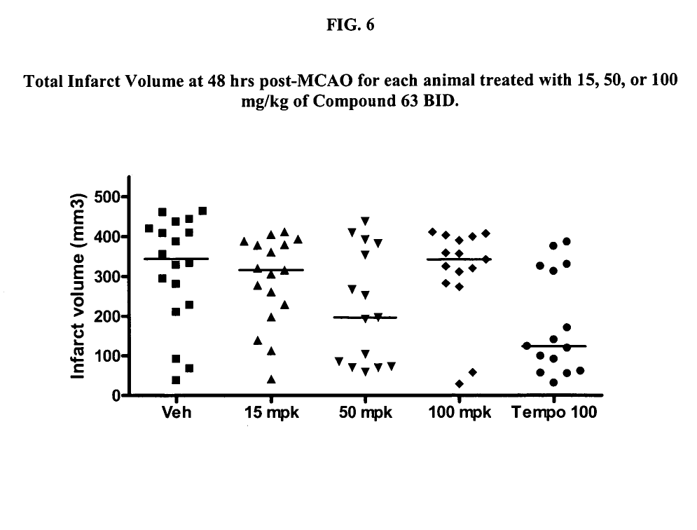

The third study looked for a dose response relationship of Compound 63 treatment on infarct volume. There were three doses of Compound 63 used in this experiment: 15, 50, and 100 mg/kg administered via oral gavage BID starting 48 hrs prior to MCAO and continuing until the end of the study: 48hrs post-MCAO. 4-hydroxy-TEMPO (100 mg/kg) was used as a positive control and was administered using the same dosing regimen. In FIG. 6, data are graphed with bars representing median values for each group. Results from statistical analyses showed possible effects for Compound 63 (50 mg/kg), p=0.07 for the 50 mg/kg dose group compared to vehicle.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. All such changes and modifications included herein.

1. A compound of formula (1), 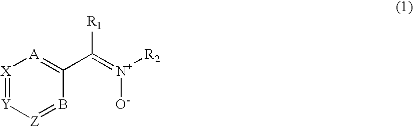
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
at least one of A and B is C—R³, and the other is selected from C—R³ and N;
at least one R³ is SO₂R⁵, CO₂R⁵, CONR⁵R⁶ or tetrazole, and any other R³ is independently selected from R⁴, H, lower alkyl, alkenyl, alkyl, halogen, aryl, SO₂R⁵, SO₂NR⁵R⁶, CO₂H, CONR⁵R⁶ and tetrazole;
X, Y and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S,
wherein the compound is not α-2-carboxy-phenyl-N-t-butyl-nitrone, α-2-carboxy-phenyl-N-phenyl-nitrone, α-2-carboxy-phenyl-N-3,4-dimethyl-phenyl-nitrone, or α-2-carboxy-3,4-dimethoxy-phenyl-N-methyl-nitrone.

2. A compound according to formula (2), (3) or (4): 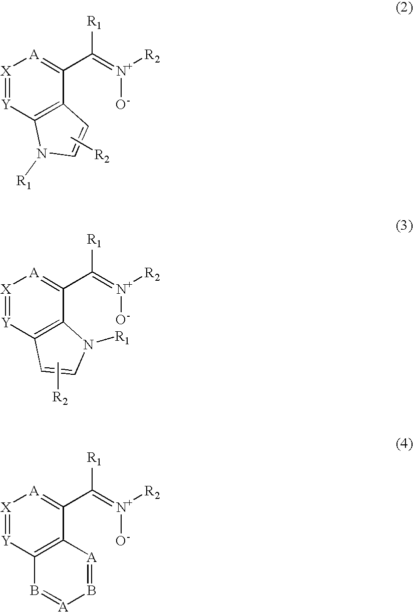
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
A and B are independently selected from C—R³ and N;
each R³ is independently selected from R⁴, H, lower alkyl, alkenyl, alkyl, halogen, aryl, SO₂NR⁵R⁶, SO₂R⁵, CO₂H, CONR⁵R⁶ and tetrazole;
X, Y and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S;
wherein the A adjacent to the nitrone group is SO₂R⁵, CO₂R⁵, CONR⁵R⁶ or tetrazole.

3. A compound according to formula (5) or (6): 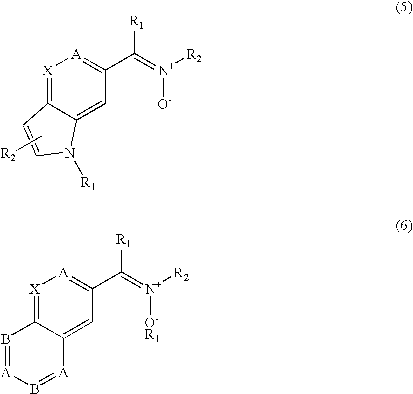
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
A and B are independently selected from C—R³ and N;
each R³is independently selected from R⁴, H, lower alkyl, alkenyl, alkyl, halogen, aryl, SO₂NR⁵R⁶, SO₂R⁵, CO₂H, CONR⁵R⁶ and tetrazole;
X, Y and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S;
wherein the A adjacent to the nitrone group is SO₂R⁵, CO₂R⁵, CONR⁵R⁶ or tetrazole.

4. (Canceled)

5. (Canceled)

6. (Canceled)

7. (Canceled)

8. (Canceled)

9. (Canceled)

10. (Canceled)

11. (Canceled)

12. (Canceled)

13. (Canceled)

14. (Canceled)

15. (Canceled)

16. (Canceled)

17. A pharmaceutical composition comprising a compound as claimed in any of claims 1-3, and a pharmaceutically acceptable carrier, excipient or diluent.

18. A unit dosage form of the composition of claim 17 comprising about 10, 25, 50, 100, 500, 1000, 2000 or 2500 mg of the compound.

19. A method of treating or preventing an ischemic or ischemia/reperfusion-related condition comprising the step of administering an effective amount of the compound as claimed in any of claims 1-3 to a subject in need of said treating or preventing.

20. A method of treating or preventing a chemokine mediated condition comprising the step of administering an effective amount of the compound as claimed in any of claims 1-3 to a subject in need of said treating or preventing.

21. (Canceled)

22. (Canceled)

23. (Canceled)

24. A kit for treating or preventing an ischemic or ischemia/reperfusion-related or a chemokine mediated condition in a subject in need thereof comprising an effective amount of a pharmaceutical composition, said composition comprising compound as claimed in any of claims 1-3, and a label or labeling with instructions for using the composition to treat or prevent the condition.

25. A method of making an aryl nitrone according to claim 1 comprising the step of reacting an aldehyde according to formula (7) 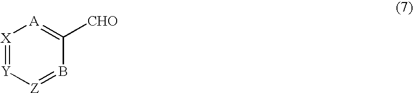
with a hydroxylamine according to formula (8) 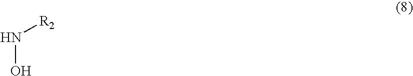
to yield the aryl nitrone according to formula (1).

26. A compound of formula (11), 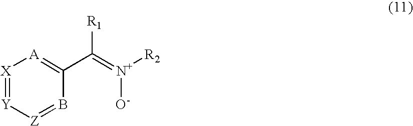
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
at least one of A and B is C—R³, and the other is selected from C—R³ and N;
at least one R³ is SO₂NR⁵R⁶, and the other R³ is independently selected from R⁴, H, lower alkyl, alkenyl, alkyl, halogen, aryl, SO₂NR⁵R⁶, SO₂R⁵, CO₂H, CONR⁵R⁶ and tetrazole;
X, Y and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S.

27. A compound according to formula (12), (13) or (14): 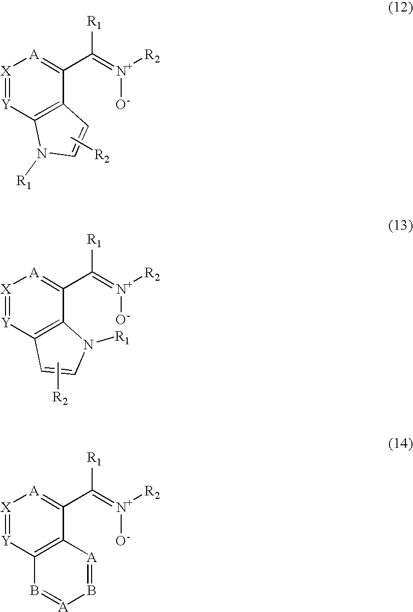
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
A and B are independently selected from C—R³ and N;
each R³ is independently selected from R⁴, H, lower alkyl, alkenyl, alkyl, halogen, aryl, SO₂NR⁵R⁶, SO₂R⁵, CO₂H, CONR⁵R⁶ and tetrazole;
X, Y and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S;
wherein the A adjacent to the nitrone group is C—SO₂NR⁵R⁶.

28. A compound according to formula (15) or (16): 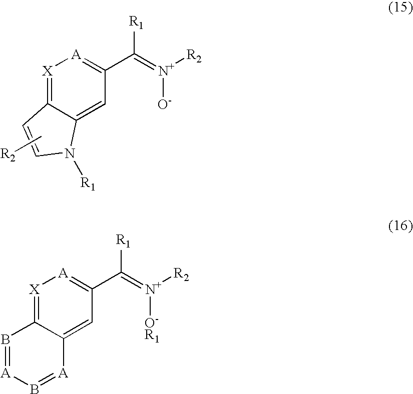
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
A and B are independently selected from C—R³ and N;
each R³ is independently selected from R⁴, H, lower alkyl, alkenyl, alkyl, halogen, aryl, SO₂NR⁵,R⁶, SO₂R⁵, CO₂H, CONR⁵R⁶ and tetrazole;
X, Y and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S;
wherein the A adjacent to the nitrone group is C—SO₂NR⁵R⁶.

29. (Canceled)

30. (Canceled)

31. (Canceled)

32. (Canceled)

33. (Canceled)

34. (Canceled)

35. (Canceled)

36. (Canceled)

37. A pharmaceutical composition comprising a compound as claimed in any of claims 26-28, and a pharmaceutically acceptable carrier, excipient or diluent.

38. A unit dosage form of the composition of claim 37 comprising about 10, 25, 50, 100, 500, 1000, 2000 or 2500 mg of the compound.

39. A method of treating or preventing an ischemic or ischemia/reperfusion-related condition comprising the step of administering an effective amount of the compound as claimed in any of claims 26-28 to a subject in need of said treating or preventing.

40. A method of treating or preventing a chemokine mediated condition comprising the step of administering an effective amount of the compound as claimed in any of claims 26-28 to a subject in need of said treating or preventing.

41. (Canceled)

42. (Canceled)

43. (Canceled)

44. A kit for treating or preventing an ischemic or ischemia/reperfusion-related or a chemokine mediated condition in a subject in need thereof comprising an effective amount of a pharmaceutical composition, said composition comprising compound as claimed in any of claims 26-28, and a label or labeling with instructions for using the composition to treat or prevent the condition.

45. A method of making an aryl nitrone according to claim 26 comprising the step of reacting an aldehyde according to formula (17) 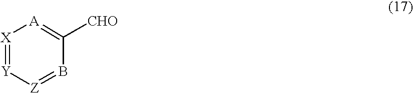
with a hydroxylamine according to formula (18) 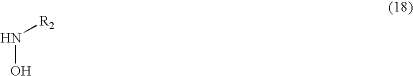
to yield the aryl nitrone according to formula (11).

46. A compound of formula (21), 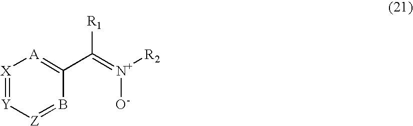
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
Y is C—R⁹, and R⁹ is selected from SO₂NR⁵R⁶, SO₂R⁵, CO₂R⁵, CONR⁵R⁶ and tetrazole;
A, B, X and Z are each independently selected from C—R⁴and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S,
wherein the compound is not any of compounds 401-426.

47. A compound according to formula (22), (23) or (24): 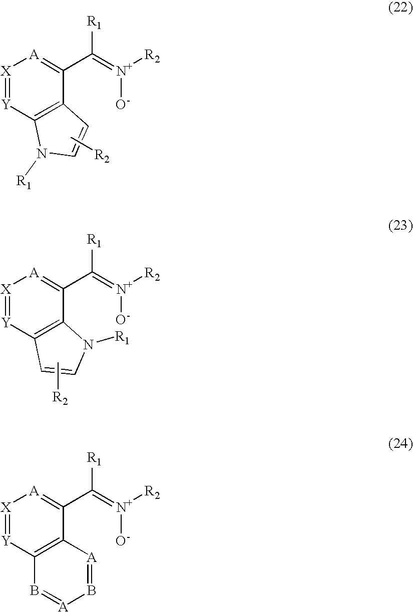
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
Y is C—R⁹, and R⁹ is selected from SO₂NR⁵R⁶, SO₂R⁵, CO₂R⁵, CONR⁵R⁶ and tetrazole;
A, B, X and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S.

48. A compound according to formula (25) or (26): 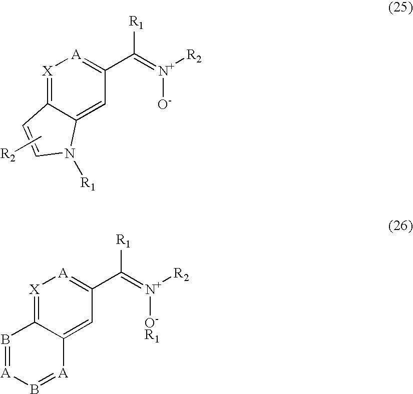
or a pharmaceutically acceptable salt or solvate thereof, wherein:
R¹ is selected from H, lower alkyl and alkyl;
R² is selected from lower alkyl, alkyl, aryl, arylalkyl, cycloalkyl, heteroaryl, heteroarylalkyl and cycloheteroalkyl;
Y is C—R⁹, and R⁹ is selected from SO₂NR⁵R⁶, SO₂R⁵, CO₂R⁵, CONR⁵R⁶and tetrazole;
A, B, X and Z are each independently selected from C—R⁴ and N;
each R⁴ is independently selected from hydrogen, alkyl, substituted alkyl, acyl, substituted acyl, acylamino, substituted acylamino, alkylamino, substituted alkylamino, alkylthio, substituted alkylthio, alkoxy, substituted alkoxy, alkoxycarbonyl, substituted alkoxycarbonyl, alkylarylamino, substituted alkylarylamino, arylalkyloxy, substituted arylalkyloxy, amino, aryl, substituted aryl, arylalkyl, substituted arylalkyl, sulfoxide, substituted sulfoxide, sulfone, substituted sulfone, sulfanyl, substituted sulfanyl, aminosulfonyl, substituted aminosulfonyl, arylsulfonyl, substituted arylsulfonyl, sulfuric acid, sulfuric acid ester, dihydroxyphosphoryl, substituted dihydroxyphosphoryl, aminohydroxyphosphoryl, substituted aminohydroxyphosphoryl, azido, carboxy, carbamoyl, substituted carbamoyl, carboxyl, cyano, cycloalkyl, substituted cycloalkyl, cycloheteroalkyl, substituted cycloheteroalkyl, dialkylamino, substituted dialkylamino, halo, heteroaryloxy, substituted heteroaryloxy, heteroaryl, substituted heteroaryl, heteroalkyl, substituted heteroalkyl, hydroxyl, nitro or thio; and
R⁵ and R⁶ are each independently selected from H, lower alkyl, alkyl, aryl and heteroaryl, or join together to form a saturated or unsaturated cycloheteroalkyl ring containing 4 to 8 atoms, having zero, one or more heteroatoms selected from NR¹, O and S.

49. (Canceled)

50. (Canceled)

51. (Canceled)

52. (Canceled)

53. (Canceled)

54. (Canceled)

55. (Canceled)

56. (Canceled)

57. (Canceled)

58. (Canceled)

59. (Canceled)

60. (Canceled)

61. (Canceled)

62. A pharmaceutical composition comprising a compound as claimed in any of claims 46-48, and a pharmaceutically acceptable carrier, excipient or diluent.

63. A unit dosage form of the composition of claim 62 comprising about 10, 25, 50, 100, 500, 1000, 2000 or 2500 mg of the compound.

64. A method of treating or preventing an ischemic or ischemia/reperfusion-related condition comprising the step of administering an effective amount of the compound as claimed in any of claims 46-48 to a subject in need of said treating or preventing.

65. A method of treating or preventing a chemokine mediated condition comprising the step of administering an effective amount of the compound as claimed in any of claims 46-48 to a subject in need of said treating or preventing.

66. (Canceled)

67. (Canceled)

68. (Canceled)

69. A kit for treating or preventing an ischemic or ischemia/reperfusion-related or a chemokine mediated condition in a subject in need thereof comprising an effective amount of a pharmaceutical composition, said composition comprising compound as claimed in any of claims 46-48, and a label or labeling with instructions for using the composition to treat or prevent the condition.

70. A method of making an aryl nitrone according to claim 46 comprising the step of reacting an aldehyde according to formula (27) 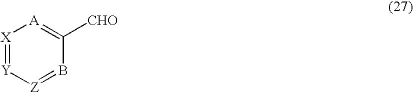
with a hydroxylamine according to formula (28) 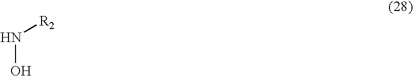
to yield the aryl nitrone according to formula (21).

71. A compound selected from the group consisting of compounds 1-81, or a pharmaceutically acceptable salt or solvate thereof.

72. A pharmaceutical composition comprising a compound as claimed in claim 71, and a pharmaceutically acceptable carrier, excipient or diluent.

73. A unit dosage form of the composition of claim 72 comprising about 10, 25, 50, 100, 500, 1000, 2000 or 2500 mg of the compound.

74. A method of treating or preventing an ischemic or ischemia/reperfusion-related condition comprising the step of administering an effective amount of the compound as claimed in claim 71 to a subject in need of said treating or preventing.

75. A method of treating or preventing a chemokine mediated condition comprising the step of administering an effective amount of the compound as claimed in claim 71 to a subject in need of said treating or preventing.

76. (Canceled)

77. (Canceled)

78. (Canceled)

79. A kit for treating or preventing an ischemic or ischemia/reperfusion-related or a chemokine mediated condition in a subject in need thereof comprising an effective amount of a pharmaceutical composition, said composition comprising the compound as claimed in claim 71, and a label or labeling with instructions for using the composition to treat or prevent the condition.
